(19) United States
(12) Patent Application Publication
(10) Pub. No.: US 2006/0252748 A1
(43) Pub. Date: Nov. 9, 2006

(54) Use of CDK II inhibitors for birth control

(76) Inventors: Bernhard Lindenthal, Berlin (DE); Gerhard Siemeister, Berlin (DE); Andrea Wagenfeld, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/355,201

(22) Filed: Feb. 16, 2006

Related U.S. Application Data
(60) Provisional application No. 60/664,552, filed on Mar. 24, 2005.

(30) Foreign Application Priority Data
Feb. 17, 2005 (DE) 102005008310.2

(51) Int. Cl. A61K 31/513; A61K 31/506

(52) U.S. Cl. 514/218; 514/235.2; 514/252.11; 514/252.15; 514/252.18

(57) ABSTRACT

The subject of this invention is a method for birth control comprising the inhibition of CDK II as well as the use of CDK II inhibitors of general formulas I and II for the development of a pharmaceutical agent for contraception.

This invention relates to a method for birth control, comprising the use of CDK II inhibitors.

For birth control, the broad spectrum of the “standard pill” as a contraceptive agent is available to women, but only the condom or sterilization is approved at this time for men.

Taking the pill regularly leads to ovulation inhibition in women, since it results in suppressing the endogenic steroid hormone production in the ovary by the daily intake of hormones. A drawback of this regular intake consists in that a natural cycle thus is no longer present in women. Moreover, in connection with female patients who are potentially at risk in taking a hormonal preparation, side effects, such as, for example, tender breasts and weight gain, can occur.

There is therefore a need for the development of new reliable agents both for female and for male birth control. In this connection, the infertility that is produced by the substance administration is completely reversible and effective. The infertility should set in relatively quickly and be as long-lasting as possible. Such a contraceptive method should have the fewest possible side effects. To overcome the drawbacks of the known hormonal preparations, these should be non-hormonal preparations in this connection.

The influencing of the maturation of female egg cells (oogenesis) and that of sperm (spermatogenesis) could represent a possible starting point for a common concept for birth control of women and men. Oogenesis and spermatogenesis are processes in whose regulation the enzyme cyclin-dependent kinase II (cyclin-dependent kinase=CDK II) is involved.

CDK 2 is an enzyme kinase whose function in the mitotic and meiotic division of cells is described and that is activated in specific phases of the cell cycle.

For some time, it could be shown that CDK II knock-out mice, which, were unremarkable over a period of up to 2 years and also showed no anatomical characteristics, were completely infertile. This infertility was probably caused by an obvious atrophy of the gonads. Male CDK II^{-/-} mice did not show any round spermatides in the seminiferous tubules. Postmeiotic cells (spermatocytes, spermatides and spermatozoa) were no longer present. Adult female animals show an atrophic ovary without egg cells and follicles (Ortega et al., Nature Genetics, 2003); Berthet et al.; Current Biology, 2003).

The prior art points out that in the CDK II^{-/-} animals, a general atrophy and irreversible damage of the gonads result by eliminating the CDK II gene. This strong tissue-destroying effect does not appear to be a very promising basis for creating a preparation for birth control with the inhibition of CDK II.

Consequently, it is not known to date whether an inhibitor of the CDK II could have a specific action on the maturation of germ cells. The achievement of such an effect would presuppose that a potential CDK II inhibitor is able to pass through the cell membrane to prevent the maturation of gametes in the cell by specific CDK II inhibition. At the same time, it would be expected that structurally similar kinases are also inhibited and thus strong side effects occur.

A more selective inhibitor of the CDK II, however, could be used as a pharmaceutical agent for birth control for women and men.

Although the use of CDK II inhibitors for contraception relative to male fertility in a number of applications is disclosed generically in a variety of patent applications (US20030078252, US20020119963, WO02010141, WO02018346, US20030199525), to date any reference to biological action in vitro and in vivo is lacking.

In a surprising way, it was now found that compounds of general formula I 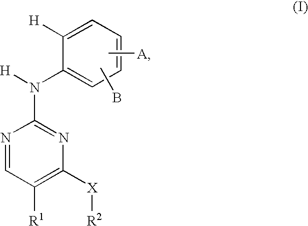
in which
R¹ stands for hydrogen, halogen, C₁-C₆-alkyl, nitro or for the group —COR⁵, —OCF₃, —(CH₂)ₙR⁵, —S—CF₃ or —SO₂CF₃,
R² stands for C₁-C₁₀-alkyl, C₂-C₁₀-alkenyl, C₂-C₁₀-alkinyl or C₃-C₁₀-cycloalkyl or for a C₁-C₁₀-alkyl, C₂-C₁₀-alkenyl, C₂-C₁₀-alkinyl or C₃-C₁₀-cycloalkyl that is substituted in one or more places, in the same way or differently, with hydroxy, halogen, C₁-C₆-alkoxy, C₁-C₆-alkylthio, amino, cyano, C₁-C₆-alkyl, —NH—(CH₂)ₙ—C₃-C₁₀-cycloalkyl, C₃-C₁₀-cycloalkyl, C₁-C₆-hydroxyalkyl, C₂-C₆-alkenyl, C₂-C₆-alkinyl, C₁-C₆-alkoxy-C₁-C₆-alkyl, C₁-C₆-alkoxy-C₁-C₆-alkoxy-C₁-C₆-alkyl, —NHC₁-C₆-alkyl, —N(C₁-C₆-alkyl)₂, —SO(C₁-C₆-alkyl), —SO₂(C₁-C₆-alkyl), C₁-C₆-alkanoyl, —CONR³R⁴, —COR⁵, C₁-C₆-alkylOAc, carboxy, aryl, heteroaryl, —(CH₂)ₙ-aryl, —(CH₂)ₙ-heteroaryl, phenyl-(CH₂)ₙ—R⁵, —(CH₂)ₙPO₃(R⁵)₂ or with the group —R⁶ or —NR³R⁴, and the phenyl, C₃-C₁₀-cycloalkyl, aryl, heteroaryl, —(CH₂)ₙ-aryl and —(CH₂)ₙ-heteroaryl itself optionally can be substituted in one or more places, in the same way or differently, with halogen, hydroxy, C₁-C₆-alkyl, C₁-C₆-alkoxy, heteroaryl, benzoxy or with the group —CF₃ or —OCF₃, and the ring of C₃-C₁₀-cycloalkyl and C₁-C₁₀-alkyl optionally can be interrupted by one or more nitrogen, oxygen and/or sulfur atoms, and/or can be interrupted by one or more ═C═O groups in the ring and/or optionally one or more possible double bonds can be contained in the ring, or
R² stands for the group 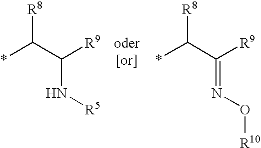
X stands for oxygen or for the group —NH—, —N(C₁-C₃-alkyl) or for —OC₃—C₁₀-cycloalkyl, which can be substituted in one or more places, in the same way or differently, with a heteroaromatic compound,
or
X and R² together form a C₃-C₁₀-cycloalkyl ring, which optionally can contain one or more heteroatoms and optionally can be substituted in one or more places with hydroxy, C₁-C₆-alkyl, C₁-C₆-alkoxy or halogen,
A and B, in each case independently of one another, stand for hydrogen, hydroxy, C₁-C₃-alkyl, C₁-C₆-alkoxy or for the group —SR⁷, —S(O)R⁷, —SO₂R⁷, —NHSO₂R⁷, —CH(OH)R⁷, —CR⁷(OH)—R⁷, C₁-C₆-alkylP(O)OR³OR⁴, —COR⁷ or for 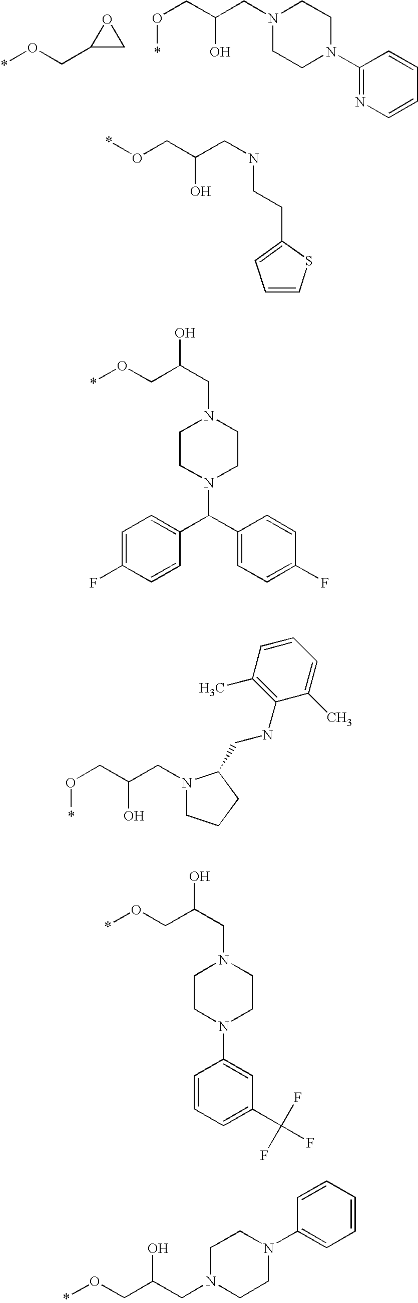 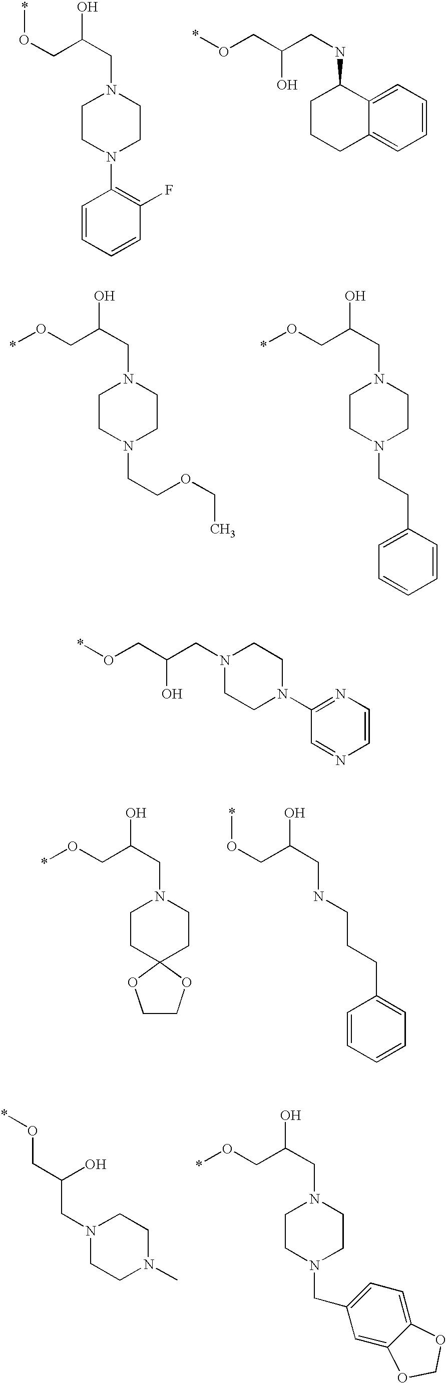 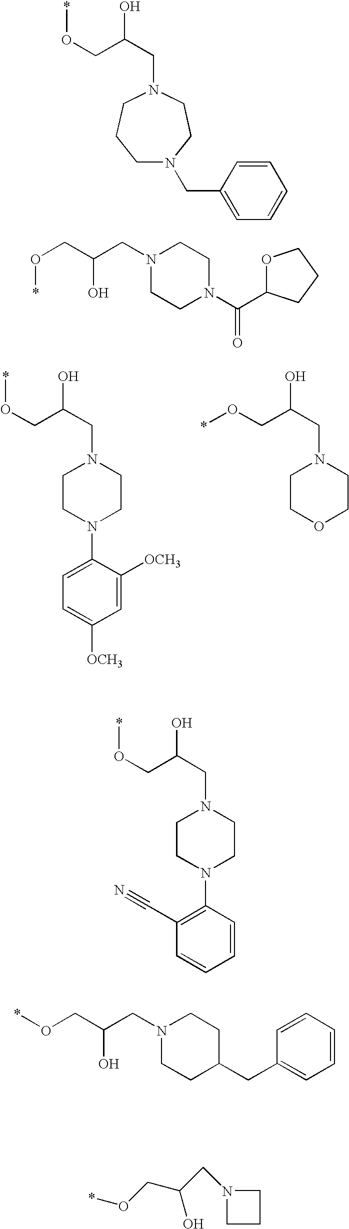
A and B together form a C₃-C₁₀-cycloalkyl ring that optionally can be interrupted by one or more nitrogen, oxygen and/or sulfur atoms and/or can be interrupted by one or more ═C═O or ═SO₂ groups in the ring, and/or optionally one or more possible double bonds can be contained in the ring, and the C₃-C₁₀-cycloalkyl ring optionally can be substituted in one or more places, in the same way or differently, with hydroxy, halogen, C₁-C₆-alkoxy, C₁-C₆-alkylthio, amino, cyano, C₁-C₆-alkyl, C₂-C₆-alkenyl, C₃-C₁₀-cycloalkyl, C₁-C₆-alkoxy-C₁-C₆-alkyl, —NHC₁-C₆-alkyl, —N(C₁-C₆-alkyl)₂, —SO(C₁-C₆-alkyl), —SO₂R⁷, C₁-C₆-alkanoyl, —CONR³R⁴, —COR⁵, C₁-C₆-alkylOAc, phenyl, or with the group R⁶, whereby the phenyl itself optionally can be substituted in one or more places, in the same way or differently, with halogen, hydroxy, C₁-C₆-alkyl, C₁-C₆-alkoxy, or with the group —CF₃ or —OCF₃,
R³ and R⁴, in each case independently of one another, stand for hydrogen, phenyl, benzyloxy, C₁-C₁₂-alkyl, C₁-C₆-alkoxy, C₂-C₄-alkenyloxy, C₃-C₆-cycloalkyl, hydroxy, hydroxy-C₁-C₆-alkyl, dihydroxy-C₁-C₆-alkyl, heteroaryl, heterocyclo-C₃-C₁₀-alkyl, heteroaryl-C₁-C₃-alkyl, C₃-C₆-cycloalkyl-C₁-C₃-alkyl that optionally is substituted with cyano, or for C₁-C₆-alkyl that optionally is substituted in one or more places, in the same way or differently, with phenyl, pyridyl, phenyloxy, C₃-C₆-cycloalkyl, C₁-C₆-alkyl or C₁-C₆-alkoxy, whereby the phenyl itself can be substituted in one or more places, in the same way or differently, with halogen, C₁-C₆-alkyl, C₁-C₆-alkoxy or with the group —SO₂NR³R⁴, or for the group —(CH₂)ₙNR³R⁴, —CNHNH₂ or —NR³R⁴ or
R³ and R⁴ together form a C₃-C₁₀-cycloalkyl ring, which optionally can be interrupted by one or more nitrogen, oxygen and/or sulfur atoms, and/or can be interrupted by one or more ═C═O groups in the ring and/or optionally one or more possible double bonds can be contained in the ring,
R⁵ stands for hydroxy, phenyl, C₁-C₆-alkyl, C₃-C₆-cycloalkyl, benzoxy, C₁-C₆-alkylthio or C₁-C₆-alkoxy,
R⁶ stands for a heteroaryl or a C₃-C₁₀-cycloalkyl ring, whereby the ring has the above-indicated meaning,
R⁷ stands for halogen, hydroxy, phenyl, C₁-C₆-alkyl, C₂-C₆-alkenyl, C₂-C₆-alkinyl, C₃-C₁₀-cycloalkyl with the above-indicated meaning, or for the group —NR³R⁴, or for C₁-C₁₀-alkyl, C₂-C₁₀-alkenyl, C₂-C₁₀-alkinyl or C₃-C₁₀-cycloalkyl that is substituted in one or more places, in the same way or differently, with hydroxy, C₁-C₆-alkoxy, halogen, phenyl, —NR³R⁴ or phenyl, which itself can be substituted in one or more places, in the same way or differently, with halogen, hydroxy, C₁-C₆-alkyl, C₁-C₆-alkoxy, halo-C₁-C₆-alkyl, or halo-C₁-C₆-alkoxy, or R⁷ stands for phenyl, which itself can be substituted in one or more places, in the same way or differently, with halogen, hydroxy, C₁-C₆-alkyl or C₁-C₆-alkoxy, halo-C₁-C₆-alkyl, or halo-C₁-C₆-alkoxy,
R⁸, R⁹ and
R¹⁰, in each case independently of one another, stand for hydrogen, hydroxy, C₁-C₁₀-alkyl, C₂-C₁₀-alkenyl, C₂-C₁₀-alkinyl, C₃-C₁₀-cycloalkyl, aryl, heteroaryl or for C₁-C₁₀-alkyl, C₂-C₁₀-alkenyl, C₂-C₁₀-alkinyl or C₃-C₁₀-cycloalkyl that is substituted in one or more places, in the same way or differently, with hydroxy, halogen, C₁-C₆-alkoxy, C₁-C₆-alkylthio, amino, cyano, C₁-C₆-alkyl, —NH—(CH₂)ₙ—C₃-C₁₀-cycloalkyl, C₃-C₁₀-cycloalkyl, C₁-C₆-hydroxyalkyl, C₂-C₆-alkenyl, C₂-C₆-alkinyl, C₁-C₆-alkoxy-C₁-C₆-alkyl, C₁-C₆-alkoxy-C₁-C₆-alkoxy-C₁-C₆-alkyl, —NHC₁-C₆-alkyl, —N(C₁-C₆-alkyl)₂, —SO(C₁-C₆-alkyl), —SO₂(C₁-C₆-alkyl), C₁-C₆-alkanoyl, —CONR³R⁴, —COR⁵, C₁-C₆-alkylOAc, carboxy, aryl, heteroaryl, —(CH₂)ₙ-aryl, —(CH₂)ₙ-heteroaryl, phenyl-(CH₂)ₙ—R⁵, —(CH₂)ₙPO₃(R⁵)₂ or with the group —R⁶ or —NR³R⁴, and the phenyl, C₃-C₁₀-cycloalkyl, aryl, heteroaryl, —(CH₂)ₙ-aryl and —(CH₂)ₙ-heteroaryl itself optionally can be substituted in one or more places, in the same way or differently, with halogen, hydroxy, C₁-C₆-alkyl, C₁-C₆-alkoxy, or with the group —CF₃ or —OCF₃, and the ring of C₃-C₁₀-cycloalkyl and the C₁-C₁₀-alkyl optionally can be interrupted by one or more nitrogen, oxygen and/or sulfur atoms and/or can be interrupted by one or more ═C═O groups in the ring and/or optionally one or more possible double bonds can be contained in the ring, and
n stands for 0-6,
   as well as the isomers, diastereomers, enantiomers and salts thereof that inhibit the CDK II in vivo and in vitro and thus overcome the drawbacks of the known pharmaceutical agents and can be used for the production of a pharmaceutical agent for contraception.

Alkyl is defined in each case as a straight-chain or branched alkyl radical, such as, for example, methyl, ethyl, propyl, isopropyl, butyl, isobutyl, sec.-butyl, tert.-butyl, pentyl, isopentyl, hexyl, heptyl, octyl, nonyl and decyl.

Alkoxy is defined in each case as a straight-chain or branched alkoxy radical, such as, for example, methyloxy, ethyloxy, propyloxy, isopropyloxy, butyloxy, isobutyloxy, sec.-butyloxy, tert.-butyloxy, pentyloxy, isopentyloxy or hexyloxy.

Alkylthio is defined in each case as a straight-chain or branched alkylthio radical, such as, for example, methylthio, ethylthio, propylthio, isopropyithio, butylthio, isobutylthio, sec.-butylthio, tert.-butylthio, pentylthio, isopentylthio or hexylthio.

In general, cycloalkyl is defined as monocyclic alkyl rings such as cyclopropyl, cyclobutyl, cyclopentyl, cyclohexyl, cycloheptyl, cyclooctyl, cyclononyl or cyclodecyl, but also bicyclic rings or tricyclic rings, such as, for example, norbornyl, adamantanyl, etc.

Ring systems, in which optionally one or more possible double bonds can be contained in the ring, are defined as, for example, cycloalkenyls, such as cyclopropenyl, cyclobutenyl, cyclopentenyl, cyclohexeny, or cycloheptenyl, whereby the linkage can be carried out both to the double bond and to the single bonds.

If A and B, R³ and R⁴, X and R², in each case independently of one another, together form a C₃-C₁₀-cycloalkyl ring, which optionally can be interrupted by one or more heteroatoms such as nitrogen atoms, oxygen atoms and/or sulfur atoms, and/or can be interrupted by one or more ═C═O groups in the ring, and/or optionally one or more possible double bonds can be contained in the ring, however, the above-mentioned definitions are also intended to include the heteroaryl radical or heterocycloalkyl and heterocycloalkenyl.

Halogen is defined in each case as fluorine, chlorine, bromine or iodine.

The alkenyl substituents in each case are straight-chain or branched, whereby, for example, the following radicals are meant: vinyl, propen-1-yl, propen-2-yl, but-1-en-1-yl, but-1-en-2-yl, but-2-en-1-yl, but-2-en-2-yl, 2-methyl-prop-2-en-1-yl, 2-methyl-prop-1-en-1-yl, but-1-en-3-yl, ethinyl, prop-1-in-1-yl, but-1-in-1-yl, but-2-in-1-yl, but-3-en-1-yl, or allyl.

Alkinyl is defined in each case as a straight-chain or branched alkinyl radical that contains 2-6 C atoms, preferably 2-4 C atoms. For example, the following radicals can be mentioned: acetylene, propin-1-yl, propin-3-yl, but-1-in-1-yl, but-1-in-4-yl, but-2-in-1-yl, but-1-in-3-yl, etc.

In each case, the aryl radical comprises 3-12 carbon atoms and can in each case be benzocondensed.

For example, there can be mentioned: cyclopropenyl, cyclopentadienyl, phenyl, tropyl, cyclooctadienyl, indenyl, naphthyl, azulenyl, biphenyl, fluorenyl, anthracenyl, etc.

In each case, the heteroaryl radical comprises 3-16 ring atoms and, instead of carbon, can contain one or more of the same or different heteroatoms, such as oxygen, nitrogen or sulfur in the ring and can be monocyclic, bicyclic or tricyclic, and can in each case in addition be benzocondensed.

For example, there can be mentioned:

Thienyl, furanyl, pyrrolyl, oxazolyl, thiazolyl, imidazolyl, pyrazolyl, isoxazolyl, isothiazolyl, oxadiazolyl, triazolyl, thiadiazolyl, etc., and benzo derivatives thereof, such as, e.g., benzofuranyl, benzothienyl, benzoxazolyl, benzimidazolyl, indazolyl, indolyl, isoindolyl, etc.; or pyridyl, pyridazinyl, pyrimidinyl, pyrazinyl, triazinyl, etc., and benzo derivatives thereof, such as, e.g., quinolyl, isoquinolyl, etc.; or azocinyl, indolizinyl, purinyl, etc., and benzo derivatives thereof; or quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl, oxepinyl, etc.

Heterocycloalkyl stands for an alkyl ring that comprises 3-12 carbon atoms, which instead of carbon contains one or more of the same or different heteroatoms, such as, e.g., oxygen, sulfur or nitrogen.

As heterocycloalkyls, e.g., there can be mentioned: oxiranyl, oxethanyl, aziridinyl, azetidinyl, tetrahydrofuranyl, pyrrolidinyl, dioxolanyl, imidazolidinyl, pyrazolidinyl, dioxanyl, piperidinyl, morpholinyl, dithianyl, thiomorpholinyl, piperazinyl, trithianyl, quinuclidinyl, etc.

Heterocycloalkenyl stands for an alkyl ring that comprises 3-12 carbon atoms, which instead of carbon contains one or more of the same or different heteroatoms, such as, e.g., oxygen, sulfur or nitrogen, and which is partially saturated.

As heterocycloalkenyls, e.g., pyran, thiln, dihydroacet, etc., can be mentioned.

If an acid group is included, the physiologically compatible salts of organic and inorganic bases, such as, for example, the readily soluble alkali salts and alkaline-earth salts, as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropanediol, Sovak base, and 1-amino-2,3,4-butanetriol, are suitable as salts.

If a basic group is included, the physiologically compatible salts of organic and inorganic acids are suitable, such as hydrochloric acid, sulfuric acid, phosphoric acid, citric acid, tartaric acid, i.a.

Those compounds of general formula (I) are especially effective in which
R¹ stands for hydrogen, halogen, C₁-C₆-alkyl, nitro or for the group —COR⁵, —OCF₃, —(CH₂)ₙR⁵, —S—CF₃ or —SO₂CF₃,
R² stands for C₁-C₁₀-alkyl, C₂-C₁₀-alkenyl, C₂-C₁₀-alkinyl or C₃-C₁₀-cycloalkyl or for C₁-C₁₀-alkyl, C₂-C₁₀-alkenyl, C₂-C₁₀-alkinyl or C₃-C₁₀-cycloalkyl that is substituted in one or more places, in the same way or differently, with hydroxy, halogen, C₁-C₆-alkoxy, C₁-C₆-alkylthio, amino, cyano, C₁-C₆-alkyl, —NH—(CH₂)ₙ—C₃-C₁₀-cycloalkyl, C₃-C₁₀-cycloalkyl, C₁-C₆-hydroxyalkyl, C₂-C₆-alkenyl, C₂-C₆-alkinyl, C₁-C₆-alkoxy-C₁-C₆-alkyl, C₁-C₆-alkoxy-C₁-C₆-alkoxy-C₁-C₆-alkyl, —NHC₁-C₆-alkyl, —N(C₁-C₆-alkyl)₂, —SO(C₁-C₆-alkyl), —SO₂(C₁-C₆-alkyl), C₁-C₆-alkanoyl, —CONR³R⁴, —COR⁵, C₁-C₆-alkylOAc, carboxy, aryl, heteroaryl, —(CH₂)ₙ-aryl, —(CH₂)ₙ-heteroaryl, phenyl-(CH₂)ₙ-R⁵, —(CH₂)ₙPO₃(R⁵)₂ or with the group —R⁶ or —NR³R⁴, and the phenyl, C₃-C₁₀-cycloalkyl, aryl, heteroaryl, —(CH₂)ₙ-aryl and —(CH₂)ₙ-heteroaryl itself optionally can be substituted in one or more places, in the same way or differently, with halogen, hydroxy, C₁-C₆-alkyl, C₁-C₆-alkoxy, heteroaryl, benzoxy or with the group —CF₃ or —OCF₃, and the ring of C₃-C₁₀-cycloalkyl and the C₁-C₁₀-alkyl optionally can be interrupted by one or more nitrogen, oxygen and/or sulfur atoms, and/or can be interrupted by one or more ═C═O groups in the ring and/or optionally one or more possible double bonds can be contained in the ring, or
R² stands for the group 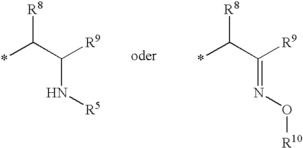
X stands for oxygen or for the group —NH—, —N(C₁-C₃-alkyl) or for —OC₃—C₁₀-cycloalkyl that can be substituted in one or more places, in the same way or differently, with a heteroaromatic compound,
or
X and R² together form a C₃-C₁₀-cycloalkyl ring, which optionally can contain one or more heteroatoms and optionally can be substituted in one or more places with hydroxy, C₁-C₆-alkyl, C₁-C₆-alkoxy or halogen,
A and B, in each case independently of one another, stand for hydrogen, hydroxy, C₁-C₃-alkyl, C₁-C₆-alkoxy or for the group —S—CH₃, —SO₂—C₂H₄—OH, —CO—CH₃, —S—CHF₂, —S—(CH₂)ₙ—CH(OH)CH₂N—R³R⁴, —CH₂P(O)OR³OR⁴, —S—CF₃, —SO—CH₃, —SO₂CF₃, —SO₂—(CH₂)ₙ—N—R³R⁴, —SO₂—NR³R⁴, —SO₂R⁷, —CH—(OH)—CH₃ or for 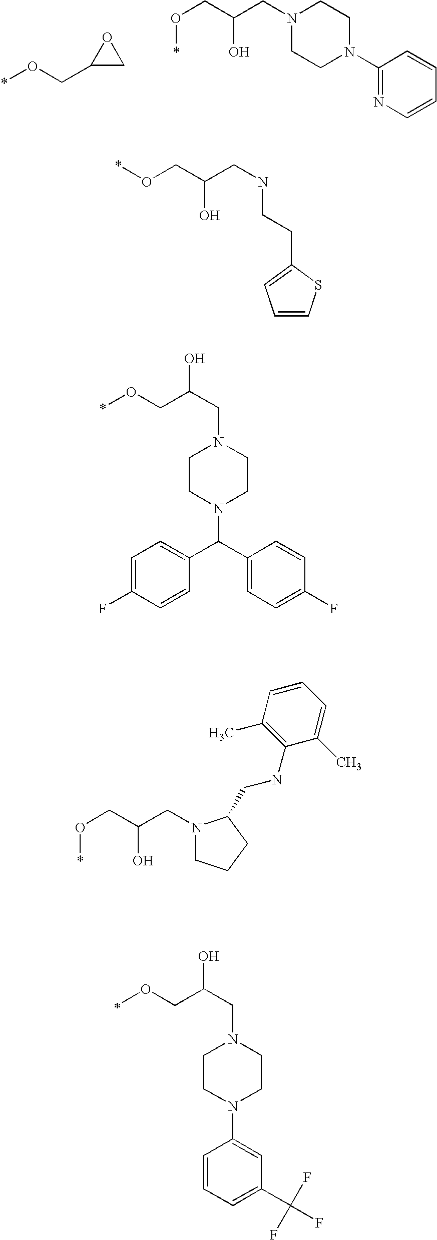 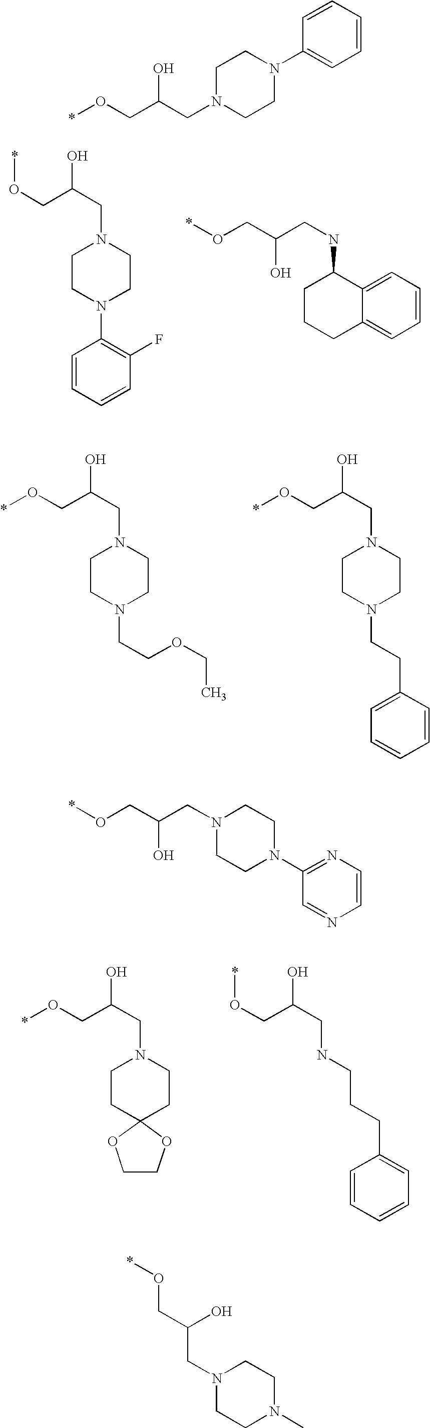 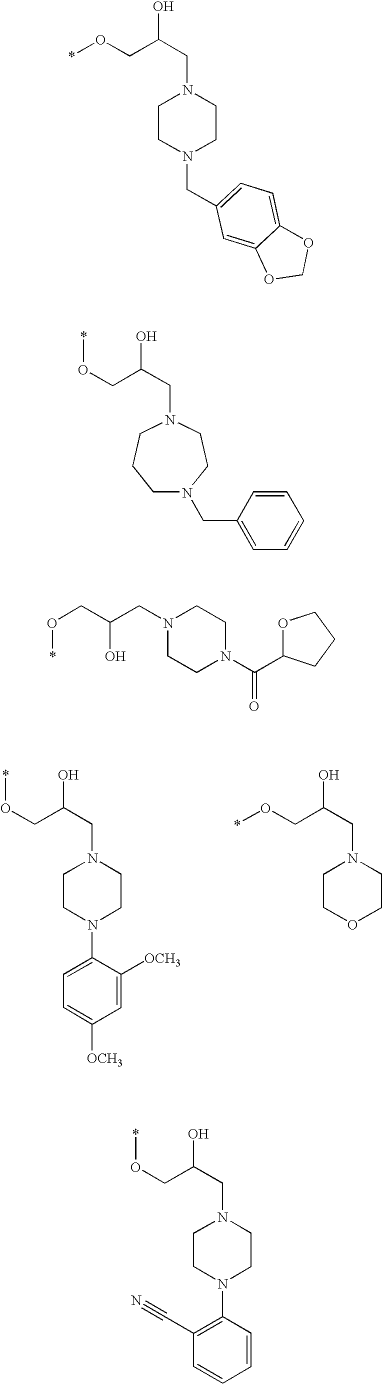 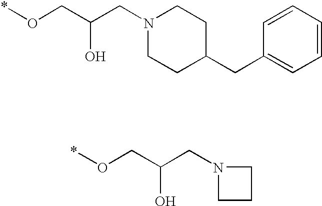
   or
A and B together can form a group 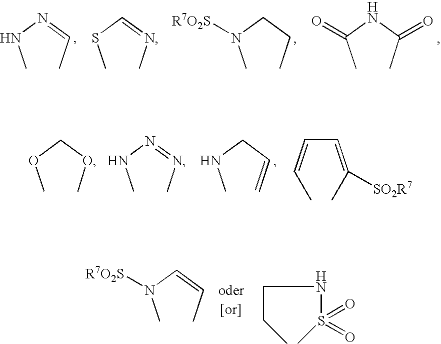
R³ and R⁴, in each case, independently of one another, stand for hydrogen, phenyl, benzyloxy, C₁-C₁₂-alkyl, C₁-C₆-alkoxy, C₂-C₄-alkenyloxy, C₃-C₆-cycloalkyl, hydroxy, hydroxy-C₁-C₆-alkyl, dihydroxy-C₁-C₆-alkyl, heteroaryl, heterocyclo-C₃-C₁₀-alkyl, heteroaryl-C₁-C₃-alkyl, C₃-C₆-cycloalkyl-C₁-C₃-alkyl that optionally is substituted with cyano, or for C₁-C₆-alkyl that optionally is substituted in one or more places, in the same way or differently, with phenyl, pyridyl, phenyloxy, C₃-C₆-cycloalkyl, C₁-C₆-alkyl or C₁-C₆-alkoxy, whereby the phenyl itself can be substituted in one or more places in the same way or differently with halogen, trifluoromethyl, C₁-C₆-alkyl, C₁-C₆-alkoxy or with the group —SO₂NR³R⁴,
   or for the group —(CH₂)ₙNR³R⁴, —CNHNH₂ or —NR³R⁴ or for 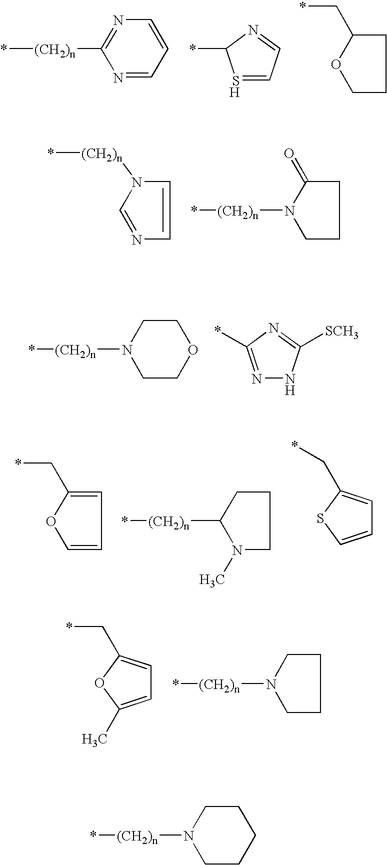
      which optionally can be substituted with C₁-C₆-alkyl,
R⁵ stands for hydroxy, phenyl, C₁-C₆-alkyl, C₃-C₆-cycloalkyl, benzoxy, C₁-C₆-alkylthio or C₁-C₆-alkoxy,
R⁶ stands for the group 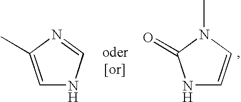
R⁷ stands for halogen, hydroxy, phenyl, C₁-C₆-alkyl, —C₂H₄OH, —NR³R⁴, or for the group 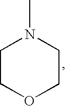
R⁸, R⁹ and
R¹⁰ in each case independently of one another, stand for hydrogen, hydroxy, C₁-C₆-alkyl, C₃-C₆-cycloalkyl or for the group 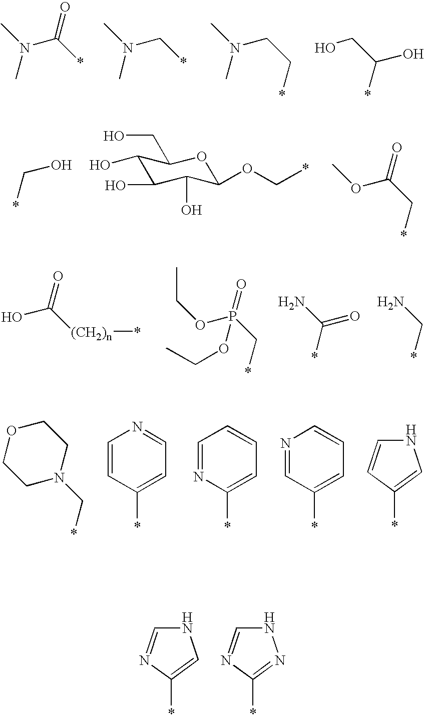
   and
n stands for 0-6, as well as the isomers, enantiomers, diastereomers and salts thereof.

Those compounds of general formula I, in which
R¹ stands for hydrogen, halogen, C₁-C₃-alkyl or for the group —(CH₂)ₙR⁵ steht,
R² stands for —CH(CH₃)—(CH₂)ₙ—R⁵, —CH—(CH₂OH)₂, —(CH₂)ₙR⁷, —CH(C₃H₇)—(CH₂)ₙ—R5, —CH(C₂H₅)—(CH₂)ₙ—R⁵, —CH₂—CN, —CH(CH₃)COCH₃, —CH(CH₃)—C(OH)(CH₃)₂, —CH(CH(OH)CH₃)OCH₃, —CH(C₂H₅)CO—R⁵, C₂-C₄-alkinyl, —(CH₂)ₙ—COR⁵, —(CH₂)ₙ—CO—C₁-C₆-alkyl, —(CH₂)ₙ—C(OH)(CH₃)-phenyl, —CH(CH₃)—C(CH₃)—R⁵, —CH(CH₃)—C(CH₃)(C₂H₅)—R⁵, —CH(OCH₃)—CH₂—R⁵, —CH₂—CH(OH)—R⁵, —CH(OCH₃)—CHR⁵—CH₃, —CH(CH₃)—CH(OH)—CH₂—CH═CH₂, —CH(C₂H₅)—CH(OH)—(CH₂)ₙ—CH₃, —CH(CH₃)—CH(OH)—(CH₂)ₙ—CH₃, —CH(CH₃)—CH(OH)—CH(CH₃)₂, (CH₂OAC)₂, —(CH₂)ₙ—R⁶, —(CH₂)ₙ—(CF₂)ₙ—CF₃, —CH(CH₂)ₙ—R⁵)₂, —CH(CH₃)—CO—NH₂, —CH(CH₂OH)-phenyl, —CH(CH₂OH)—CH(OH)—(CH₂)ₙR⁵, —CH(CH₂OH)—CH(OH)-phenyl, —CH(CH₂OH)—C₂H₄—R⁵, —(CH₂)ₙ—C≡C—C(CH₃)═CH—COR⁵, —CH(Ph)—(CH₂)ₙ—R⁵, —(CH₂)ₙ—COR⁵, —(CH₂)ₙPO₃(R⁵)₂, —(CH₂)ₙ—COR⁵, —CH((CH₂)ₙOR⁵)CO—R⁵, —(CH₂)ₙCONHCH((CH₂)ₙR⁵)₂, —(CH₂)ₙNH—COR⁵, —CH(CH₂)ₙR⁵—(CH₂)ₙC₃-C₁₀-cycloalkyl, —(CH₂)ₙ—C₃-C₁₀-cycloalkyl, C₃-C₁₀-cycloalkyl; C₁-C₆-alkyl that optionally is substituted in one or more places, in the same way or differently, with hydroxy, C₁-C₆-alkyl or the group —COONH(CH₂)ₙCH₃ or —NR³R⁴; C₃-C₁₀-cycloalkyl, —(CH₂)ₙ—O—(CH₂)ₙ—R⁵, —(CH₂)ₙ—NR³R⁴, —CH(C₃H₇)—(CH₂)ₙ—OC(O)—(CH₂)ₙ—CH₃, —(CH₂)ₙ—R⁵, —C(CH₃)₂—(CH₂)ₙ—R⁵—C(CH₂)ₙ(CH₃)—(CH₂)ₙR⁵, —C(CH₂)ₙ—(CH₂)ₙR⁵, —CH(t-butyl)-(CH₂)ₙ—R⁵, —CCH₃(C₃H₇)—(CH₂)ₙR⁵, —CH(C₃H₇)—(CH₂)ₙ—R⁵, —CH(C₃H₇)—COR⁵, —CH(C₃H₇)—(CH₂)ₙ—OC(O)—NH—Ph, —CH((CH₂)ₙ(C₃H₇))—(CH₂)ₙR⁵, —CH(C₃H₇)—(CH₂)ₙ—OC(O)—NH—Ph(OR⁵)₃, —NR³R⁴, —NH—(CH₂)ₙ—NR³R⁴, R⁵—(CH₂)ₙ—C*H—CH(R⁵)—(CH₂)ₙ—R⁵, —(CH₂)ₙ—CO—NH—(CH₂)ₙ—CO—R⁵, —OC(O)NH—C₁-C₆-alkyl or —(CH₂)ₙ—CO—NH—(CH₂)ₙ—CH—((CH₂)ₙR⁵)₂,
or for C₃-C₁₀-cycloalkyl, which is substituted with the group 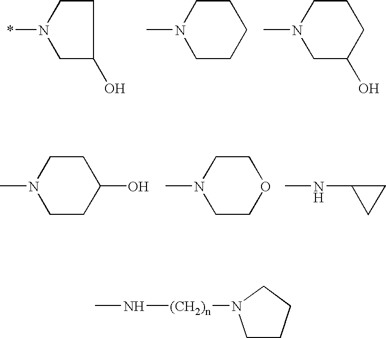
or for the group 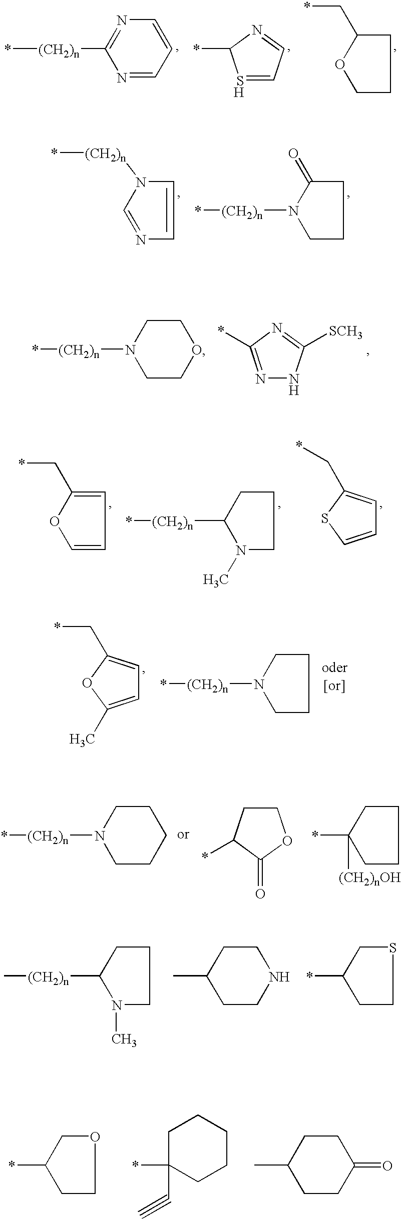
X stands for oxygen or for the group —NH, —N(C₁-C₃-alkyl) or 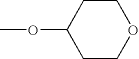
or
R² stands for the group 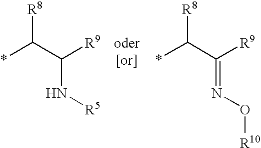
or
X and R² together form a group 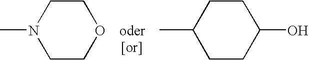
A and B, in each case independently of one another, stand for hydrogen, hydroxy, C₁-C₃-alkyl, C₁-C₆-alkoxy or for the group —S—CH₃, —SO₂—C₂H₄—OH, —CO—CH₃, —S—CHF₂, —S—(CH₂)ₙCH(OH)CH₂N—R³R⁴, —CH₂PO(OC₂H₅)₂, —S—CF₃, —SO—CH₃, —SO₂CF₃, —SO₂—(CH₂)ₙ—N—R³R⁴, —SO₂—NR³R⁴, —SO₂R⁷, —CH(OH)—CH₃, —COOH, —CH((CH₂)ₙR⁵)₂, —(CH₂)ₙR⁵, —COO—C₁-C₆-alkyl, —CONR³R⁴ or for 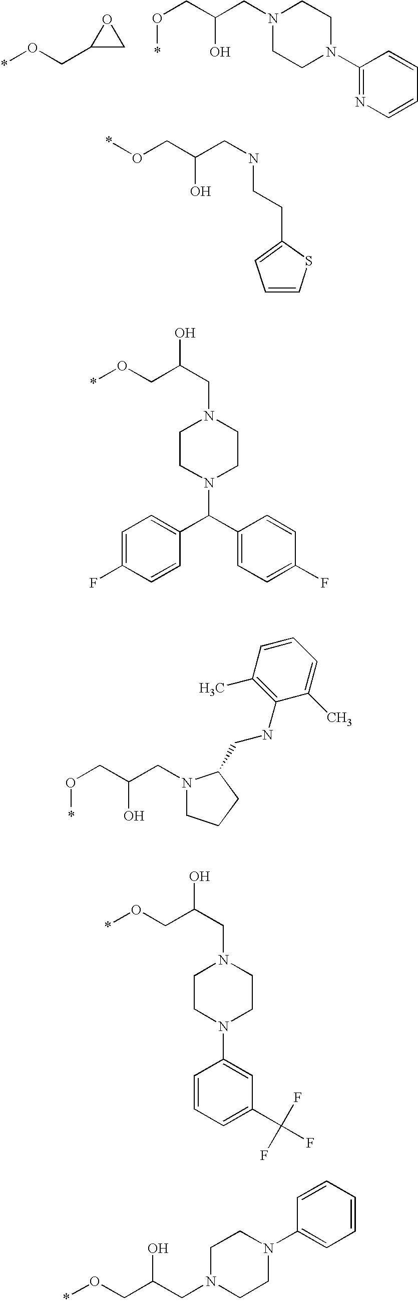 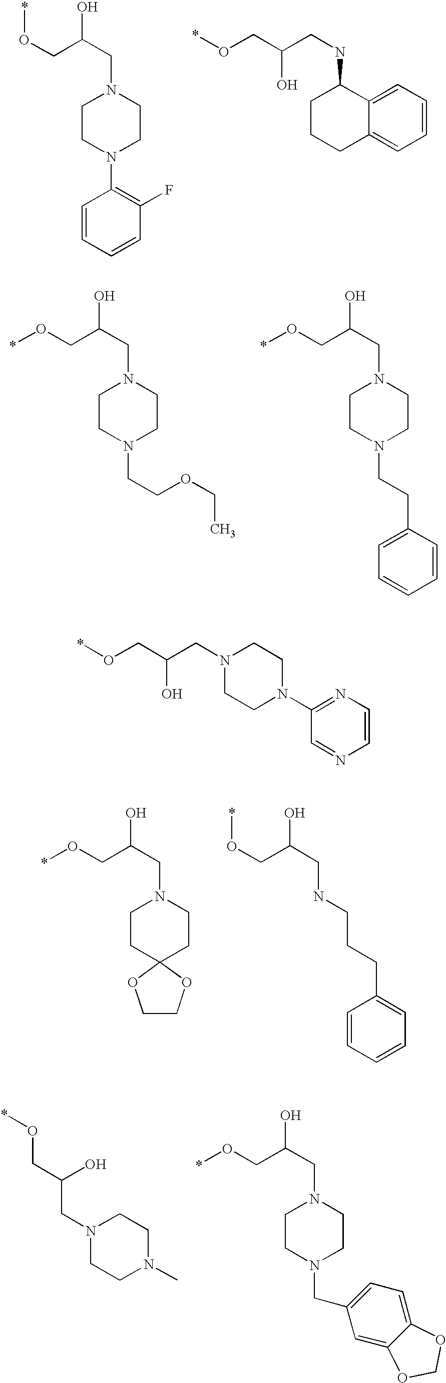 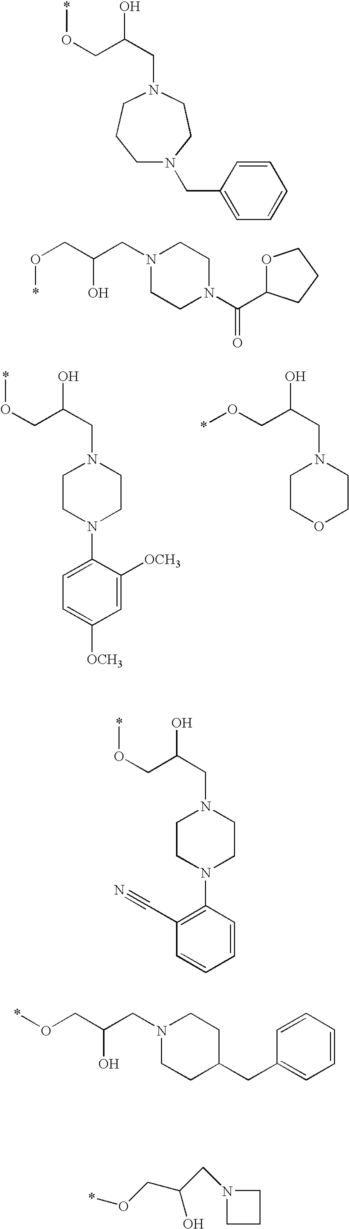
or
A and B together can forrn a group 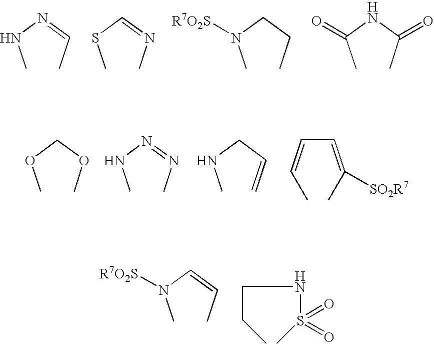
R³ and R⁴, in each case independently of one another, stand for hydrogen, phenyl, benzyloxy, C₁-C₁₂-alkyl, C₁-C₆-alkoxy, C₂-C₄-alkenyloxy, C₃-C₆-cycloalkyl, hydroxy, hydroxy-C₁-C₆-alkyl, dihydroxy-C₁-C₆-alkyl, heteroaryl, heterocyclo-C₃-C₁₀-alkyl, heteroaryl-C₁-C₃-alkyl, C₃-C₆-cycloalkyl-C₁-C₃-alkyl that optionally is substituted with cyano, or for C₁-C₆-alkyl that optionally is substituted in one or more places, in the same way or differently, with phenyl, pyridyl, phenyloxy, C₃-C₆-cycloalkyl, C₁-C₆-alkyl or C₁-C₆-alkoxy, whereby the phenyl itself can be substituted in one or more places, in the same way or differently, with halogen, trifluoromethyl, C₁-C₆-alkyl, C₁-C₆-alkoxy or with the group —SO₂NR³R⁴,
or for the group —(CH₂)ₙNR³R⁴, —CNHNH₂ or —NR³R⁴ or for 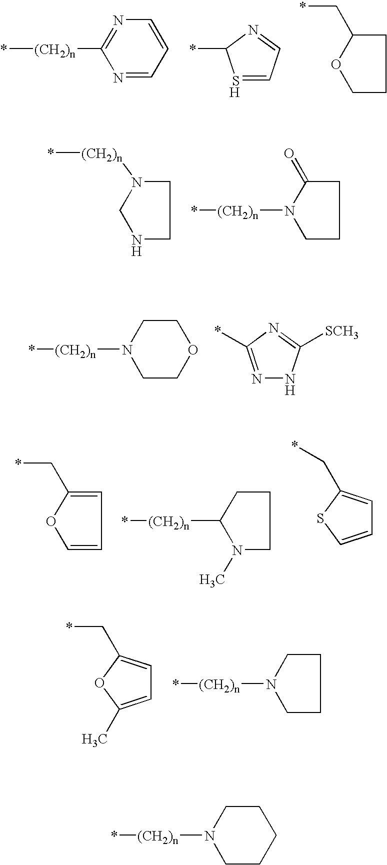
   which optionally can be substituted with C₁-C₆-alkyl,
R⁵ stands for hydroxy, phenyl, C₁-C₆-alkyl, C₃-C₆-cycloalkyl, benzoxy, C₁-C₆-alkylthio or C₁-C₆-alkoxy,
R⁶ stands for the group 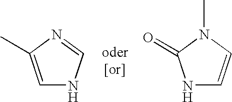
R⁷ stands for halogen, hydroxy, phenyl, C₁-C₆-alkyl, —(CH₂)ₙOH, —NR³R⁴ or the group 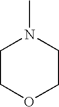
R⁸, R⁹ and
R¹⁰ stand for hydrogen, hydroxy, C₁-C₆-alkyl or for the group —(CH₂)ₙ—COOH, and
n stands for 0-6,
   as well as the isomers, diastereomers, enantiomers and salts thereof, have proven quite especially effective.

The compounds according to the invention inhibit CDK II and thus influence the maturation of male and female germ cells.

A method of birth control comprising the inhibition of CDK II is a subject of this invention.

The use of the compounds of general formulas I, II and IIa for the production of a pharmaceutical agent for the selective modulation of germ cell maturation is a subject of this invention.

The eukaryotic cell division cycle ensures the duplication-of the genome and its distribution to the daughter cells by passing through a coordinated and regulated sequence of events. The cell cycle is divided into four subsequent phases: the GI phase represents the time before the DNA replication in which the cell grows and is sensitive to external stimuli. In the S phase, the cell replicates its DNA, and in the G2 phase, preparations are made for entry into mitosis. In mitosis (M phase), the replicated DNA is separated, and the cell division is completed. (FIG. 1) The cyclin-dependent kinases (CDKs), a family of serine/threonine kinases, whose members require the binding of a cyclin (Cyc) as a regulatory subunit in order for them to activate, drive the cell through the cell cycle. For the progression through the S phase and its ending, the activity of the CDK2/CycE and CDK2/CycA complexes is necessary.

Changes in the cell cycle monitoring play a role in meiosis and thus in the maturation of gametes.

The use of the compounds of general formula I for the production of a pharmaceutical agent for contraception is a subject of this invention.

In this invention, it was also possible to show that the described inhibitors of the CDK II have an influence on the sperm concentration if the substances are administered over a period of 21 days (FIG. 2).

It was also possible to show that the weight of the epididymis is reduced when the substance is administered at a concentration of 25 mg/kg daily (FIG. 3). FIG. 4 shows microscopic images of mouse testes. While the testes of control animals (FIG. 4a) do not show any special morphological characteristics, the testes of animals treated with test substance (FIG. 4b) show a physiologically disturbed epithelium. FIG. 4c shows that in the tail of the epididymis of the animals of the test substance group, spermatides were released prematurely from testicular, tubular lumen and were collected in the tail of the epididymis. It was derived therefrom that CDK II is essential for the completion of the 1^{st} meiosis in male mice.

The use of the compounds of general formulas I, II and IIa for the production of a pharmaceutical agent for the reduction of sperm maturation is a subject of this invention.

In this invention, it was also possible to show that the administration of CDK II inhibitors causes the spontaneous meiotic maturation of mouse egg cells to be inhibited when the test substances are administered at a concentration of 1 μm (FIG. 5).

The use of the compounds of general formulas I, II and IIa for the production of a pharmaceutical agent for inhibiting the egg cell maturation in vitro and in vivo is a subject of this invention.

It was also possible to show that the described substances have an influence on the fertility of female rats. The implantation rate is reduced by substance administration (FIG. 6).

The use of the compounds of general formulas I, II and Ia for the production of a pharmaceutical agent [for] influencing the implantation rate is also a subject of this invention.

Pharmaceutical agents for contraception that contain at least one compound according to general formulas I, II and IIa, as well as pharmaceutical agents with suitable formulation substances and vehicles, are also subjects. ofthis invention.

To use the compounds according to the invention as pharmaceutical agents, the latter are brought into the form of a pharmaceutical preparation, which, in addition to the active ingredient for enteral or parenteral administration, contains suitable pharmaceutical, organic or inorganic inert carrier materials, such as, for example, water, gelatin, gum Arabic, lactose, starch, magnesium stearate, talc, vegetable oils, polylalkylene glycols, etc. The pharmaceutical preparations can be present in solid form, for example as tablets, coated tablets, suppositories, or capsules, or in liquid form, for example as solutions, suspensions or emulsions. Moreover, they optionally contain. adjuvants, such as preservatives, stabilizers, wetting agents, or emulsifiers; salts for changing the osmotic pressure, or buffers.

These pharmaceutical preparations are also subjects of this invention.

For parenteral use, in particular injection solutions or suspensions, in particular aqueous solutions of active compounds in polyhydroxyethoxylated castor oil, are suitable.

As vehicle systems, surface-active adjuvants such as salts of bile acids or animal or plant phospholipids, but also mixtures thereof as well as liposomes or their components can also be used.

For oral use, in particular tablets, coated tablets or capsules with talc and/or hydrocarbon vehicles or binders, such as, for example, lactose, corn or potato starch, are suitable. The use can also be carried out in liquid form, such as, for example, as a juice, to which optionally a sweetener is added.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose to be administered once or divided into two or more daily doses.

If the production of the starting compounds is not described, the latter are known or can be produced analogously to known compounds or to processes that are described here. It is also possible to perform all reactions that are described here in parallel reactors or by means of combinatory operating techniques.

The isomer mixtures can be separated into enantiomers or E/Z isomers according to commonly used methods, such as, for example, crystallization, chromatography or salt formation.

The production of salts is carried out in the usual way by a solution of the compounds of formulas I, II and IIa being mixed with the equivalent amount or an excess of a base or acid, which optionally is in solution, and the precipitate being separated or the solution being worked up in the usual way.

Production of the Compounds According to the Invention

The examples below explain the production of the compounds according to the invention without limiting the scope of the claimed compounds to these examples.

The compounds of general formula I according to the invention can be produced according to the general procedural schemes below:

[image]

[image]

[image]

[Key to Diagram 3:]

geleche Metliode=Same Method

### EXAMPLE 1

### Production of 5-Bromo-N2-(4-difluoromethylthiophenyl)-N4-2-propynyl-2,4-pyrimidinediamine (carried out according to process diagram 1) (compound 23)

245 mg (1 mmol) of 2-chloro-4-2-propynylaminopyrimidine is dissolved in 2 ml of acetonitrile, and a suspension of 4-(difluoromethylthio)-aniline hydrochloride [produced from 352 mg (2 mmol) of 4-(difluoromethylthio)-aniline, 1 ml of acetonitrile and 0.5 ml of aqueous HCl (4 M in dioxane)] is added at room temperature. Then, the reaction mixture is refluxed overnight under an N₂ atmosphere. After cooling, the mixture is filtered, the remaining solid phase is washed with H₂O and dried. A yield of 328 mg (85%) of the product can be expected; the melting point is >235° C.

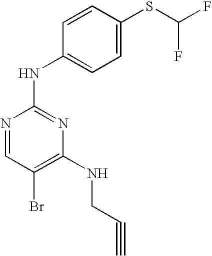

### EXAMPLE 2

### Production of 5-Bromo-N-(3-(oxiranylmethoxy)phenyl)-2-(2-propynyloxy)-2-pyrimidinamine (compound 51), carried out according to process diagram 2

1.55 g (4.9 mmol) of compound 20 is dissolved in 5.5 ml of epibromohydrin, and 1.38 g of K₂CO₃ and 65 mg of tetrabutylammonium bromide are added thereto. The reaction mixture is stirred for 1 hour at 100° C. under nitrogen atmosphere. After ethyl acetate is added, the resulting precipitate is collected and recrystallized from ethanol. The product yield is 1.15 g (62%) as a white powder; the melting point is approximately 173° C.

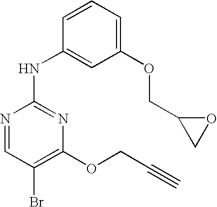

Substance 40 is produced analogously to Example 2:

| | | |
|---|---|---|
| 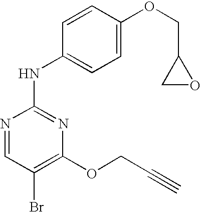 | Chromatography: Yield: Melting Point: | H/EA 1:3 0.5% TEA 38% 140-141° C. |

### EXAMPLE 3

### Production of 1-(4-((5-Bromo-4-(2-propynyloxy)-pyrimidin-2-yl)-amino)phenoxy)-3-(4-phenylpiperazin-1-yl)-2-propanol (Compound 41)

0.2 ml of a 0.5 M 4-phenylpipetazine solution in DMPU is added to a solution of 19 mg (0.05 mmol) of substance 51 in N,N′-dimethylpropylurea (DMPU). The reaction mixture is kept at a temperature of 80° C. for 18 hours. After cooling, 3.5 ml of tert-butyl methyl ether is added, and the organic phase is extracted 5 times with 1.5 ml of H₂O and then evaporated in a vacuum. The remaining residue is chromatographed on 1.7 g (15 pmol) of Lichrosphere Si60 (gradient: dichloromethane/hexane 1:1 to DCM and then dichloromethane/methanol 99:1 to 93:7). A product yield of 17 mg (64%) is achieved.

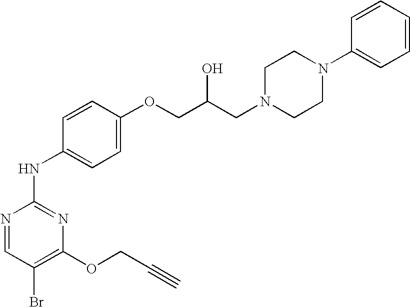

Similarly produced are also the following compounds:

| | |
|---|---|
| 28 | 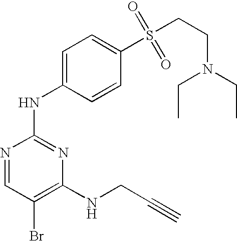 |
| 30 | 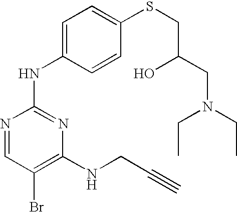 |
| 32 | 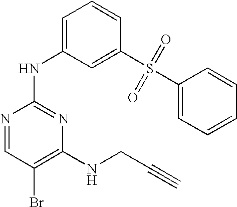 |
| 33 | 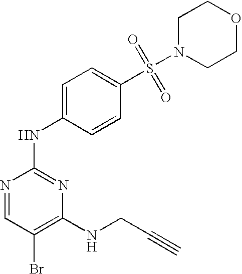 |
| 41 | 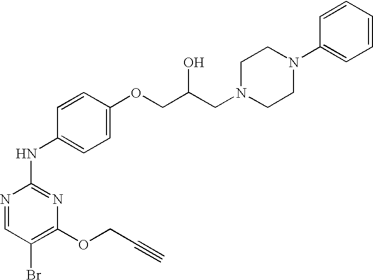 |
| 57 | 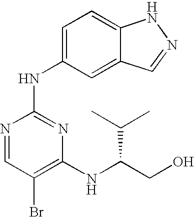 |
| 58 | 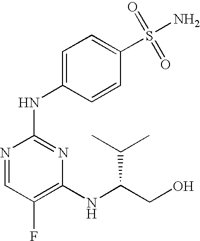 |
| 59 | 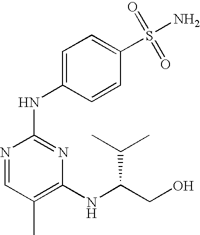 |
| 62 | 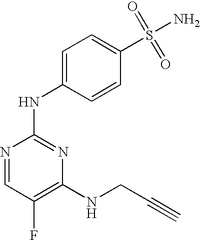 |
| 65 | 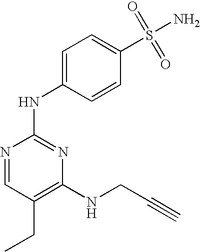 |
| 66 | 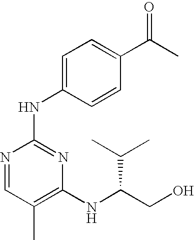 |
| 68 | 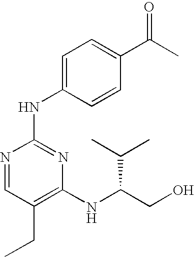 |
| 72 | 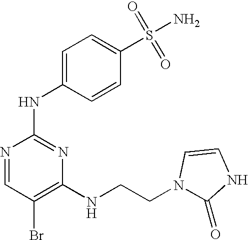 |
| 73 | 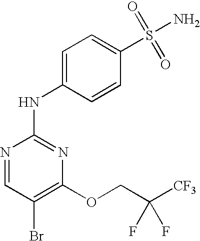 |
| 75 | 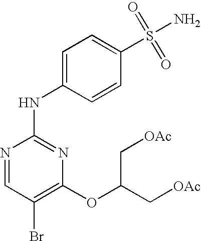 |
| 76 | 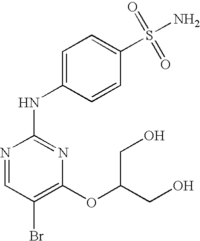 |
| 79 | 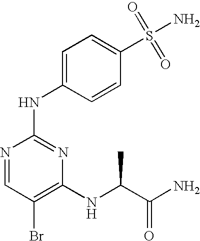 |
| 83 | 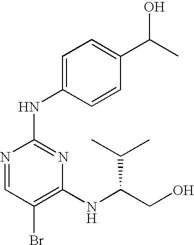 |
| 96 | 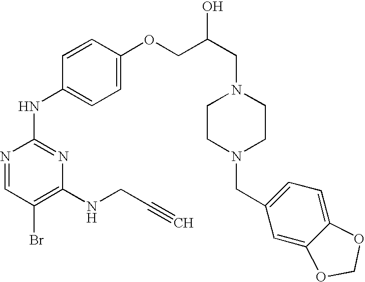 |
| 97 | 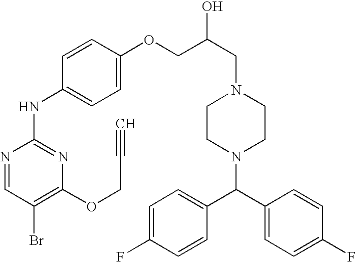 |
| 98 | 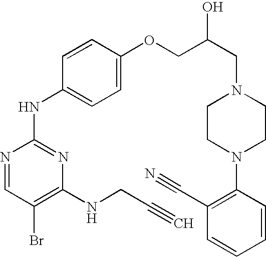 |
| 99 | 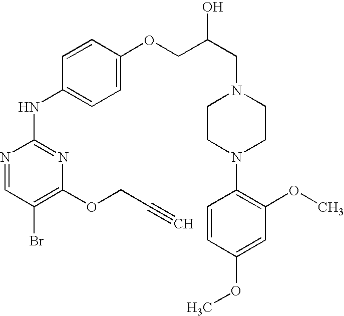 |
| 100 | 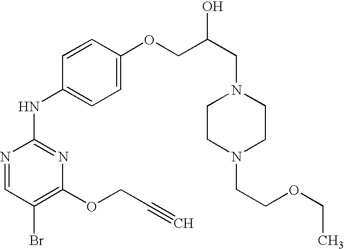 |
| 101 | 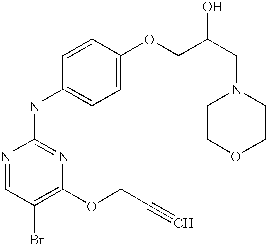 |
| 102 | 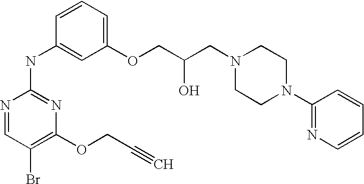 |
| 103 | 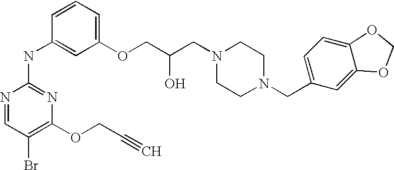 |
| 104 | 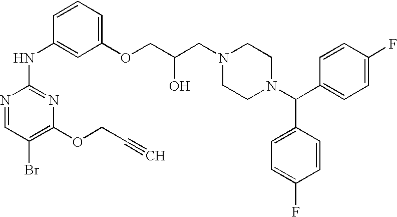 |
| 105 | 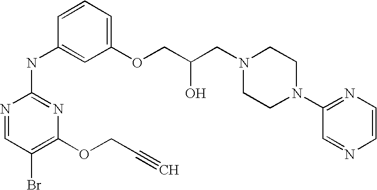 |
| 106 | 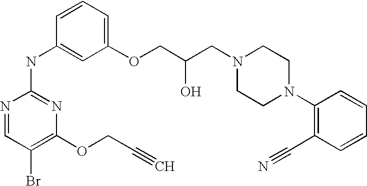 |
| 107 | 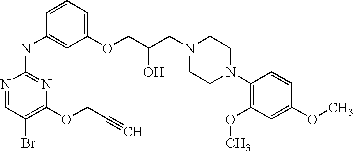 |
| 108 | 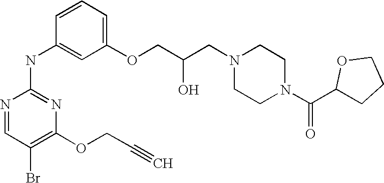 |
| 109 | 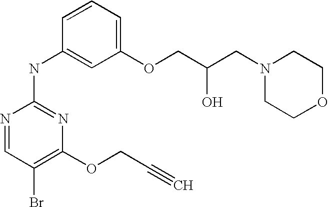 |
| 110 | 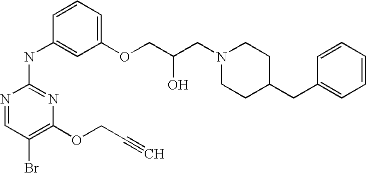 |
| 111 | 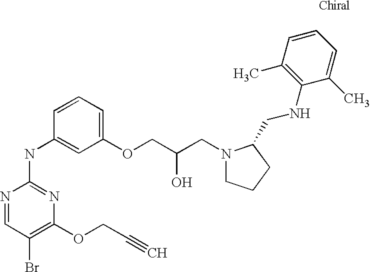 |
| 112 | 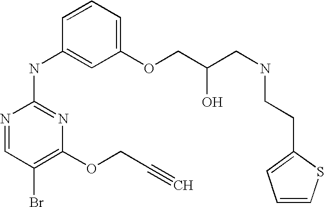 |
| 113 | 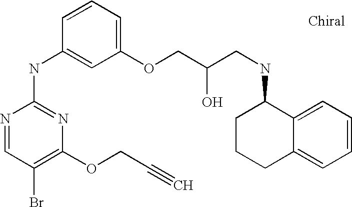 |
| 114 | 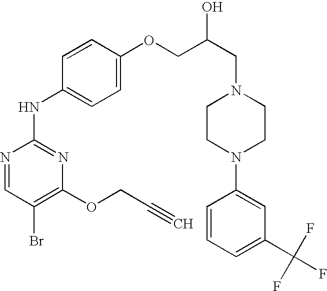 |
| 115 | 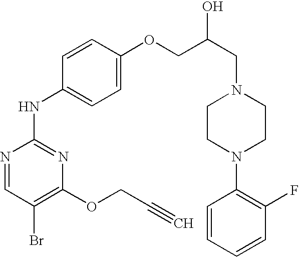 |
| 116 | 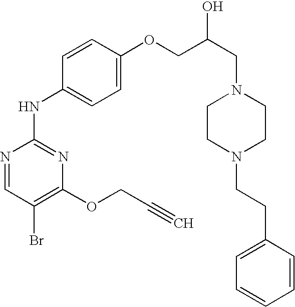 |
| 117 | 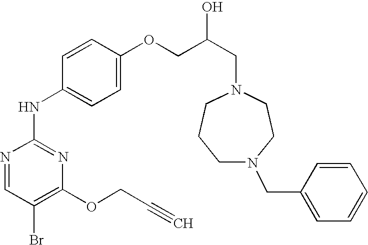 |
| 118 | 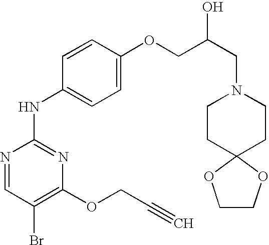 |
| 119 | 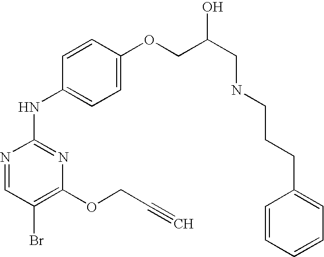 |

The following compounds were produced analogously to the described synthesis processes according to Diagram 1 or 2:

| | 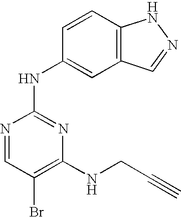 | 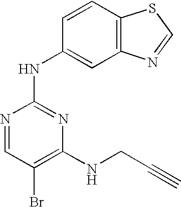 | 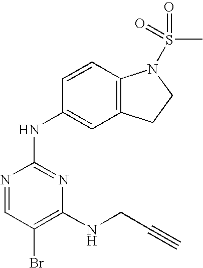 | 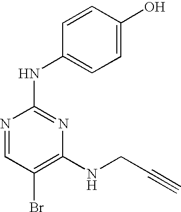 |
|---|---|---|---|---|
| Ex.No. | 37 | 38 | 39 | 5 |
| Chromato- | EA + 0.5% | — | Crystallized | — |
| graphy | TEA | | MeOH | |
| Yield | 10% | 36% | 73% | 20% |
| Melting | 231° C. | >235° C. | 237° C. | 157° C. |
| Point | | | | |
| | 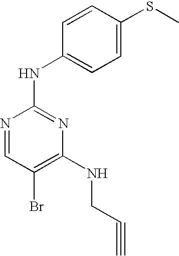 | 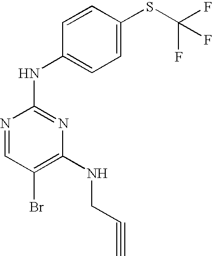 | 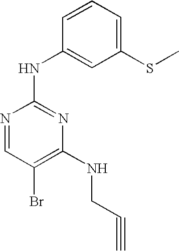 | 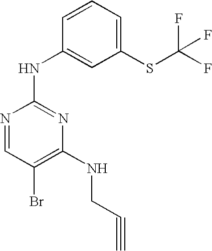 |
| Ex.No. | 16 | 24 | 26 | 35 |
| Chromato- | — | — | — | — |
| graphy | | | | |
| Yield | 94% | 86% | 73% | 69% |
| Melting | 232-234° C. | 160° C. | 194° C. | 143° C. |
| Point | | | | |
| | 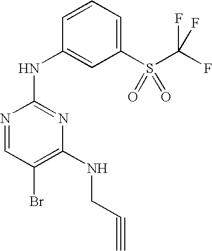 | 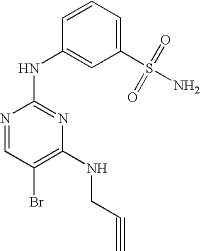 | 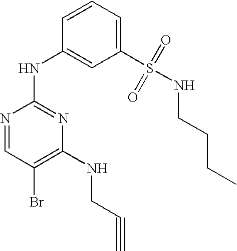 | 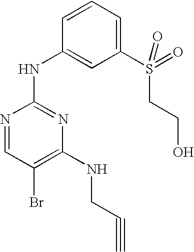 |
| Ex.No. | 27 | 36 | 34 | 21 |
| Chromato- | — | Crystallized | — | — |
| graphy | | EtOH | | |
| Yield | 69% | 64% | 87% | 59% |
| Melting | 144° C. | 219° C. | 220° C. | 192.5-193.5° C. |
| Point | | | | |
| | 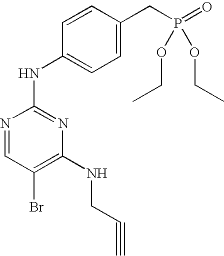 | 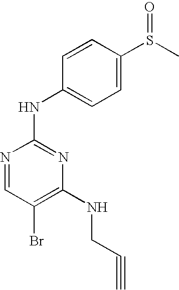 | 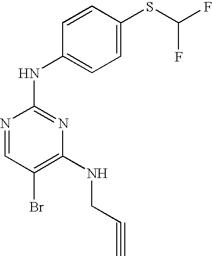 | 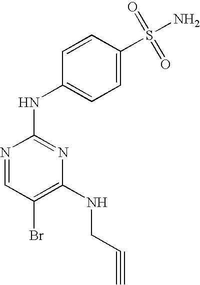 |
| Ex.No. | 31 | 25 | 23 | 11 |
| Chromato- | BA | DCM/MeOH | — | Crystallized |
| graphy | Crystallized | 95:5 | | DIPE/EtOH |
| | H/DIPE | | | |
| Yield | 23% | 25% | 85% | 17% |
| Melting | 198° C. | 217-218° C. | >235° C. | >235° C. |
| Point | | | | |
| | 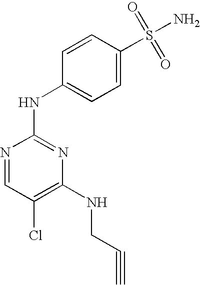 | 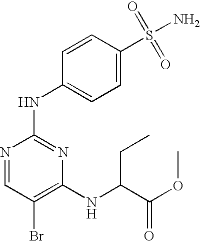 | 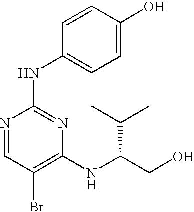 | |
| Ex.No. | 44 | 45 | 4 | |
| Chromato- | Crystallized | Crystallized | — | |
| graphy | EtOH | EtOH | | |
| Yield | 27% | 48% | 52% | |
| Melting | 252° C. | 235° C. | 242-243° C. | |
| Point | | | | |
| | 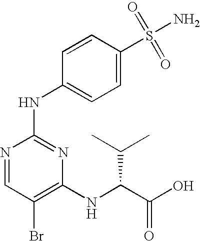 | 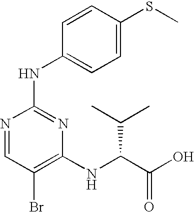 | 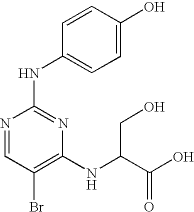 | 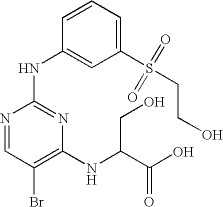 |
| Ex.-No. | 10 | 15 | 3 | 19 |
| Chromato- | — | — | — | Crystallized |
| graphy | | | | Water |
| Yield | 43% | 27% | 76% | 52% |
| Melting | 252-253° C. | 192-193° C. | 257-258° C. | 209-210° C. |
| Point | | | | |
| | 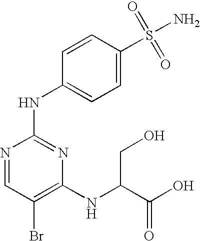 | 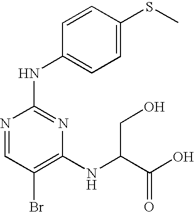 | 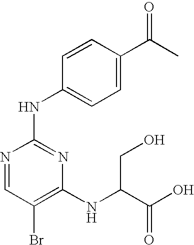 | 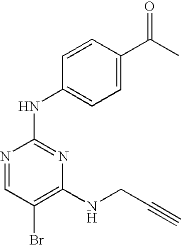 |
| Ex.No. | 9 | 14 | 55 | 50 |
| Chromato- | Crystallized | | Crystallized | — |
| graphy | MeOH | | MeOH/DIPE | |
| Yield | 24% | 91% | 27% | 56% |
| Melting | 247-248° C. | 233-234° C. | 228-229° C. | 241° C. |
| Point | | | | |
| | 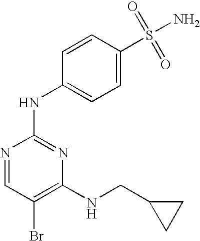 | 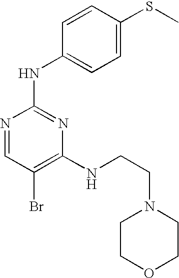 | 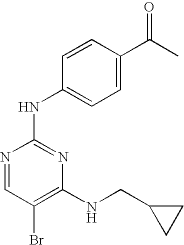 | 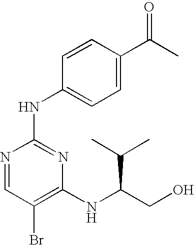 |
| Ex.-No. | 46 | 13 | 52 | 53 |
| Chromato- | H/EA 1:2 | — | — | H/EA 1:2 |
| graphy | | | | |
| Yield | 20% | 28% | 53% | 9% |
| Melting | 256° C. | 185-186° C. | 183° C. | 170° C. |
| Point | | | | |
| | 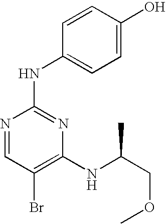 | 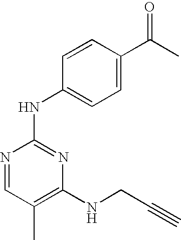 | 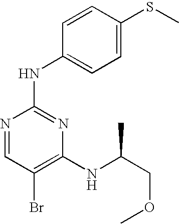 | 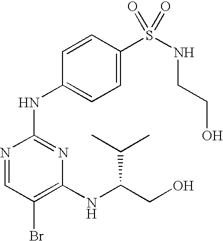 |
| Ex.No. | 1 | 54 | 12 | 60 |
| Chromato- | Crystallized | Crystallized | Crystallized. | |
| graphy | BA | DIPE/MeOH | BA | |
| Yield | 64% | 52% | 36% | |
| Melting | 165.5-166° C. | 210° C. | 91° C. | 150-151° C. |
| Point | | | | |
| | 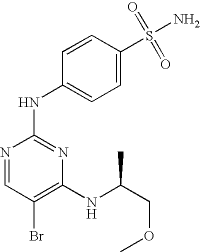 | 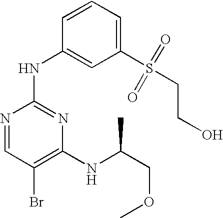 | 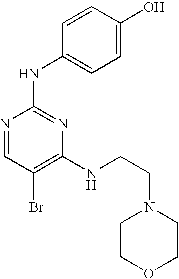 | 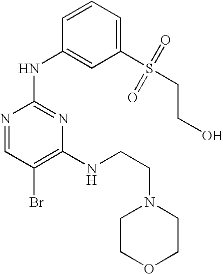 |
| Ex.No. | 7 | 17 | 2 | 18 |
| Chromato- | | — | — | Crystallized |
| graphy | | | | MeOH |
| Yield | 25% | 10% | 62% | 50% |
| Melting | 247° C. | 201-202° C. | 227.5-228.5° C. | 245° C. |
| Point | Decomposi- | | | Decomposition |
| | tion | | | |
| | 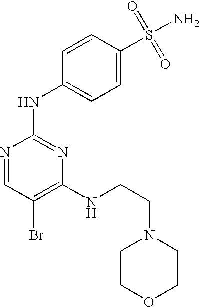 | 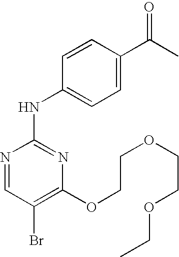 | 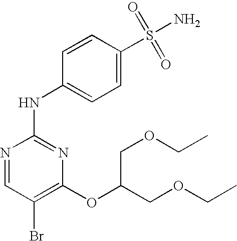 | |
| Ex. No. | 8(D₂O) | 86 | 77 | |
| Chromato- | Crystallized | — | — | |
| graphy | Water | | | |
| Yield | 25% | 2% | 74% | |
| Melting | >275° C. | 85° C. | 132° C. | |
| Point | | | | |
| | 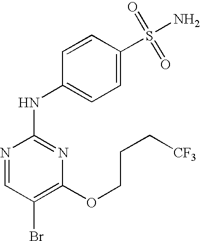 | 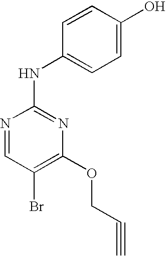 | 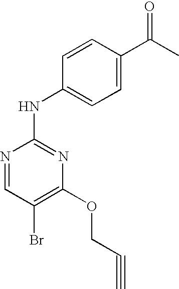 | 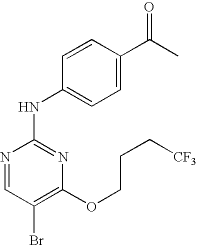 |
| Ex.No. | 47 | 6 | 22 | 84 |
| Chromato- | MeOH/DCM | | — | — |
| graphy | 1:9 | | | |
| Yield | 4% | 66% | 8% | 11% |
| Melting | 186-187° C. | 146° C. | 165-166° C. | 152° C. |
| Point | | | | |
| | 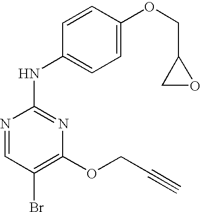 | 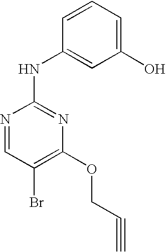 | | |
| Ex.No. | 40 | 20 | | |
| Chromato- | H/EA 1:3 | Crystallized | | |
| graphy | 0.5% TEA | DuPE | | |
| Yield | 38% | 35% | | |
| Melting | 140-141° C. | 174° C. | | |
| Point | | | | |
| | 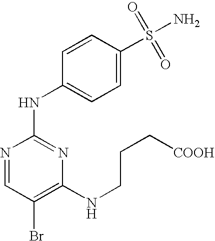 | 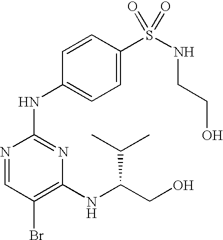 | 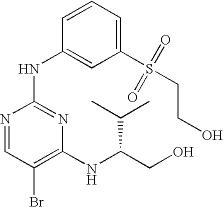 | 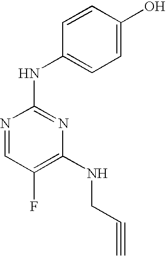 |
| Ex.No. | 49 | 48 | 29 | 42 |
| Chromato- | — | Crystallized | DCM/EA 2:1 | HIEA 1:2 |
| graphy | | MeOH/DIPE | | |
| Yield | 9% | 16% | 26% | 29% |
| Melting | 262° C. | 150-151° C. | | 163° C. |
| Point | | | | |
| | 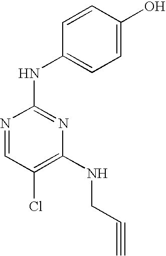 | 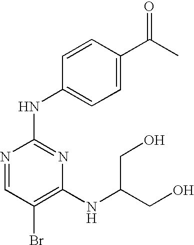 | 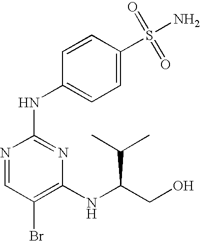 | 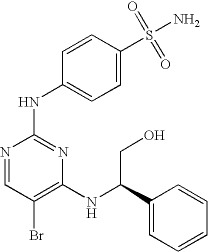 |
| Ex.No. | 43 | 55 | 89 | 88 |
| Chromato- | H/EA 1:2 | Crystallized | — | — |
| graphy | | MeOH/DIPE | | |
| Yield | 35% | 27% | 74% | 27% |
| Melting | 168° C. | 228° C. | 248° C. | 159° C. |
| Point | | | Decomposition | Decomposition |
| | 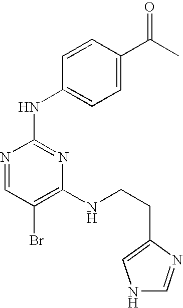 | 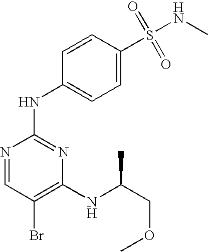 | 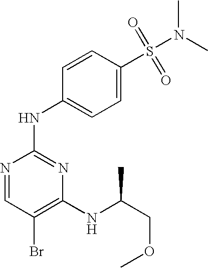 | 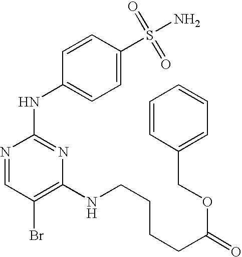 |
| Ex.No. | 87 | 92 | 91 | 96 |
| Chromato- | — | H to H/EA 1:1 | H to H/EA 1:1 | — |
| graphy | | | | |
| Yield | 16% | 21% | 7% | 33% |
| Melting | 210° C. | 167-168° C. | 105° C. | 202° C. |
| Point | | | | |
| | 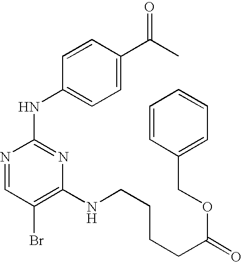 | 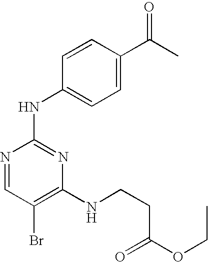 | 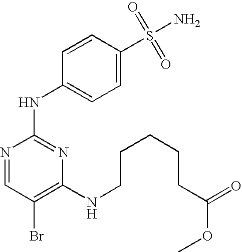 | 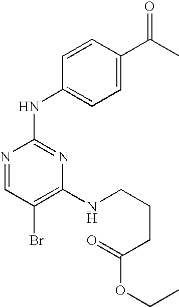 |
| Ex.No. | 97 | 98 | 90 | 85 |
| Chromato- | — | — | | — |
| graphy | | | | |
| Yield | 23% | 32% | | 53% |
| Melting | 152° C. | 172 | | 184° C. |
| Point | | | | |
| | 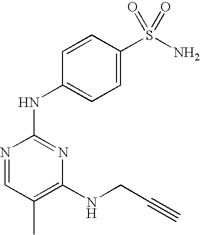 | 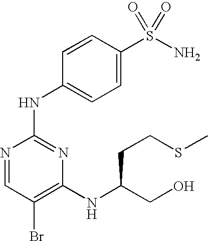 | 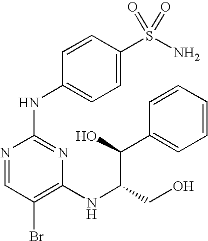 | 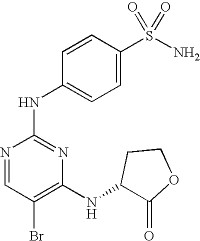 |
| Ex.No. | 63 | 94 | 93 | 80 |
| Yield | 61% | 24% | 70% | 51% |
| Melting | 220° C. | 168° C. | 243° C. | |
| Point | | | | |
| Mass | 428 (EI) | 462 (ES) | 494 (ES) | 427 (EI) |
| | 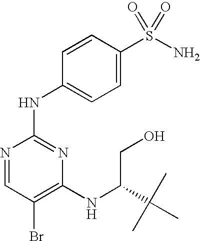 | 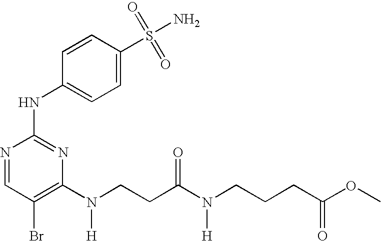 | 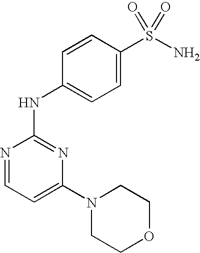 | 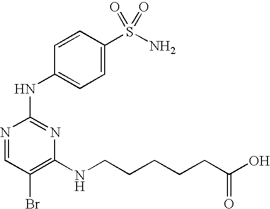 |
| Ex.No. | 120 | 121 | 122 | 123 |
| Yield | 49% | 24% | 80% | 73% |
| Melting | 252° C. | | | |
| Point | | | | |
| Mass | 445 (EI) | 516 (EI) | 334 (EI) | 459 (EI) |
| | 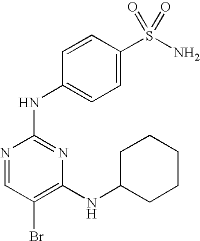 | 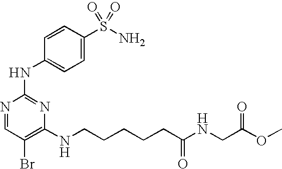 | 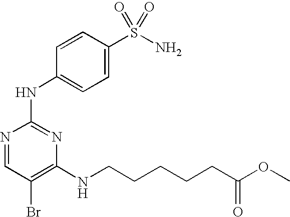 | 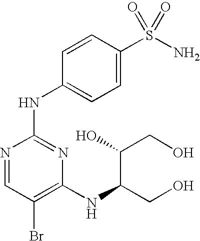 |
| Ex.No. | 95 | 124 | 125 | 126 |
| Yield | 29% | 25% | 27% | 46% |
| Melting | 255° C. | | 218° C. | |
| Point | | | | |
| Mass | 425 (EI) | 557 (ES) | 471 (EI) | 449 (EI) |
| | 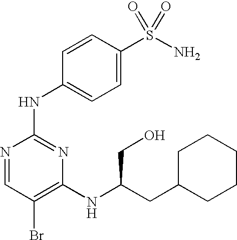 | 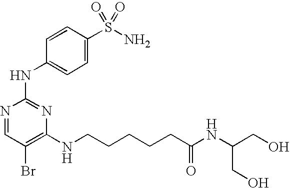 | 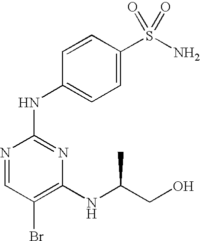 | 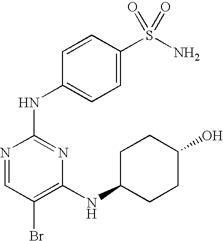 |
| Ex.No. | 127 | 128 | 129 | 130 |
| Yield | 18% | 94% | 61% | 58% |
| Melting | 220° C. | | 259° C. | 262° C. |
| Point | | | | |
| Mass | 485 (EI) | 531 (ES) | 403 (EI) | 443 (EI) |
| | 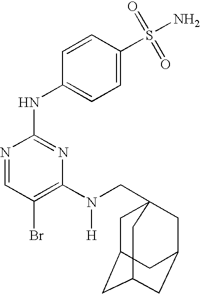 | 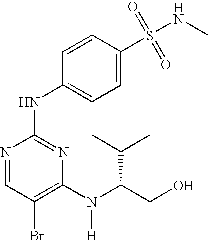 | 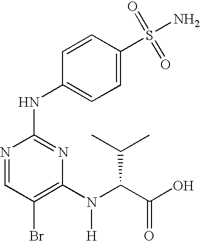 | 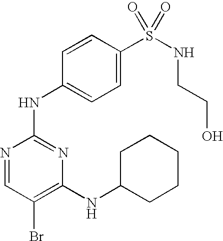 |
| Ex.No. | 131 | 132 | 133 | 134 |
| Yield | 9% | 42% | 25% | 64% |
| Melting | 229° C. | 141° C. | | |
| Point | | | | |
| Mass | 491 (EI) | 443 (EI) | 444 (FAB) | |
| | 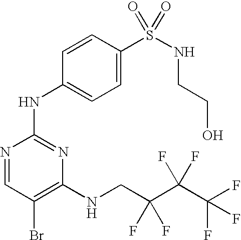 | 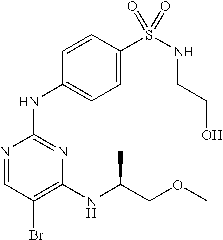 | 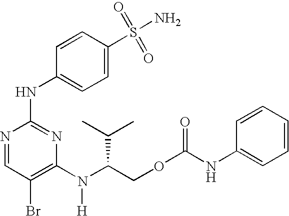 | 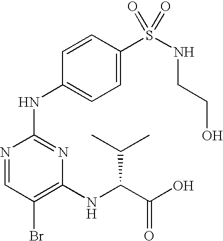 |
| Ex.No. | 135 | 136 | 137 | 138 |
| Yield | 34% | 53% | 59% | 57% |
| Melting | | | | |
| Point | | | | |
| Mass | 570 (ES) | 460 (ES) | 549 (ES) | 488 (ES) |
| | 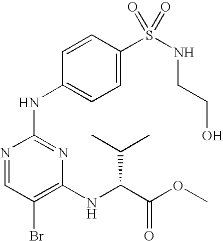 | 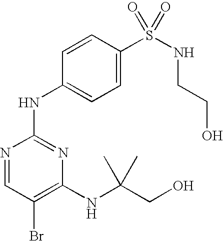 | 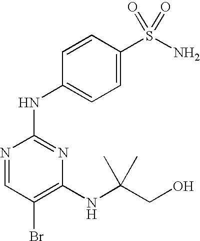 | 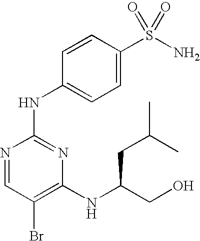 |
| Ex.No. | 139 | 140 | 141 | 142 |
| Yield | 20% | 63% | 23% | 8% |
| Melting | | | | |
| Point | | | | |
| Mass | 502 (ES) | 382 (ES) | 415 (EI) | 443 (EI) |
| | 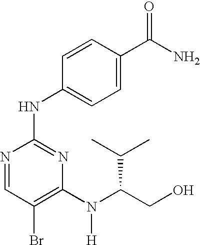 | 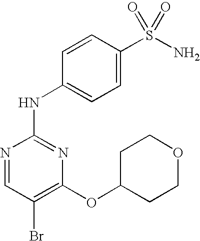 | 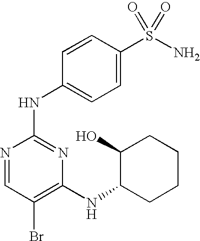 | 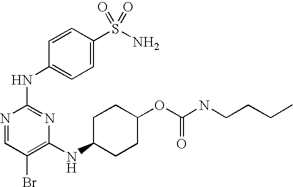 |
| Ex.No. | 143 | 144 | 145 | 78 |
| Yield | 13% | 47% | 42% | 20% |
| Melting | | | | |
| Point | | | | |
| Mass | 392 (EI) | 428 (EI) | 441 (EI) | 541 (ES) |
| | 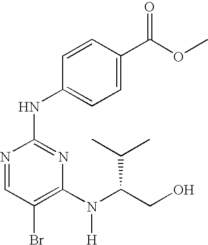 | 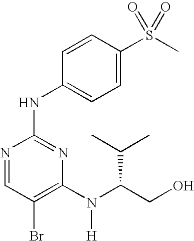 | 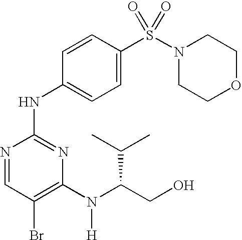 | 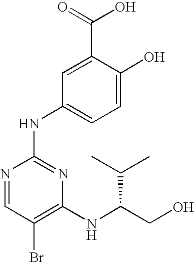 |
| Ex.No. | 146 | 147 | 148 | 149 |
| Yield | 86% | 33% | 79% | 42% |
| Melting | 225° C. | 211° C. | 232° C. | 241° C. |
| Point | | | | |
| Mass | 408 (EI) | 428 (EI) | 501 (EI) | 411 (ES) |
| | 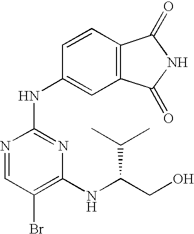 | 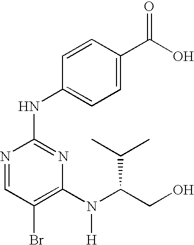 | 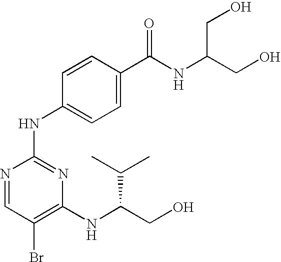 | 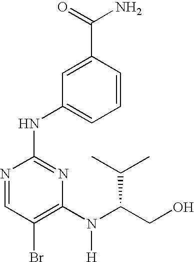 |
| Ex.No. | 150 | 151 | 152 | 153 |
| Yield | 27% | 65% | 85% | 9% |
| Melting | | | | 231° C. |
| Point | | | | |
| Mass | 420 (ES) | 395 (ES) | 468 (ES) | 395 (ES) |
| | 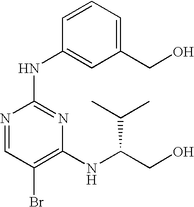 | 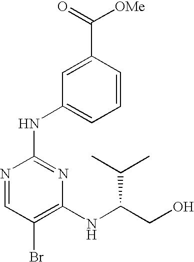 | 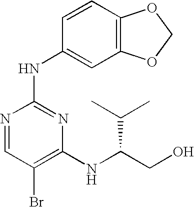 | 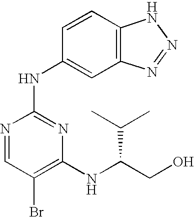 |
| Ex.No. | 154 | 155 | 156 | 157 |
| Yield | 90% | 48% | 77% | 21% |
| Melting | 170° C. | 181° C. | 177° C. | 196° C. |
| Point | | | | |
| Mass | 381 (ES) | 409 (ES) | 394 (EI) | 391 (EI) |
| | 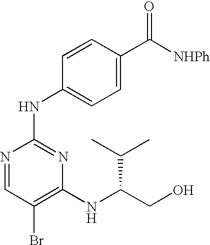 | 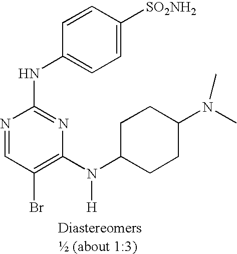 | 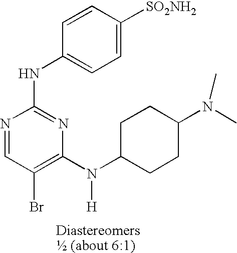 | 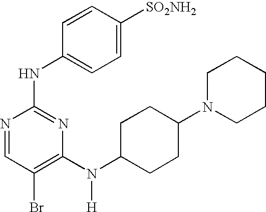 |
| Ex. No. | 158 | 159 * | 160 * | 161 * |
| Yield | 37% | 21% | 14% | 8% |
| Melting | | | 199° C. | >300° C. |
| Point | | | | |
| Mass | 469 (EI) | 468 (EI)468 (EI) | 508 (EI) | |
| | 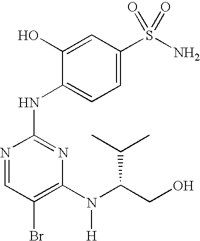 | 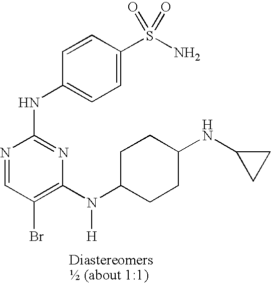 | 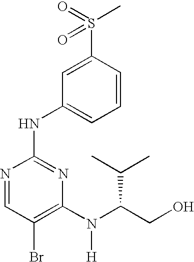 | 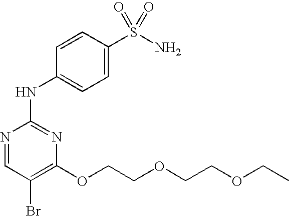 |
| Ex.No. | 162 | 163* | 164 | 165 |
| Yield | 16% | 33% | 14% | 51% |
| Melting | 195° C. | | | 162-164° C. |
| Point | | | | |
| Mass | 446 (ES) | 480 (EI) | 429 (ES) | 462 (EI) |
| | 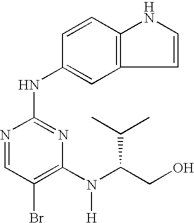 | 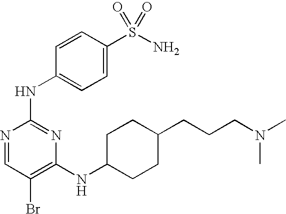 | 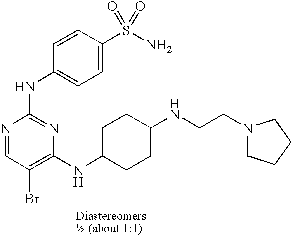 | 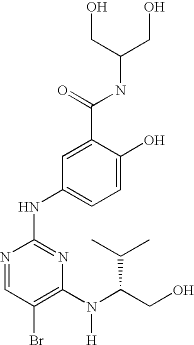 |
| Ex.No. | 166 | 167 * | 168 * | 169 |
| Yield | 6% | 16% | 58% | 60% |
| Melting | | 256° C. | 261° C. | |
| Point | | | | |
| Mass | 390 (ES) | 512 (ES) | 538 (ES) | 484 (ES) |
| | 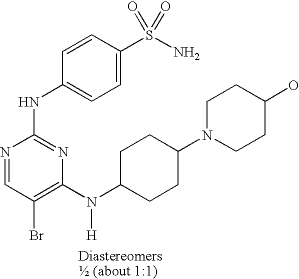 | 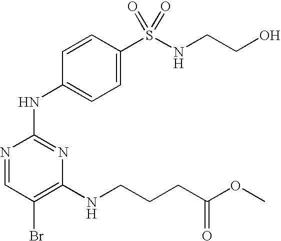 | 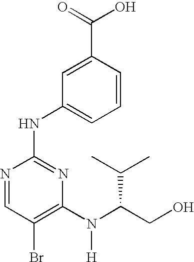 | 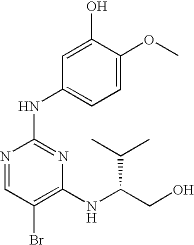 |
| Ex.No. | 170 * | 171 | 172 | 173 |
| Yield | 64% | 7% | 65% | 40% |
| Melting | 226° C. | 164° C. | 206° C. | 144° C. |
| Point | | | | |
| Mass | 525 (ES) | 488 (ES) | 395 (ES) | 397 (ES) |
| | 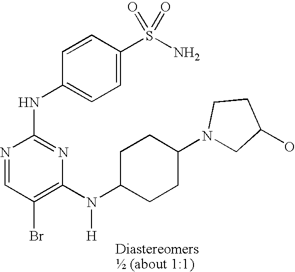 | 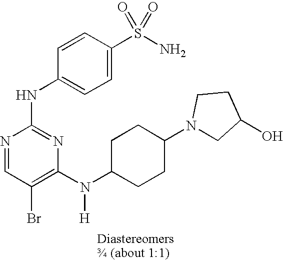 | 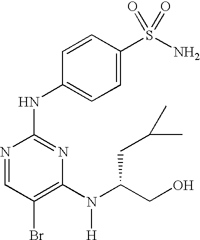 | 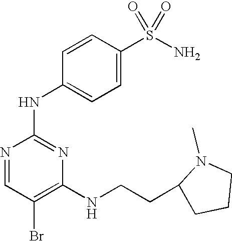 |
| Ex.No. | 174 * | 175 * | 176 | 177 |
| Yield | 95% | 51% | 3% | 8% |
| Melting | | | | |
| Point | | | | |
| Mass | 511 (ES) | 511 (ES) | 443 (EI) | 456 (EI) |
| | 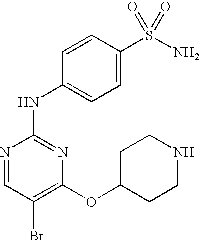 | 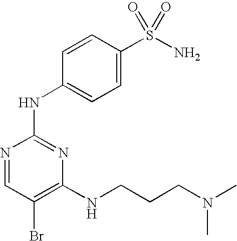 | 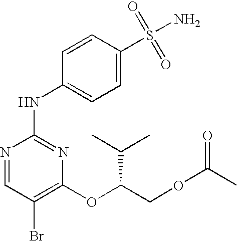 | 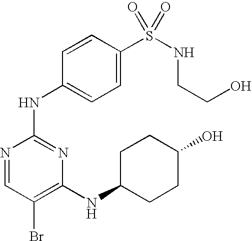 |
| Ex.No. | 178 | 179 | 180 | 181 |
| Yield | 17% | 9% | 27% | 24% |
| Melting | | | | |
| Point | | | | |
| Mass | 427 (EI) | 428 (ED | 472 (ES) | 486 (ES) |
| | 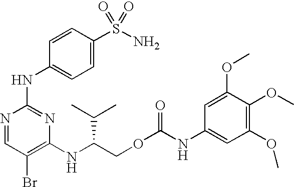 | 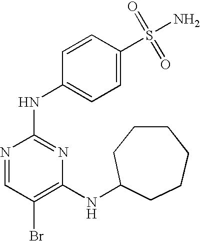 | 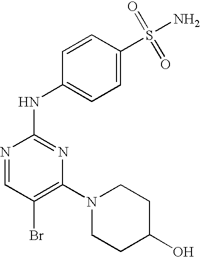 | 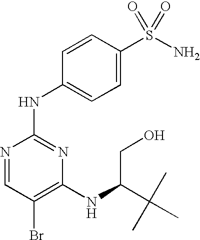 |
| Ex.No. | 182 | 183 | 184 | 185 |
| Yield | 57% | 78% | 26% | 76% |
| Melting | | | | |
| Point | | | | |
| Mass | 639 (ES) | 439 (EI) | 348 (EI) | 445 (EI) |
| | 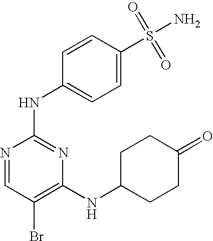 | 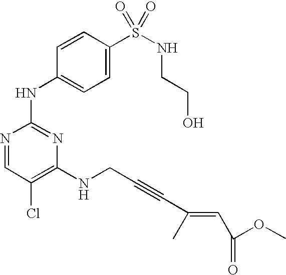 | 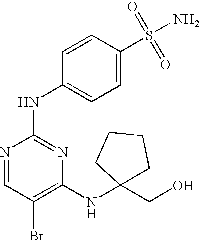 | 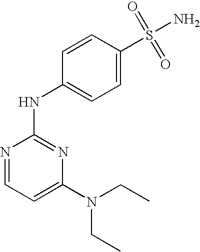 |
| Ex.No. | 186 | 187 | 188 | 189 |
| Yield | 16% | 7% | 61% | 35% |
| Melting | | | | |
| Point | | | | |
| Mass | 440 (ES) | 480 (ES) | 443 (EI) | 321 (EI) |
| | 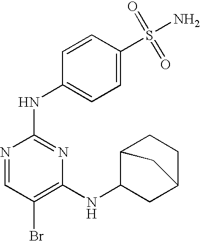 | 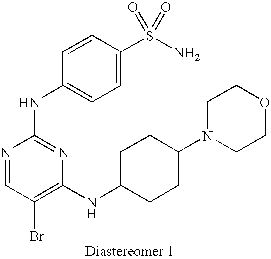 | 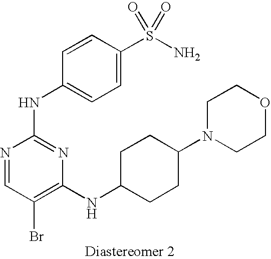 | 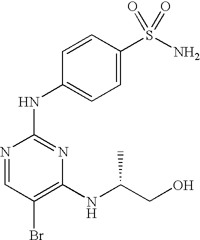 |
| Ex.No. | 190 | 191 * | 192 * | 193 |
| Yield | 63% | 15% | 17% | 57% |
| Melting | | | | |
| Point | | | | |
| Mass | 437 (EI) | 511 (ES) | 511 (EI) | 403 (EI) |
| | 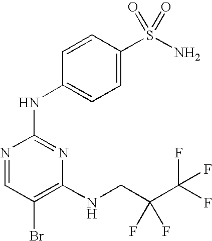 | 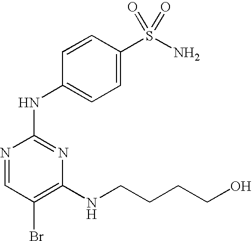 | 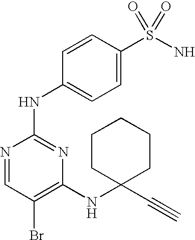 | 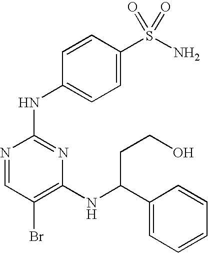 |
| Ex.No. | 194 | 195 | 196 | 197 |
| Yield | 26% | 56% | 12% | 61% |
| Melting | | | | |
| Point | | | | |
| Mass | 476 (EI) | 417 (EI) | 450 (EI) | 479 (EI) |
| | 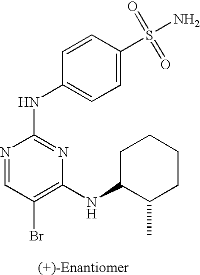 | 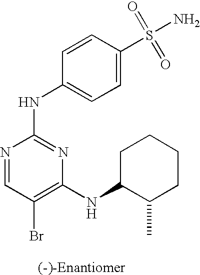 | 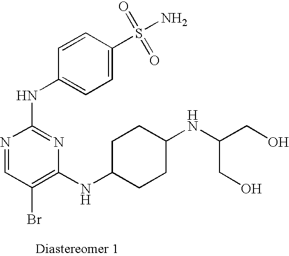 | 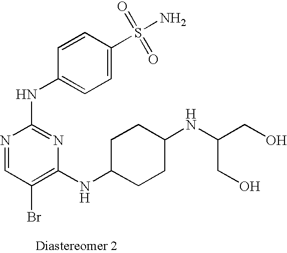 |
| Ex.No. | 198 | 199 | 200 | 201 |
| Yield | 4% | 4% | 7% | 2% |
| Melting | | | | |
| Point | | | | |
| Mass | 439 (EI) | 439 (EI) | 515 (ES) | 515 (ES) |
| | 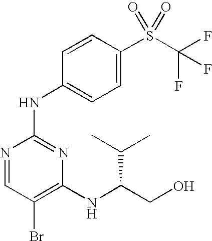 | 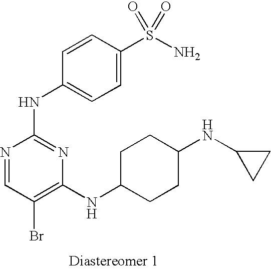 | 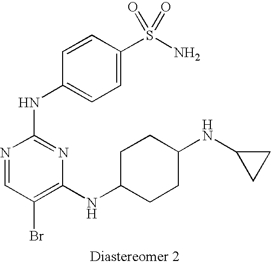 | 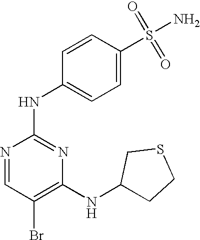 |
| Ex. No. | 202 | 203 * | 204 * | 205 |
| Yield | 10% | 2% | 2% | 16% |
| Melting | | | | |
| Point | | | | |
| Mass | 483 (ES) | 480 (EI) | 480 (EI) | 430 (ES) |
| | 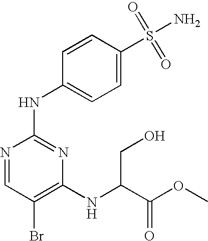 | 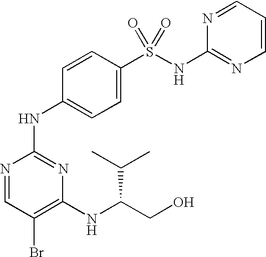 | 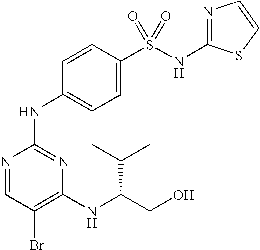 | 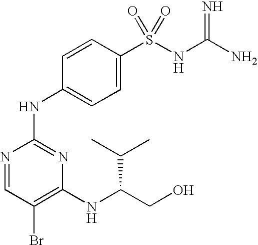 |
| Ex.No. | 206 | 207 | 208 | 209 |
| Yield | 5% | 55% | 44% | 77% |
| Melting | 223° C. | 248° C. | 228° C. | 231° C. |
| Point | | | | |
| Mass | 446 (ES) | 507 (EI) | 514 (EI) | |
| | 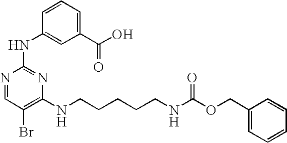 | 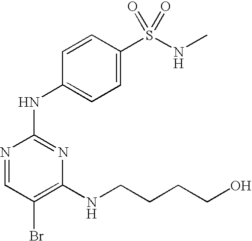 | 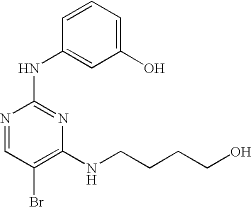 | 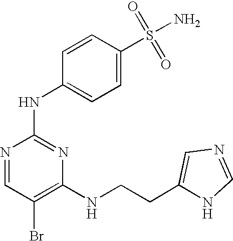 |
| Ex.No. | 210 | 211 | 212 | 71 |
| Yield | 86% | 22% | 41% | 77% |
| Melting | | | | |
| Point | | | | |
| Mass | 528 (CI) | 429 (EI) | 352 (EI) | 437 (EI) |
| | 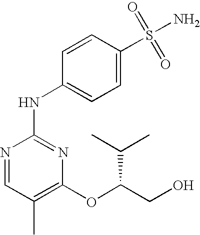 | 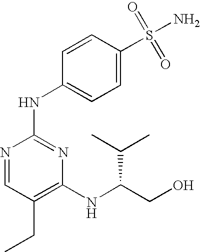 | 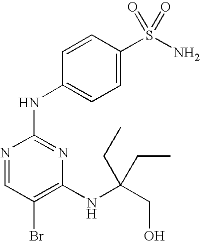 | 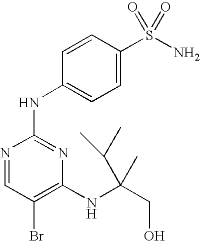 |
| Ex.No. | 213 | 61 | 214 | 215 |
| Yield | 49% | 25% | 2% | 9% |
| Melting | | | | |
| Point | | | | |
| Mass | 365 (EI) | 379 (EI) | 443 (ES) | 444 (ES) |
| | 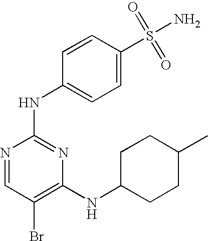 | 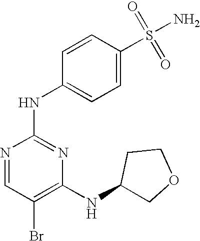 | 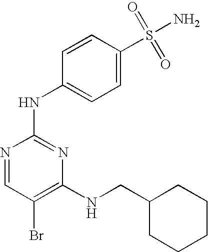 | 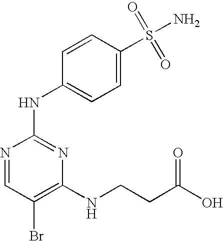 |
| Ex.No. | 216 | 217 | 218 | 219 |
| Yield | 65% | 34% | 58% | 88% |
| Melting | 239° C. | 239° C. | 238° C. | 280° C. |
| Point | | | | |
| Mass | 439(EI) | 413(EI) | 439(EI) | 416(EI) |
| | 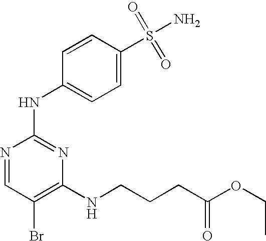 | 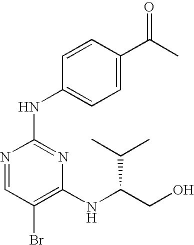 | 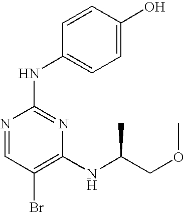 | 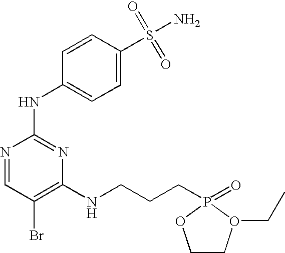 |
| Ex.No. | 74 | 56 | 220 | 221 |
| Yield | 7% | 17% | 65% | 19% |
| Melting | 285° C. | 158° C. | 166° C. | |
| Point | | | | |
| Mass | 457 (EI) | 392 (EI) | 354 (EI) | 522 (ES) |
| | 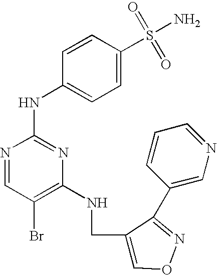 | 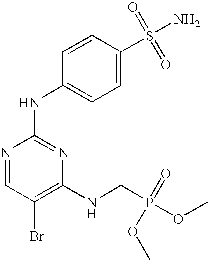 | 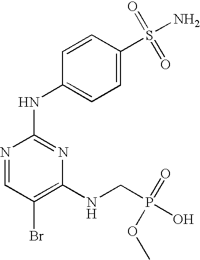 | 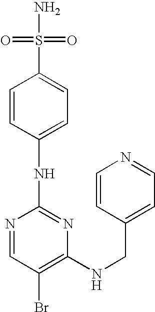 |
| Ex.No. | 222 | 223 | 224 | 225 |
| Yield | 54% | 23% | 7% | 43 |
| Melting | 300° C. | 300° C. | | 243° C. |
| Point | | | | |
| Mass | 501 (EI) | 465 (EI) | | 434 (EI) |
| | 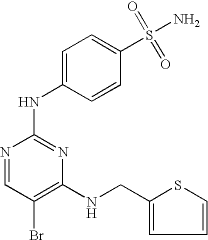 | 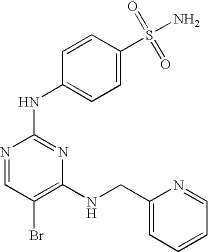 | 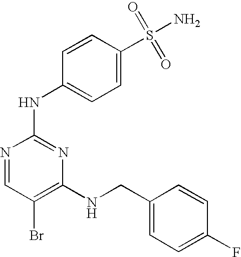 | 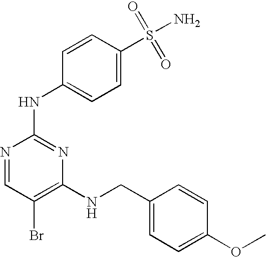 |
| Ex.No. | 226 | 227 | 228 | 229 |
| Yield | 47% | 41% | 88% | 89% |
| Melting | 229° C. | 287° C. | 259° C. | 233° C. |
| Point | | | | |
| Mass | 440 (CI) | 434 (EI) | 451 (EI) | 463 (EI) |
| | 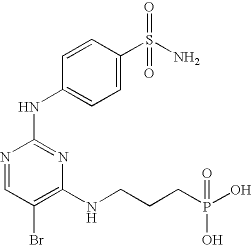 | | | |
| Ex. No. | 230 | | | |
| Yield | 58% | | | |
| Melting | >300° C. | | | |
| Point | | | | |
| Mass | 466 (ES) | | | |
| | 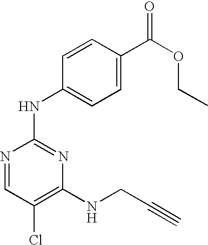 | 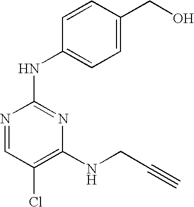 | 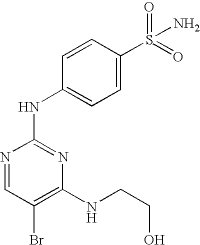 | 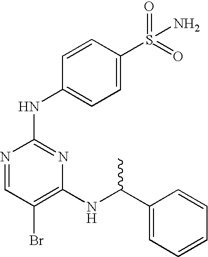 |
| Ex.No. | 231 | 232 | 233 | 234 |
| Yield | 85% | 35% | 33% | 25% |
| Melting | | | | |
| Point | | | | |
| Mass | 330 (EI) | 288 (EI) | 389 (CI) | 448 (ESI) |
| | 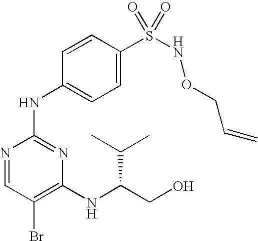 | 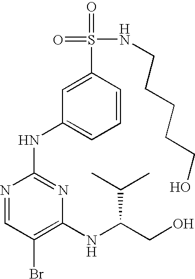 | 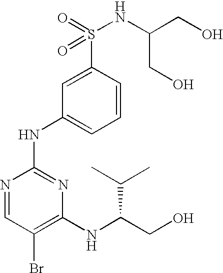 | 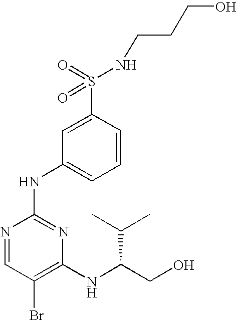 |
| Ex.No. | 235 | 236 | 237 | 238 |
| Melting | | | | |
| Point | | | | |
| Mass | 486 (ES) | 516 (ES) | 504 (ES) | 488 (ES) |
| | 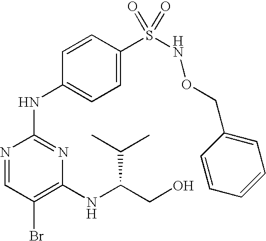 | 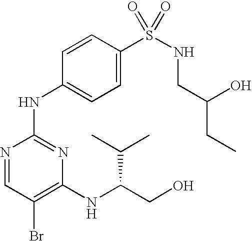 | 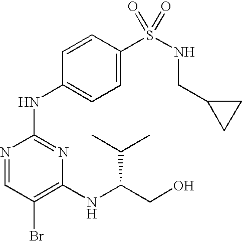 | 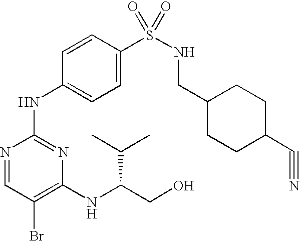 |
| Ex.No. | 239 | 240 | 241 | 242 |
| Melting | | | | |
| Point | | | | |
| Mass | 536 (ES) | 502 (ES) | 484 (ES) | 551 (ES) |
| | 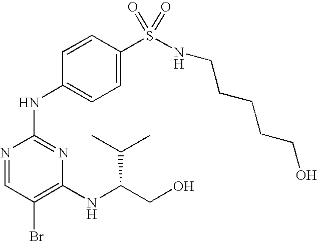 | 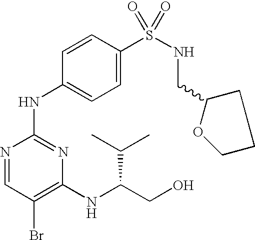 | 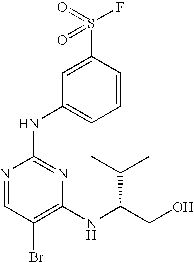 | |
| Ex.No. | 243 | 244 | 245 | |
| Melting | | | | |
| Point | | | | |
| Mass | 516 (ES) | 514 (ES) | 433 (ES) | |
| | 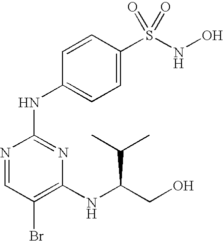 | 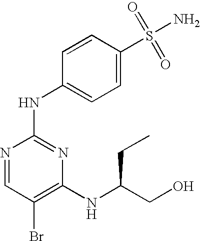 | 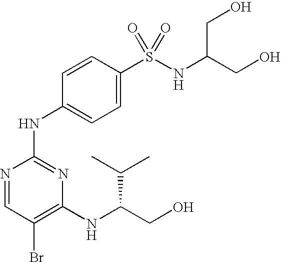 | 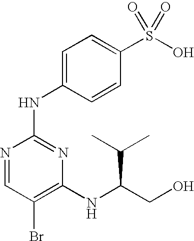 |
| Ex.No. | 246 | 247. | 248 | 249 |
| Melting | | | 205° C. | >300° C. |
| Point | | | | |
| Mass | 446 (ES) | 415 (EI) | 504 (ES) | 431 (ES) |
| | 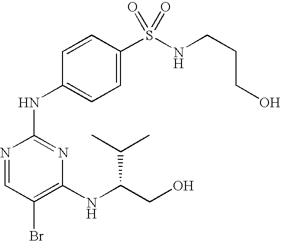 | 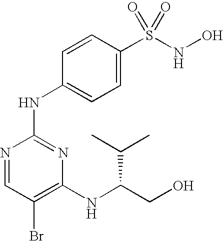 | 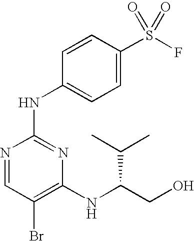 | 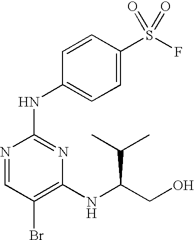 |
| Ex.No. | 250 | 251 | 252 | 253 |
| Melting | 113° C. | 231° C. | 187° C. | |
| Point | | | | |
| Mass | 488 (ES) | 446 (ES) | 433 (ES) | |
| | 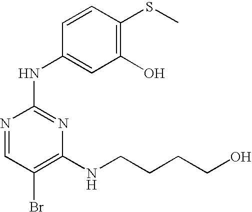 | 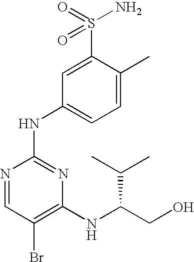 | 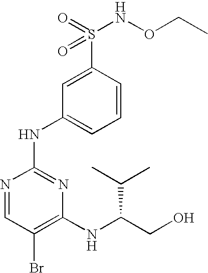 | 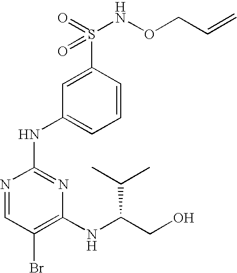 |
| Ex.No. | 254 | 255 | 256 | 257 |
| Melting | | | | |
| Point | | | | |
| Mass | 399 (ES) | 444 (ES) | 474 (ES) | 486 (ES) |

Compounds Nos. 159, 160, 161, 163, 167, 168, 170, 174, 175, 191, 192, 203 and 204 that are identified by * ) can be produced by the process variant described under Example No. 295.

### EXAMPLE 258

### Production of 4-(5-Bromo-4-morpholin-4-yl-pyrimidin-2-ylamino)-phenylsulfonamide

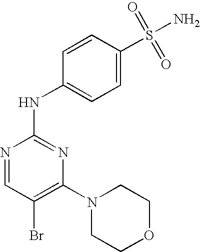

202 mg (0.60 mmol) of the compound of Example No. 122 is mixed with 1 ml of water as well as 0.2 g (1.2 mmol) of bromine and stirred at room temperature. After 24-hours, 0.2 g (1.2 mmol) of bromine is added again and stirred for another 24 hours at room temperature. The solvent is evaporated by means of underpressure, and the remaining residue is purified by chromatography (Flashmaster II, DCM/MeOH 7:3). The yield is 17 ing (0.04 mmol, 7%) of the product as a white solid.

| | 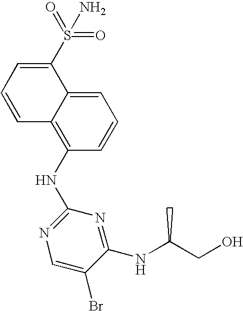 | 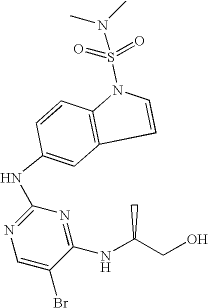 | 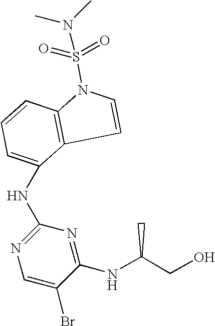 | 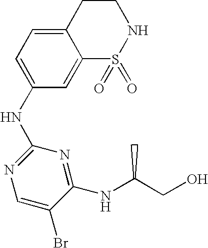 |
|---|---|---|---|---|
| Ex.No. | 259 | 260 | 261 | 262 |
| Melting | | 205-207° C. | 202-203° C. | |
| Point | | | | |
| Mass | MS (ES) 452, 454 | | | 428 (ES) |
| | (M + H, 100%) | | | |

| | 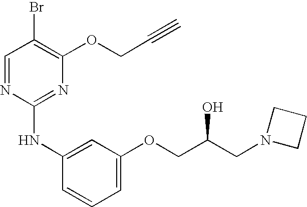 | 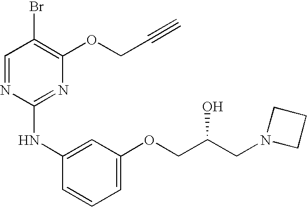 | 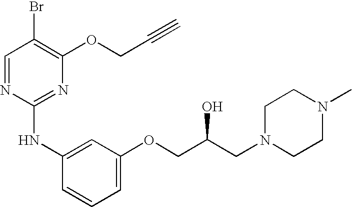 |
|---|---|---|---|
| Ex. No. | 263 | 264 | 265 |
| ESI-MS | 434 | 434 | 477 |
| | 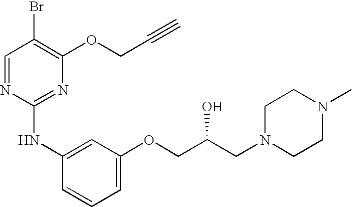 | 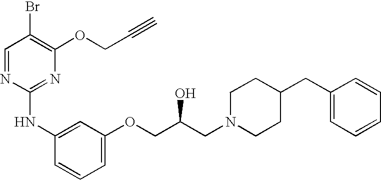 | 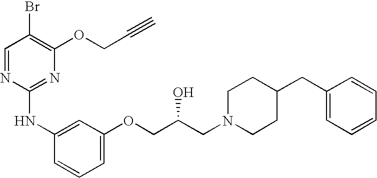 |
| Ex. No. | 266 | 267 | 268 |
| ESI-MS | 477 | 552 | 552 |

Analogously to production example 1, the following compounds were also produced:

| | 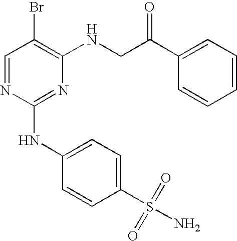 | 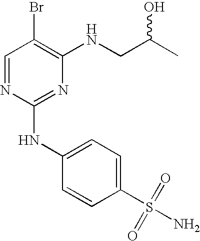 | 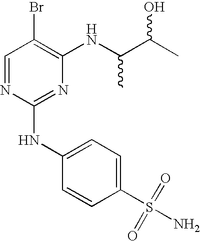 | 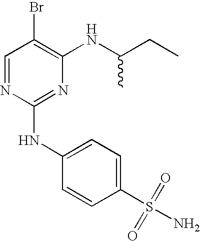 |
|---|---|---|---|---|
| Ex.No. | 272 | 273 | 274 | 275 |
| Yield | 61% | 44% | 42% | 68% |
| Mass | EI: | EI: | ESI: | EI: |
| | M⁺ 463 (4%) | M⁺ 403 (24%) | MH⁺ 418 100% | M⁺ 401 (33%) |
| | 277 (8%) | 358 (100%) | 416 (94%) | 372 (100%) |
| | 105 (100%) | 277 (52%) | 346 (8%) | 344 (38%) |
| | 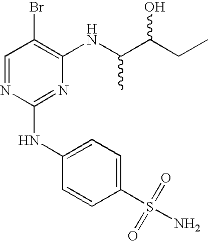 | 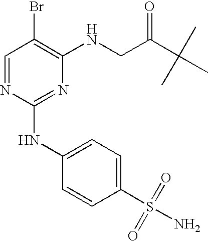 | 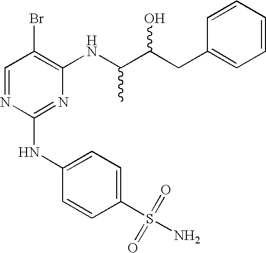 | 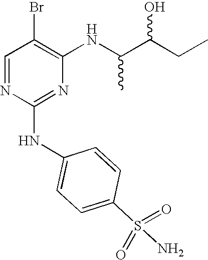 |
| Ex. No. | 276 | 277 | 278 | 279 |
| Yield | 81% | 58% | 20% | 30% |
| Mass | EI: | ESI: | ESI: | ESI: |
| | M⁺ 431 | MH⁺ 444 | MH⁺ 494 (75%) | MH⁺ 418 |
| | (5%) | (100%) | 346 (18%) | (100%) |
| | 372 (100%) 442 (97%) | 214 (55%) | 416 (97%) | |
| | 291 (46%) | 115 (20%) | | 310 (27%) |
| | 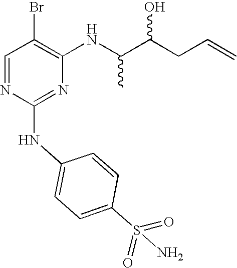 | 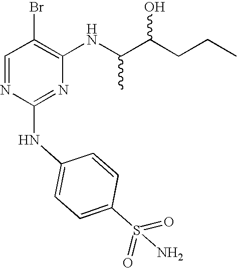 | 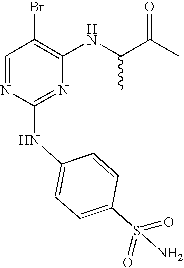 | 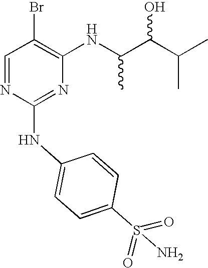 |
| Ex.No. | 280 | 281 | 282 | 283 |
| Yield | 55% | 43% | ˜18% | 35% |
| Mass | ESI: | ESI: | ESI: | ESI: |
| | MH⁺ 444 | MH⁺ 446 | MH⁺ 416 | MH⁺ 446 |
| | (100%) | (100%) | (100%) | (100%) |
| | 442 (97%) | 444 (95%) | 414 (96%) | 444 (90%) |
| | 214 (12%) | 346 (5%) | 317 (4%) | |
| | 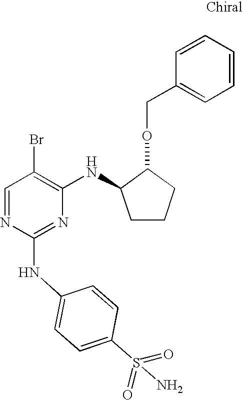 | 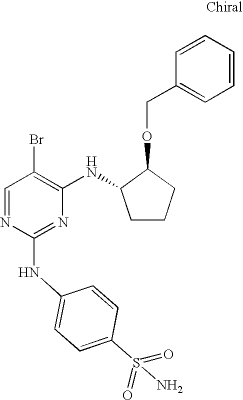 | 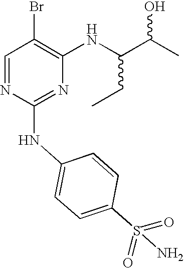 | 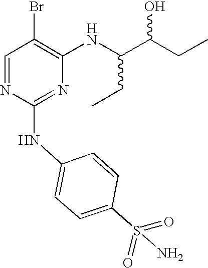 |
| Ex.No. | 284 | 285 | 286 | 287 |
| Yield | 51% | 46% | 47% | 61% |
| Mass | ESI: | ESI: | ESI. | ESI. |
| | MH⁺ 520 | MH⁺ 520 | MH⁺ 432 | MIH⁺ 446 |
| | (100%) | (100%) | (100%) | (100%) |
| | 518 (97%) | 518 (97%) | 430 (95%) | 444 (93%) |
| | 115 (27%) | 115 (23%) | 346 (5%) | 115 (13%) |

According to subsequent production variants, the following compounds are also synthesized:

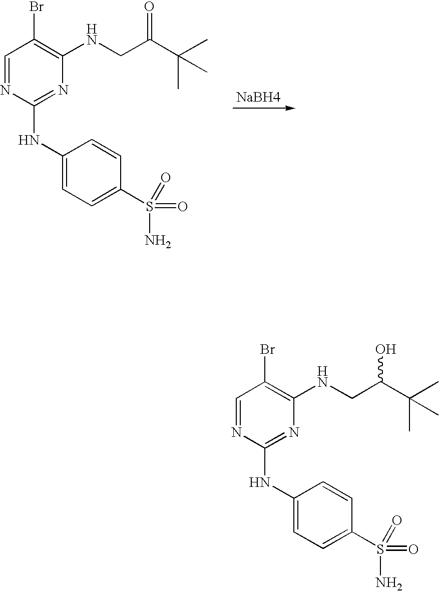

30 mg (0.0678 mmol) of compound No. 277 is dissolved in 1 ml of methanol/tetrahydrofuran 1:1. After 10 mg of sodium borohydride is added, it is stirred for 2 more hours. Then, it is quenched with 3-4 drops of glacial acetic acid while being cooled and concentrated by evaporation. The crude product is subsequently taken up with a little water, suctioned off, rewashed with acetonitrile and dried at 60° C. in a vacuum. Yield: 21 mg (70% of theory) of the desired compound.

| | 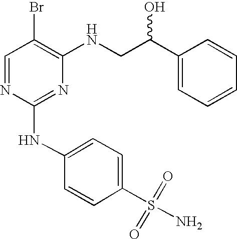 | 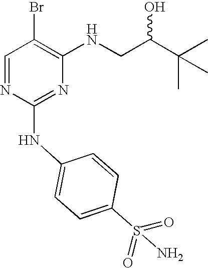 |
|---|---|---|
| Ex. No. | 288 | 289 |
| Yield | 52% | 70% |
| Mass | EI: | ESI: |
| | M⁺ 465 (5%) | MH⁺ 446 (100%) |
| | 358 (40%) | 444 (93%) |
| | 207 (31%) | 117 (20%) |

### EXAMPLE 290

### Production of the Oxime Ether-Pyrimidine Compounds of General Formula I

The production of oxime ether is carried out according to the following general reaction diagram: 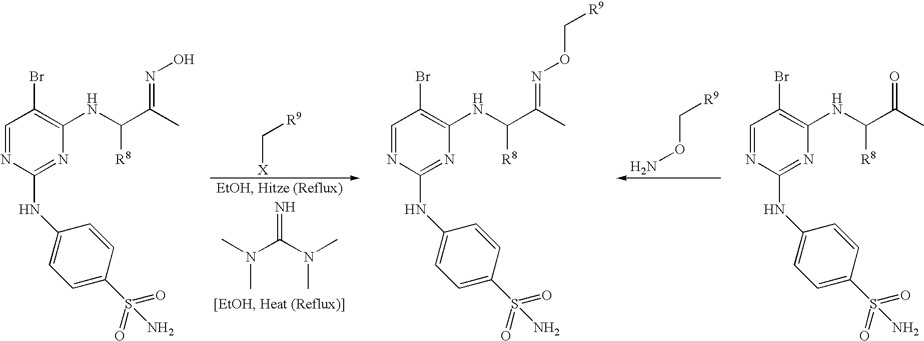
R⁸ and R⁹ have the meanings that are indicated in general formula I.

### PRODUCTION OF EXAMPLE 290

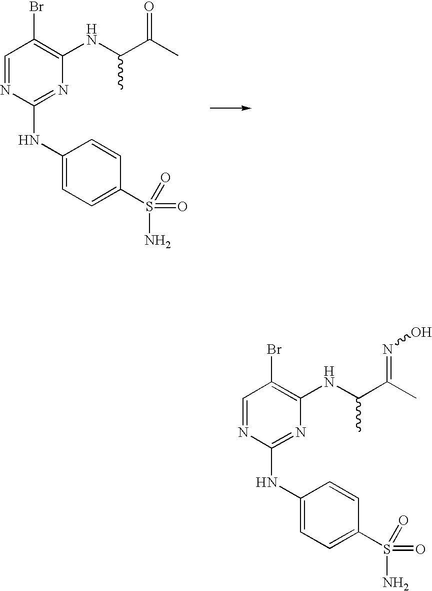

50 mg (0.12 mmol) of compound No. 282, 34 mg of hydroxylammonium chloride and 150 mg of pulverized KOH are refluxed for 2 hours in 2 ml of ethanol. Then, it is poured into ice water and acidified with glacial acetic acid, extracted 3 times with dichloromethane/isopropanol 4:1, dried and concentrated by evaporation with magnesium sulfate. The residue is suspended with acetonitrile, suctioned off and dried at 60° C. Yield: 28 mg (54% of theory) of the desired compound.

Mass: ESI:

MH⁺ 429 (29%)

371 (61%)

289 (91%)

The following compounds were also produced analogously:

| | 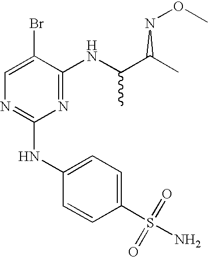 | 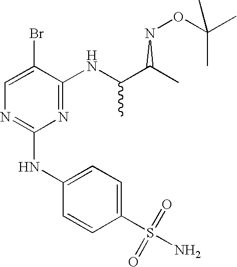 | 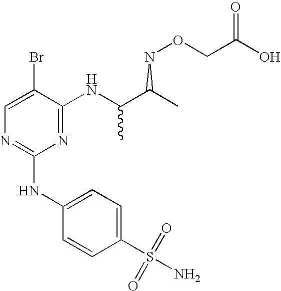 |
|---|---|---|---|
| Ex.No. | 291 | 292 | 293 |
| Yield | 34% | 36% | 40% |
| Mass | ESI: | ESI: | ESI: |
| | MH⁺ 443 (95%) | MH⁺ 485 (92%) | MH⁺ 487 (91%) |
| | 445 (99%) | 487 (99%) | 489 (89%) |
| | 373 (32%) | | 373 (32%) |

### EXAMPLE 294

Reductive Amination

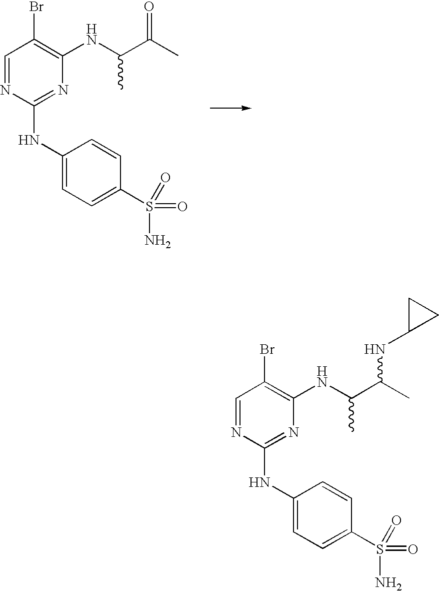

50 mg (0.12 mmol) of compound No. 282 and 7.5 mg (0.132 mmol) of cyclopropylamine are dissolved in 2 ml of 1,2-dichloroethane. After 9.1 mg (0.144 mmol) of sodium cyanoborohydride is added, it is allowed to stir for 12 more hours. Then, it is diluted with dichloromethane/isopropanol 4:1, washed twice with water, dried with magnesium sulfate and then concentrated by evaporation. The residue is chromatographed on silica gel with dichloromethane/methanol 95:5. Yield: 18 mg (33% of theory) of the desired compound.

| | 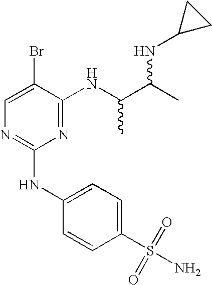 |
|---|---|
| Yield | 33% |
| Mass | ESI: |
| | MH⁺ 457 |
| | (98%) |
| | 455 (93%) |
| | 249 (55%) |

Similarly produced are also compounds Nos. 159, 160, 161, 163, 167, 168, 170, 174, 175, 191, 192, 203 and 204.

### EXAMPLES 295 AND 296

Produced in a way similar to Example 1 are also the following two compounds:

| | 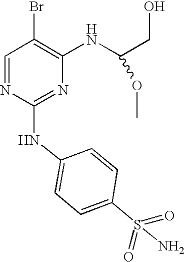 | 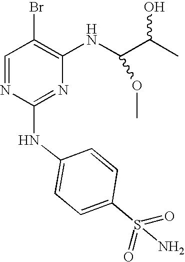 |
|---|---|---|
| Example | 295 | 296 |
| Yield | 46% | 47% |
| Mass | ESI: | ESI: |
| | 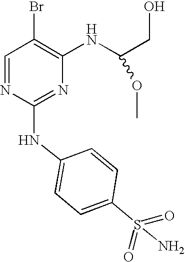 | 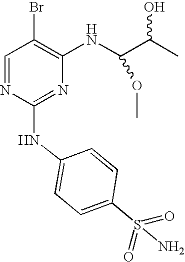 |
| | MH⁺ 432 (30%) | MH⁺ 446 (45%) |
| | 434 (31%) | 448 (49%) |
| | 123 (100%) | 123 (90%) |

### Production of the Sulfonamides of General Formula I

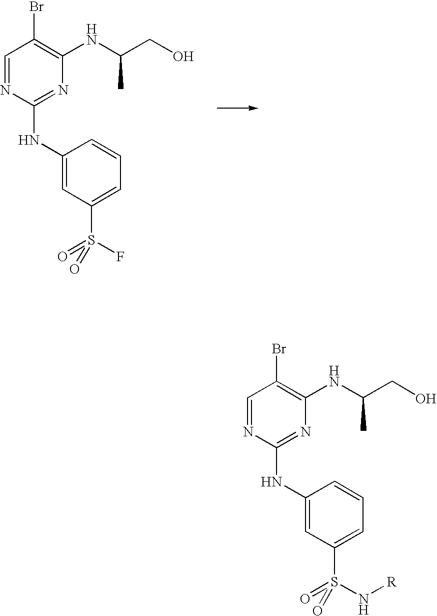

0.2 mmol of sulfonic acid fluoride is introduced into a reactor of a synthesizer, and 1.0 ml of solvent, preferably 2-butanol, is added. 0.2 ml (0.2 mmol) of DMAP—dissolved in a solvent, for example DMSO or 2-butanol—and 0.2 ml (0.2 mmol) of amine, dissolved in 2-butanol, are added in succession via a pipette. The reaction mixture is then stirred for 20 hours at 80° C. After the reaction is completed, the crude product is pipetted off, and the reactor is rewashed with 1.0 ml of THF. The solution of the crude product is then concentrated by evaporation and purified by means of HPLC.

The following compounds were produced:

| | 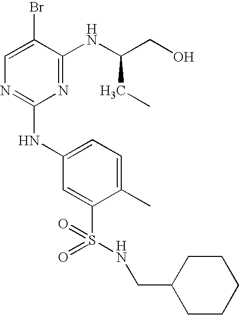 | 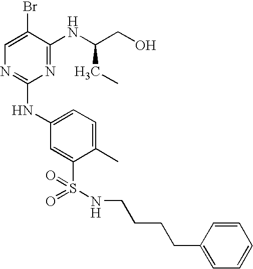 | 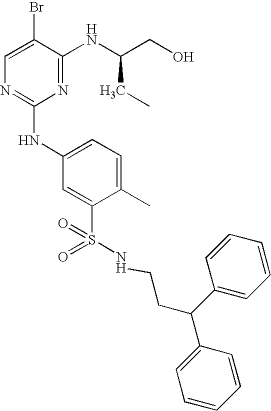 | 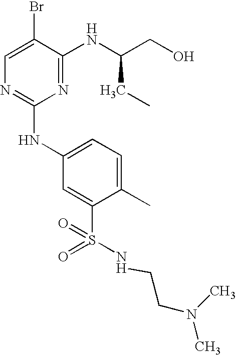 |
|---|---|---|---|---|
| Ex.No. | 297 | 298 | 299 | 300 |
| Molecular | 526.4968 | 562.5298 | 624.6006 | 501.4471 |
| Weight | | | | |
| ESI-MS | 526/528 | 562/564 | 624/626 | 501/503 |
| | 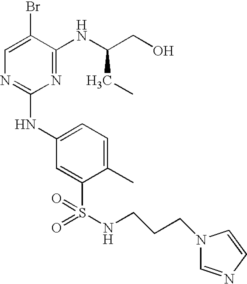 | 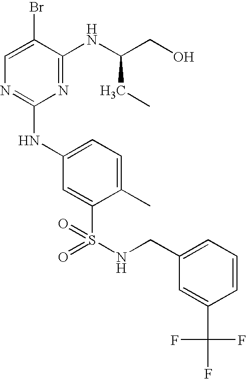 | 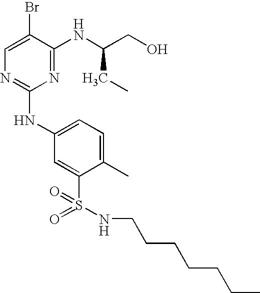 | 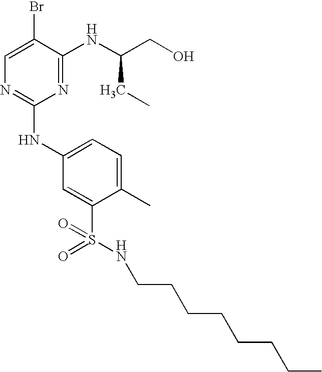 |
| Ex. No. | 301 | 302 | 303 | 304 |
| Molecular | 538.4682 | 588.4465 | 528.5126 | 542.5394 |
| Weight | | | | |
| ESI-MS | 538/540 | 588/590 | 528/530 | 542/544 |
| | 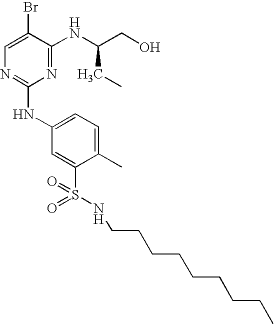 | 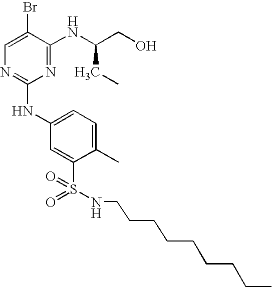 | 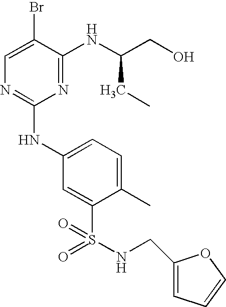 | 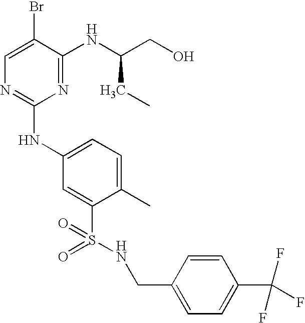 |
| Ex.No. | 305 | 306 | 307 | 308 |
| Molecular | 556.5662 | 570.593 | 510.4106 | 588.4465 |
| Weight | | | | |
| ESI-MS | 556/558 | 570/572 | 510/512 | 588/590 |
| | 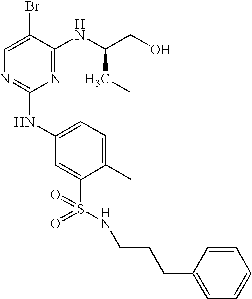 | 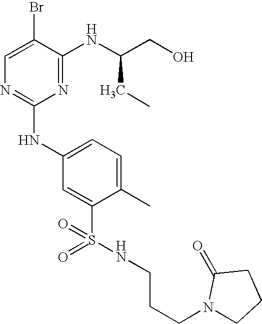 | 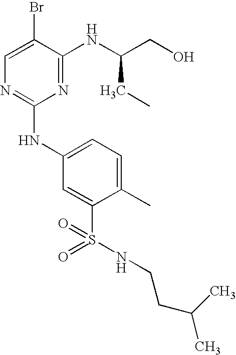 | 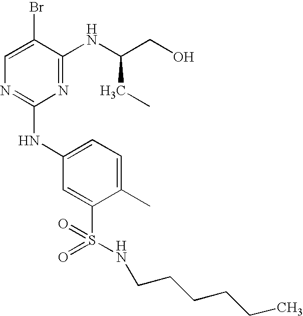 |
| Ex.No. | 309 | 310 | 311 | 312 |
| Molecular | 548.503 | 555.4949 | 500.459 | 514.4858 |
| Weight | | | | |
| ESI-MS | 548/550 | 555/557 | 500/502 | 514/516 |
| | 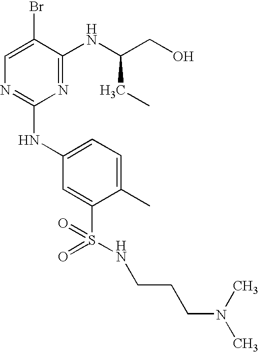 | 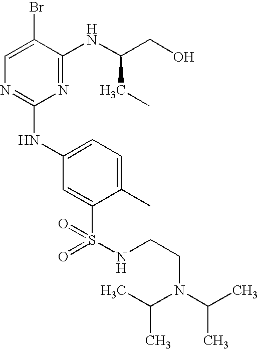 | 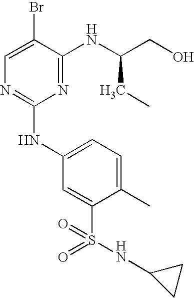 | 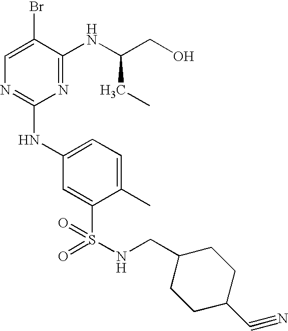 |
| Ex.No. | 313 | 314 | 315 | 316 |
| Molecular | 515.4739 | 557.5543 | 470.3896 | 551.5069 |
| Weight | | | | |
| ESI-MS | 515/517 | 557/559 | 470/472 | 55 1/553 |
| | 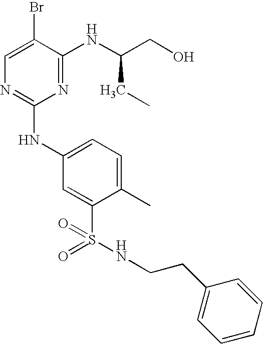 | 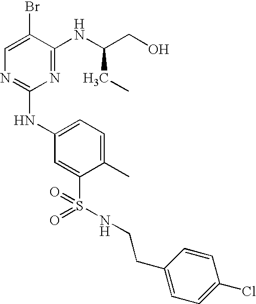 | 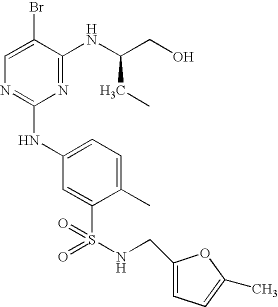 | 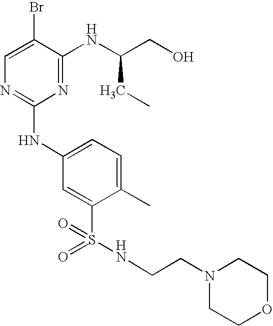 |
| Ex.No. | 317 | 318 | 319 | 320 |
| Molecular | 534.4762 | 568.9213 | 524.4374 | 543.4839 |
| Weight | | | | |
| ESI-MS | 534/536 | 568/570 | 524/526 | 543/545 |
| | 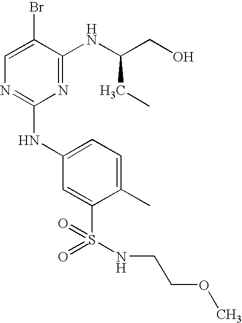 | 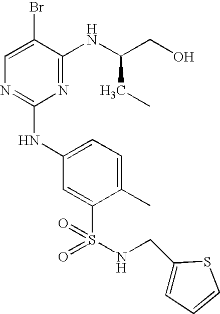 | 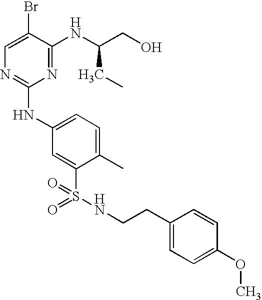 | 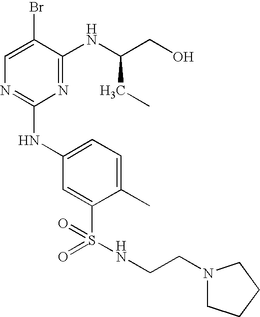 |
| Ex. No. | 321 | 322 | 323 | 324 |
| Molecular | 488.4044 | 526.4776 | 564.502 | 527.4849 |
| Weight | | | | |
| ESI-MS | 488/490 | 526/528 | 564/566 | 527/529 |
| | 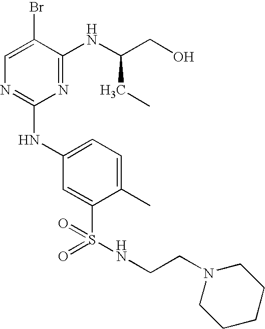 | 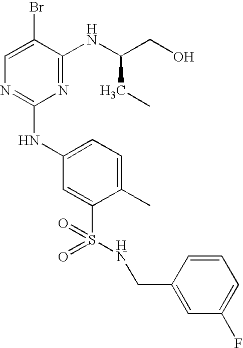 | 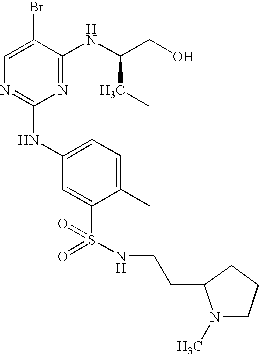 | 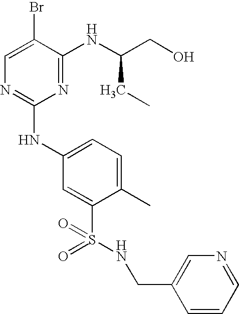 |
| Ex.No. | 325 | 326 | 327 | 328 |
| Molecular | 541.5117 | 538.4395 | 541.5117 | 521.4375 |
| Weight | | | | |
| ESI-MS | 541/543 | 538/540 | 541/543 | 521/523 |
| | 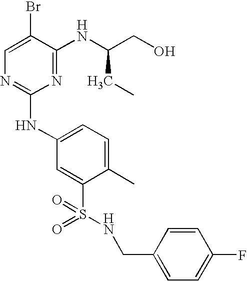 | 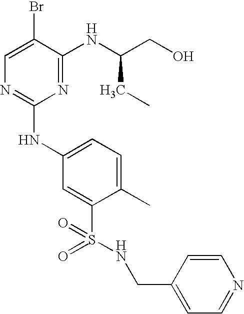 | 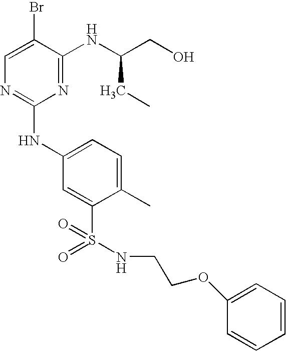 | 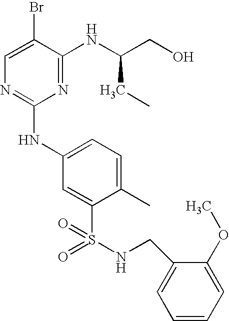 |
| Ex.No. | 329 | 330 | 331 | 332 |
| Molecular | 538.4395 | 521.4375 | 550.4752 | 550.4752 |
| Weight | | | | |
| ESI-MS | 538/540 | 521/523 | 550/552 | 550/552 |
| | 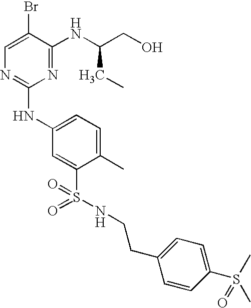 | 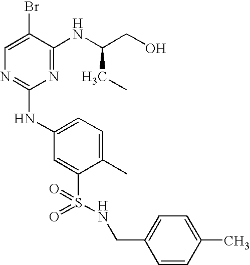 | 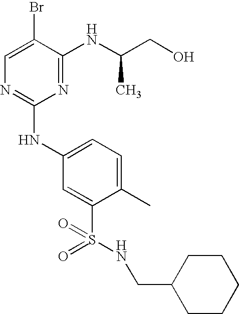 | 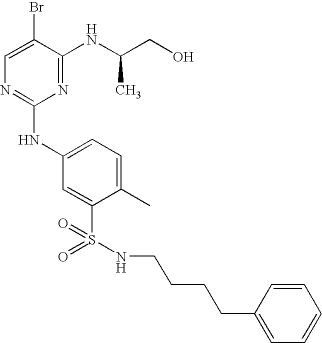 |
| Ex. No. | 333 | 334 | 335 | 336 |
| Molecular | 613.5551 | 534.4762 | 512.47 | 548.503 |
| Weight | | | | |
| ESI-MS | 613/615 | 534/536 | 512/514 | 548/550 |
| | 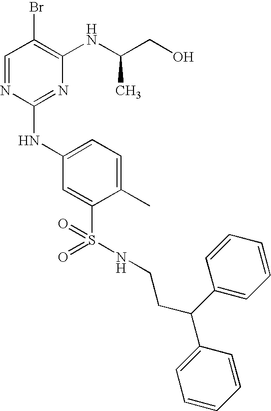 | 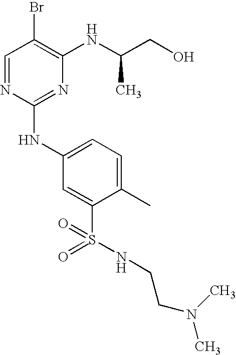 | 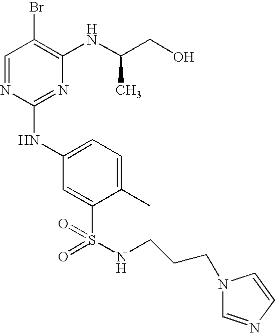 | 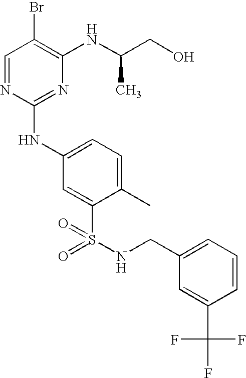 |
| Ex. No. | 337 | 338 | 339 | 340 |
| Molecular | 610.5738 | 487.4203 | 524.4414 | 574.4197 |
| Weight | | | | |
| ESI-MS | 610/612 | 487/489 | 524/526 | 574/576 |
| | 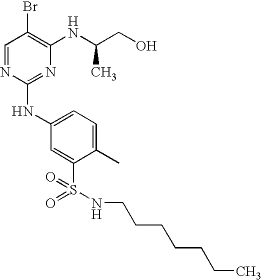 | 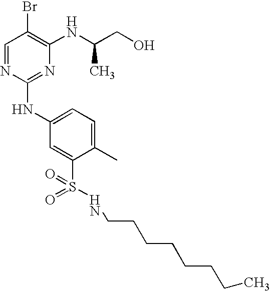 | 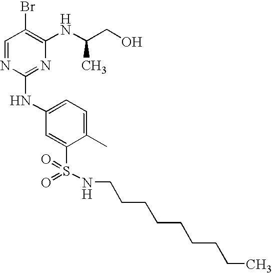 | 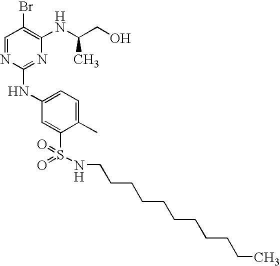 |
| Ex.No. | 341 | 342 | 343 | 344 |
| Molecular | 514.4858 | 528.5126 | 542.5394 | 556.5662 |
| Weight | | | | |
| ESI-MS | 516/514 | 528/530 | 542/544 | 556/558 |
| | 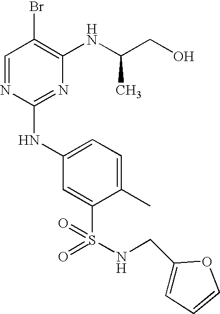 | 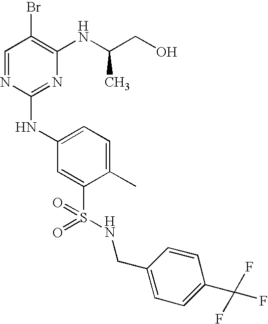 | 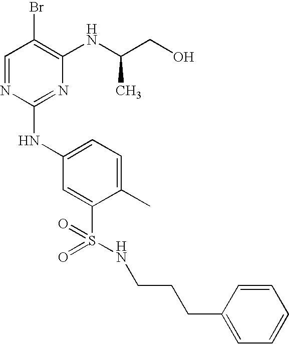 | 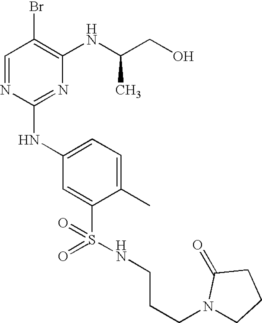 |
| Ex.No. | 345 | 346 | 347 | 348 |
| Molecular | 496.3838 | 574.4197 | 534.4762 | 541.4681 |
| Weight | | | | |
| ESI-MS | 496/498 | 574/576 | 534/536 | 541/543 |
| | 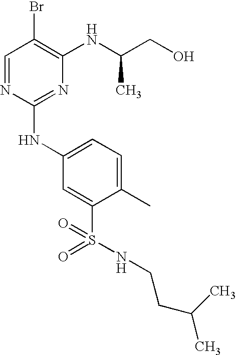 | 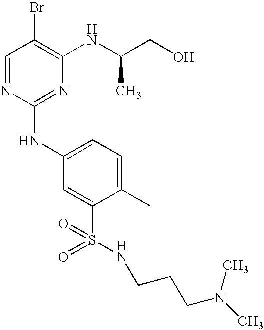 | 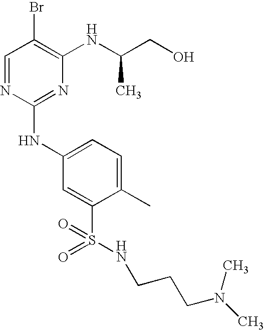 | 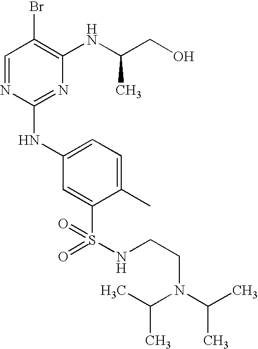 |
| Ex.No. | 349 | 350 | 351 | 352 |
| Molecular | 486.4322 | 500.459 | 501.4471 | 543.5275 |
| Weight | | | | |
| ESI-MS | 486/488 | 500/502 | 501/503 | 543/545 |
| | 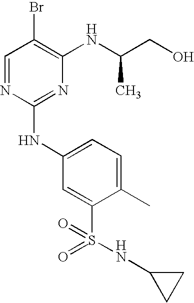 | 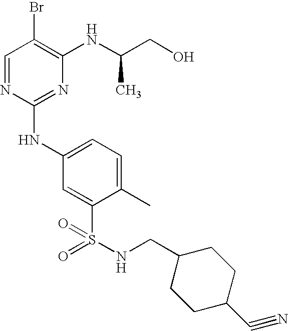 | 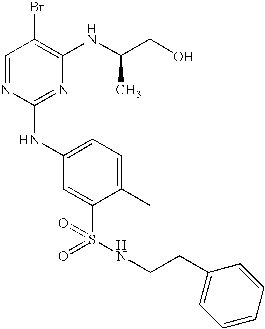 | 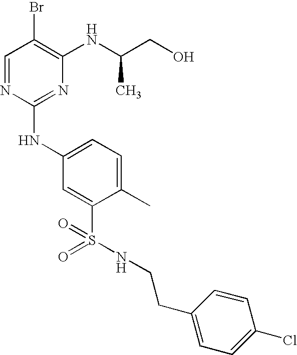 |
| Ex.No. | 353 | 354 | 355 | 356 |
| Molecular | 456.3628 | 537.4801 | 520.4494 | 554.8945 |
| Weight | | | | |
| ESI-MS | 456/458 | 537/539 | 520/522 | 554/556 |
| | 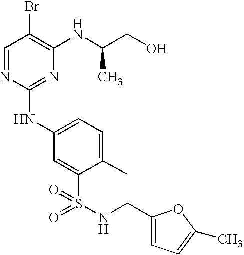 | 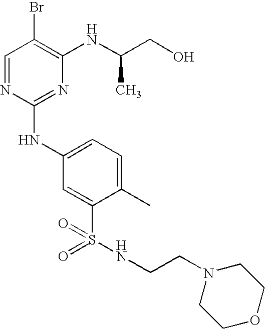 | 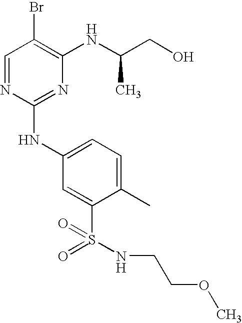 | 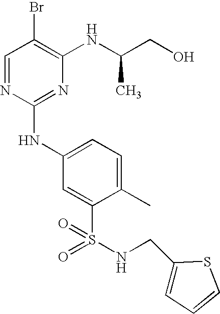 |
| Ex.No. | 357 | 358 | 359 | 360 |
| Molecular | 510.4106 | 529.4571 | 474.3776 | 512.4508 |
| Weight | | | | |
| ESI-MS | 510/512 | 529/531 | 474/476 | 541/514 |
| | 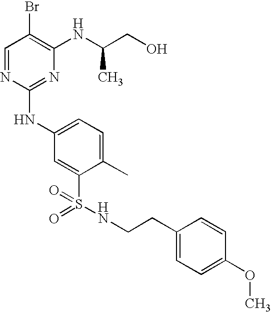 | 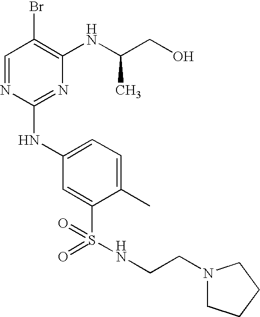 | 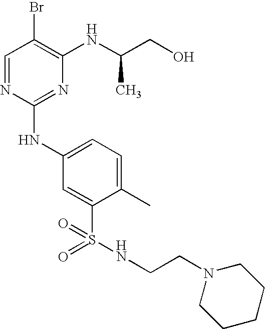 | 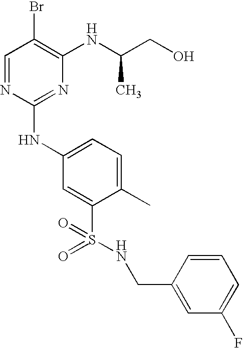 |
| Ex.No. | 361 | 362 | 363 | 364 |
| Molecular | 550.4752 | 513.4581 | 527.4849 | 524.4127 |
| Weight | | | | |
| ESI-MS | 550/552 | 513/515 | 527/529 | 524/526 |
| | 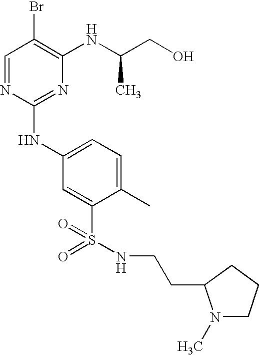 | 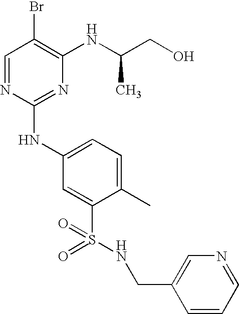 | 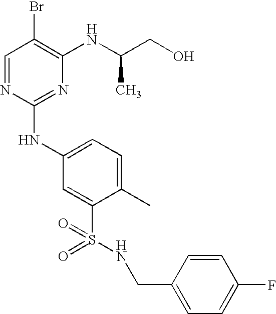 | 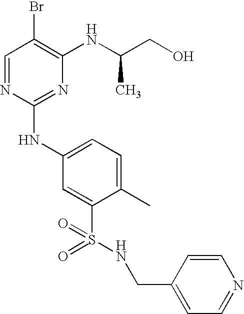 |
| Ex.No. | 365 | 366 | 367 | 368 |
| Molecular | 527.4849 | 507.4107 | 524.4127 | 507.4107 |
| Weight | | | | |
| ESI-MS | 527/529 | 507/509 | 524/526 | 507/509 |
| | 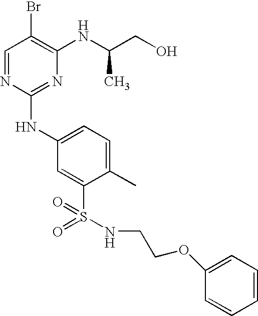 | 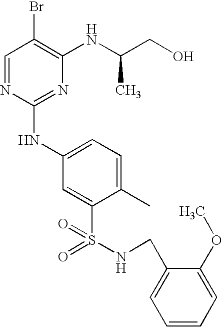 | 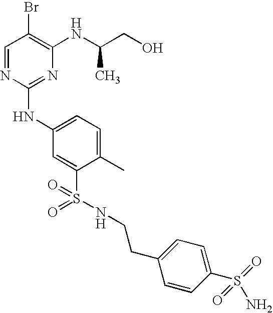 | 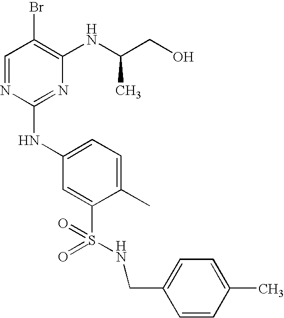 |
| Ex.No. | 369 | 370 | 371 | 372 |
| Molecular | 536.4484 | 536.4484 | 599.5283 | 520.4494 |
| Weight | | | | |
| ESI-MS | 536/538 | 536/538 | 599/601 | 520/522 |
| | 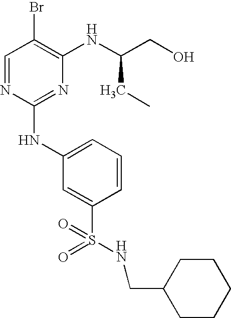 | 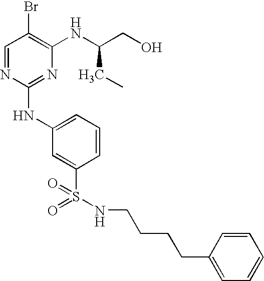 | 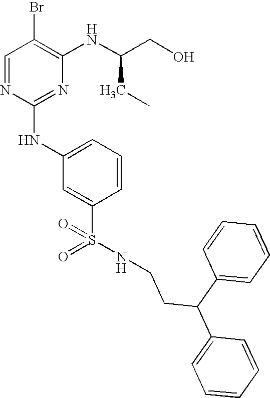 | 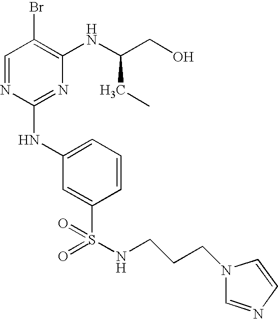 |
| Ex.No. | 373 | 374 | 375 | 376 |
| Molecular | 512.47 | 548.503 | 610.5738 | 524.4414 |
| Weight | | | | |
| ESI-MS | 512/514 | 548/550 | 610/612 | 524/526 |
| | 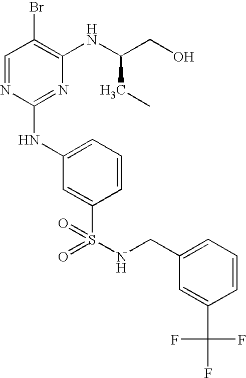 | 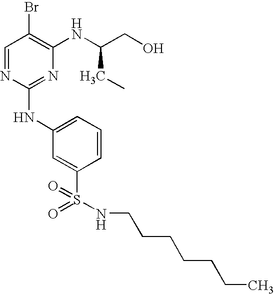 | 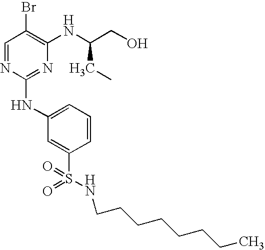 | 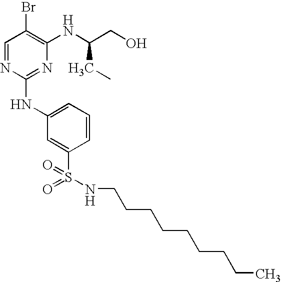 |
| Ex.No. | 377 | 378 | 379 | 380 |
| Molecular | 574.4197 | 514.4858 | 528.5126 | 542.5394 |
| Weight | | | | |
| ESI-MS | 574/576 | 514/516 | 528/530 | 542/544 |
| | 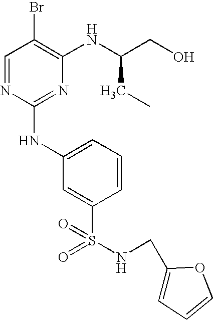 | 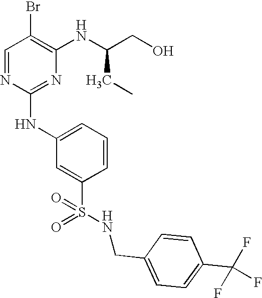 | 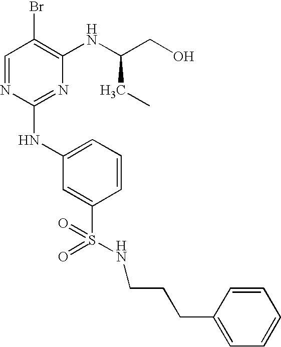 | 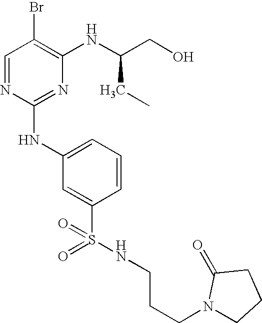 |
| Ex.No. | 381 | 382 | 383 | 384 |
| Molecular | 496.3838 | 574.4197 | 534.4762 | 541.4681 |
| Weight | | | | |
| ESI-MS | 496/498 | 574/576 | 534/536 | 541/543 |
| | 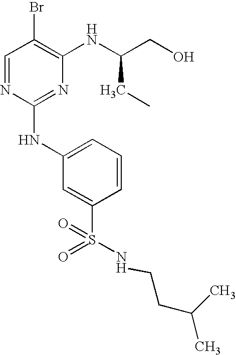 | 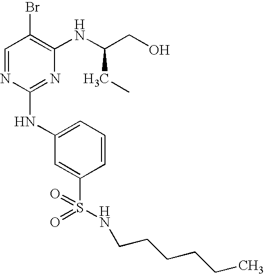 | 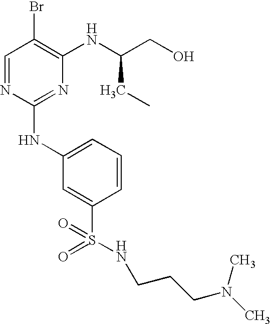 | 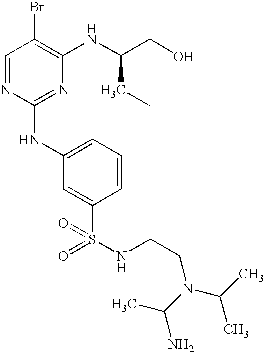 |
| Ex.No. | 385 | 386 | 387 | 388 |
| Molecular | 486.4322 | 500.459 | 501.4471 | 543.5275 |
| Weight | | | | |
| ESI-MS | 486/488 | 500/502 | 501/503 | 543/545 |
| | 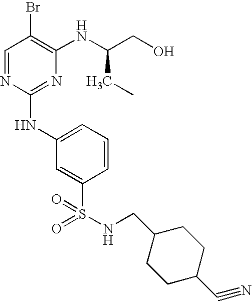 | 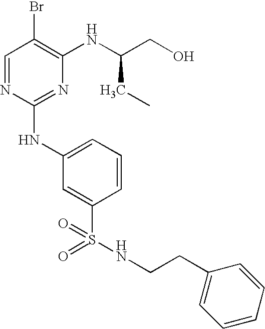 | 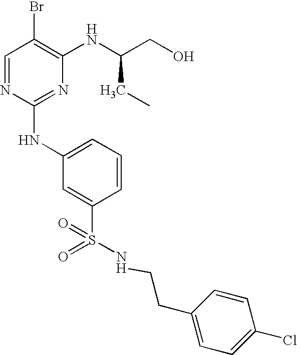 | 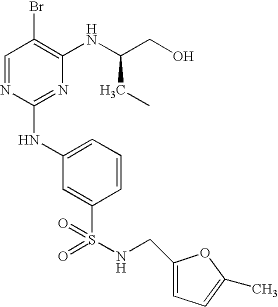 |
| Ex.No. | 389 | 390 | 391 | 392 |
| Molecular | 537.4801 | 520.4494 | 554.8945 | 510.4106 |
| Weight | | | | |
| ESI-MS | 537/539 | 520/522 | 554/556 | 510/512 |
| | 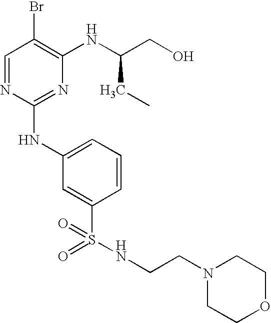 | 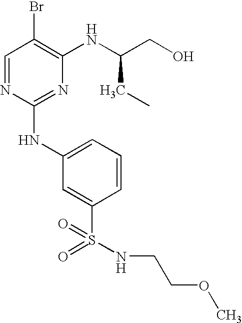 | 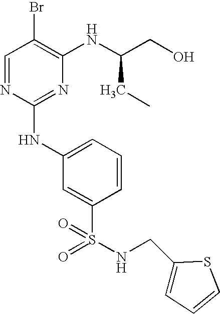 | 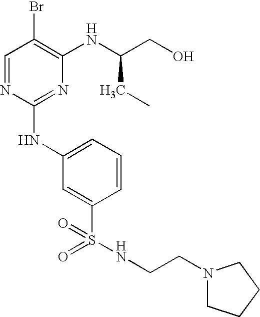 |
| Ex.No. | 393 | 394 | 395 | 396 |
| Molecular | 529.4571 | 474.3776 | 512.4508 | 513.4581 |
| Weight | | | | |
| ESI-MS | 529/531 | 474/476 | 512/514 | 513/515 |
| | 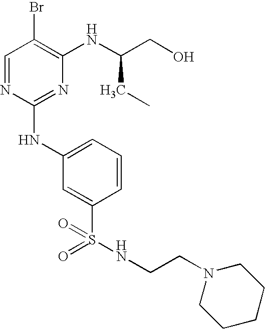 | 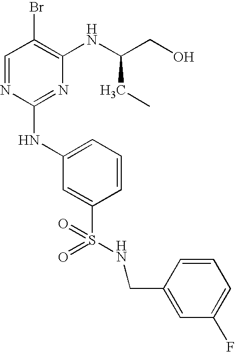 | 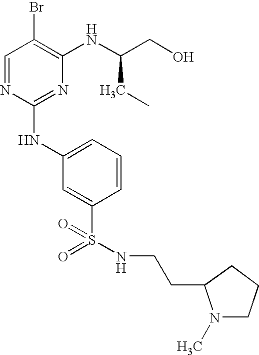 | 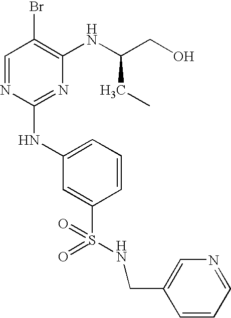 |
| Ex.No. | 397 | 398 | 399 | 400 |
| Molecular | 527.4849 | 524.4127 | 527.4849 | 507.4107 |
| Weight | | | | |
| ESI-MS | 527/529 | 524/526 | 527/529 | 507/509 |
| | 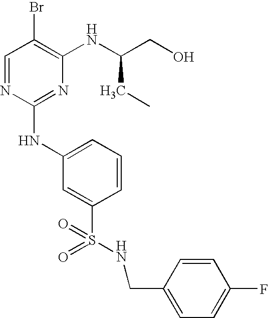 | 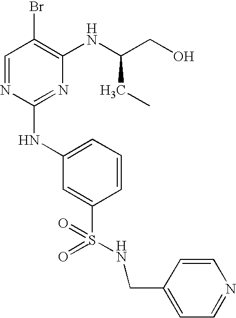 | 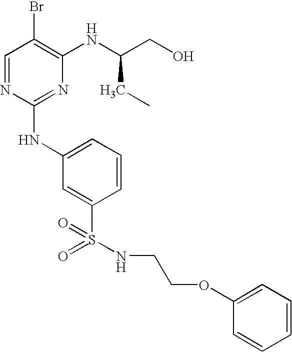 | 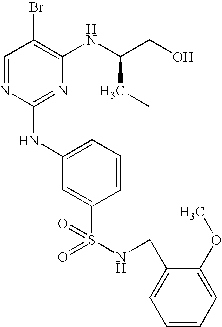 |
| Ex.No. | 401 | 402 | 403 | 404 |
| Molecular | 524.4127 | 507.4107 | 536.4484 | 536.4484 |
| Weight | | | | |
| ESI-MS | 524/526 | 507/509 | 526/538 | 536/538 |
| | 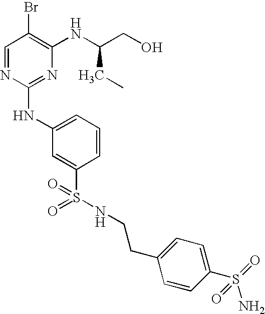 | 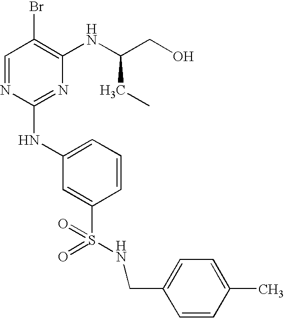 | 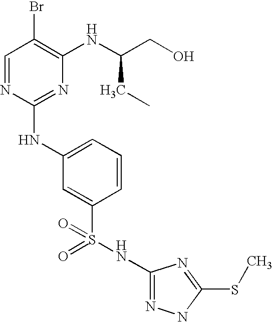 | 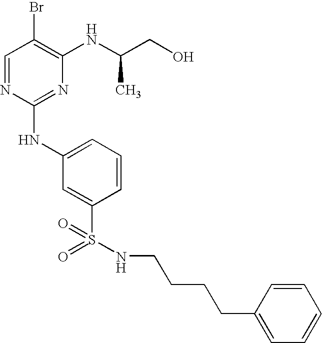 |
| Ex.No. | 405 | 406 | 407 | 408 |
| Molecular | 599.5283 | 520.4494 | 529.4419 | 534.4762 |
| Weight | | | | |
| ESI-MS | 599/601 | 520/522 | 529/531 | 534/536 |
| | 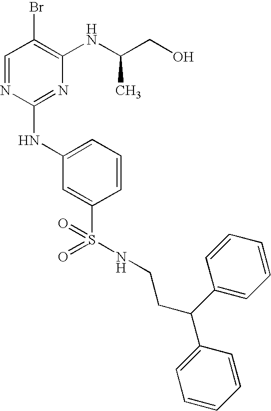 | 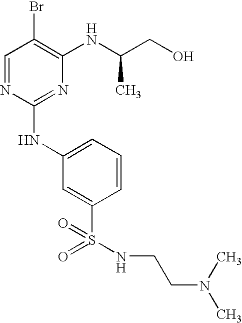 | 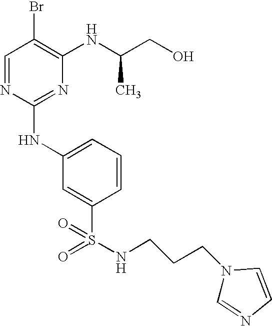 | 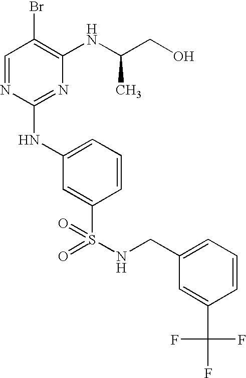 |
| Ex.No. | 409 | 410 | 411 | 412 |
| Molecular | 596.547 | 473.3935 | 510.4146 | 560.3929 |
| Weight | | | | |
| ESI-MS | 596/598 | 473/475 | 510/512 | 560/562 |
| | 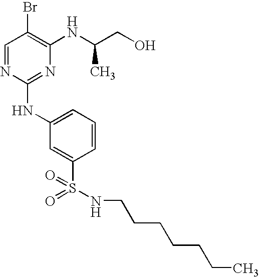 | 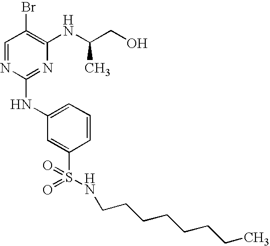 | 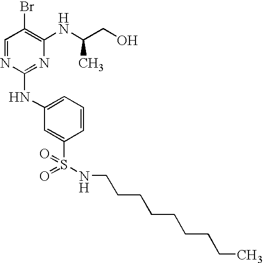 | 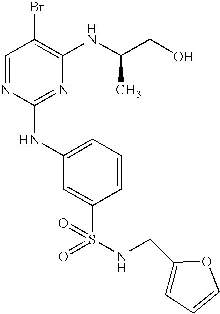 |
| Ex.No. | 413 | 414 | 415 | 416 |
| Molecular | 500.459 | 514.4858 | 528.5126 | 482.357 |
| Weight | | | | |
| ESI-MS | 500/502 | 514/516 | 528/530 | 482/484 |
| | 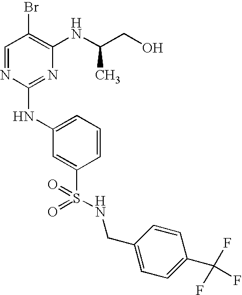 | 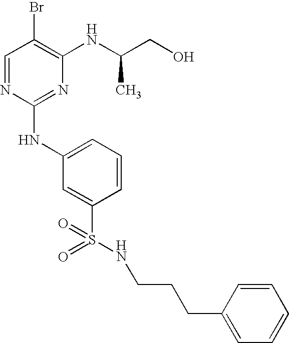 | 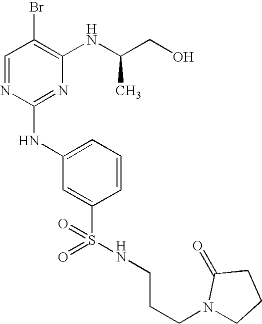 | 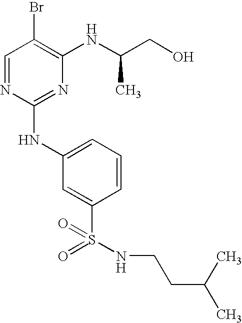 |
| Ex.No. | 417 | 418 | 419 | 420 |
| Molecular | 560.3929 | 520.4494 | 527.4413 | 472.4054 |
| Weight | | | | |
| ESI-MS | 560/562 | 520/522 | 527/529 | 472/474 |
| | 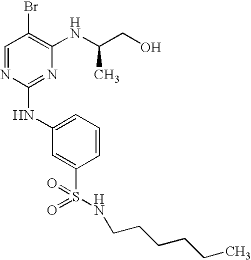 | 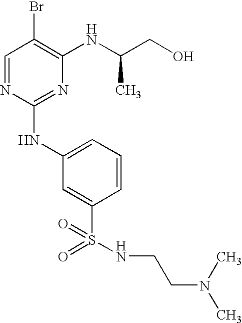 | 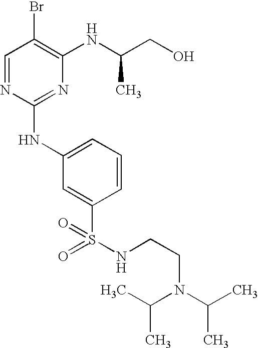 | 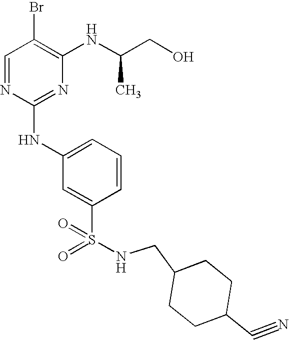 |
| Ex.No. | 421 | 422 | 423 | 424 |
| Molecular | 486.4322 | 487.4203 | 529.5007 | 523.4532 |
| Weight | | | | |
| ESI-MS | 486/488 | 487/489 | 529/531 | 523/525 |
| | 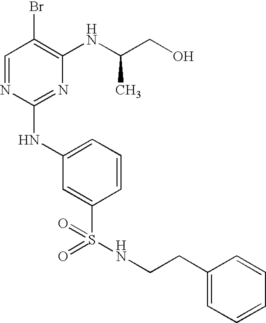 | 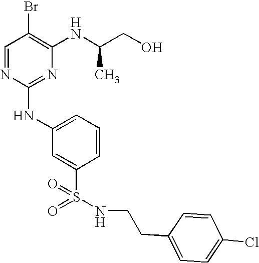 | 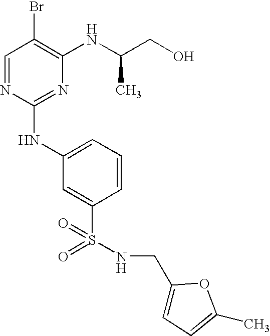 | 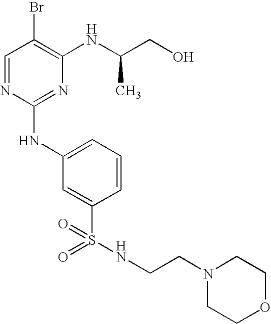 |
| Ex.No. | 425 | 426 | 427 | 428 |
| Molecular | 506.4226 | 540.8677 | 496.3838 | 515.4303 |
| Weight | | | | |
| ESI-MS | 506/508 | 540/542 | 496/498 | 515/517 |
| | 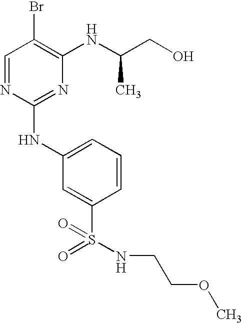 | 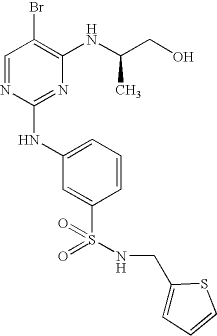 | 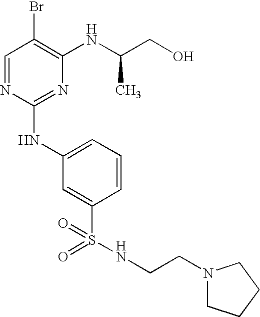 | 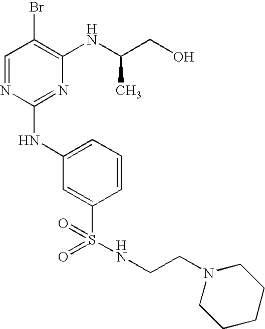 |
| Ex.No. | 429 | 430 | 431 | 432 |
| Molecular | 460.3508 | 498.424 | 499.4313 | 513.4581 |
| Weight | | | | |
| ESI-MS | 460/462 | 498/500 | 499/501 | 513/515 |
| | 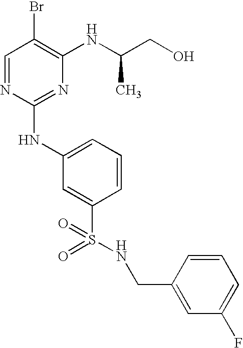 | 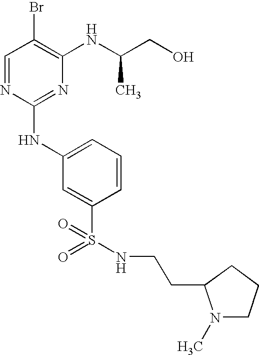 | 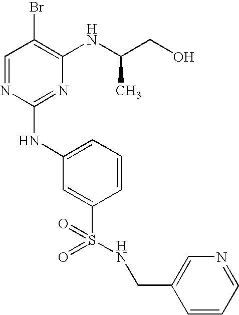 | 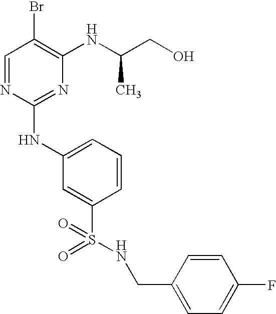 |
| Ex.No. | 433 | 434 | 435 | 436 |
| Molecular | 510.3859 | 513.4581 | 493.3839 | 510.3859 |
| Weight | | | | |
| ESI-MS | 510/512 | 513/515 | 493/495 | 510/512 |
| | 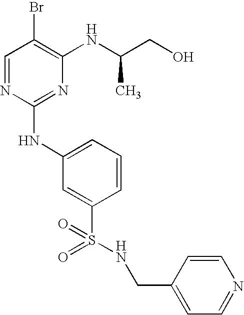 | 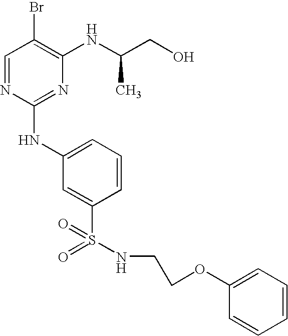 | 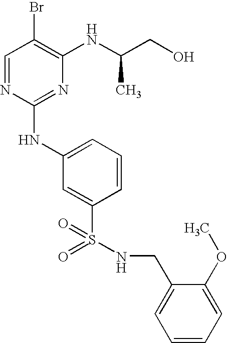 | 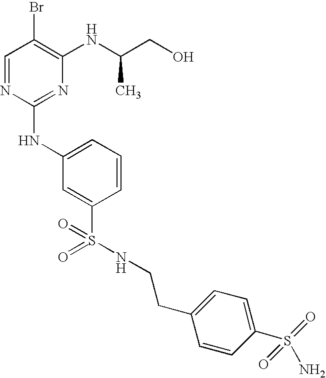 |
| Ex.No. | 437 | 438 | 439 | 440 |
| Molecular | 493.3839 | 522.4216 | 522.4216 | 585.5015 |
| Weight | | | | |
| ESI-MS | 493/495 | 522/524 | 522/524 | 585/587 |
| | 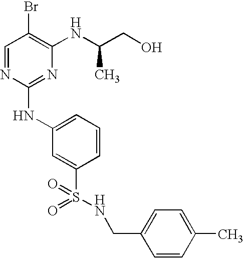 | 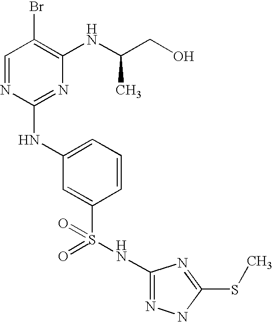 | 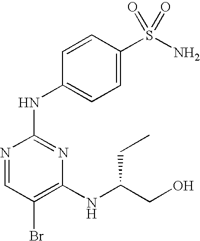 | |
| Ex.No. | 441 | 442 | 443 * | |
| Molecular | 506.4226 | 515.4151 | 416.30 | |
| Weight | | | | |
| ESI-MS | 506/508 | 515/517 | 416/418 | |

* Produced according to the process that is described under “Sulfonamides”

### Production of the Pyrimidine-Sulfonyl Fluorides of General Formula I

The production of pyrimidine-sulfonic acid fluorides is carried out analogously to the production of sulfonic acid amides.

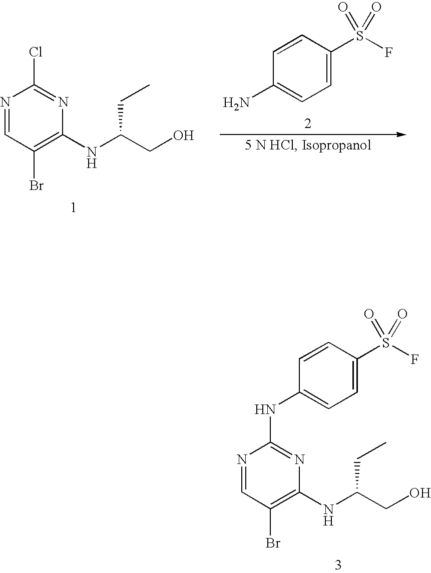

| | 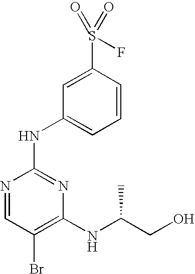 | 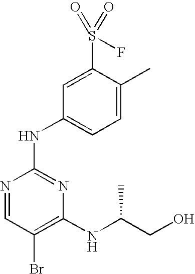 | 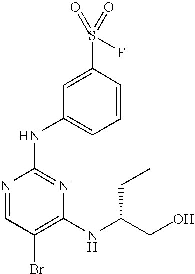 | 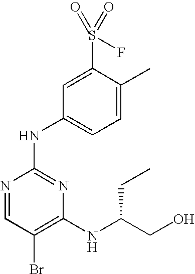 |
|---|---|---|---|---|
| Ex. No. | 444 | 445 | 446 | 447 |
| Molecular | 405.25 | 419.27 | 419.27 | 433.30 |
| Weight | | | | |
| ESI-MS | 217-220 | 196-202 | 165-196 | 198-204 |
| | 405/407 | 419/421 | 419/421 | 433/435 |
| | 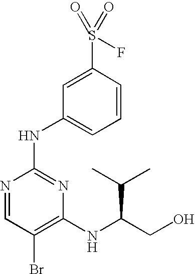 | 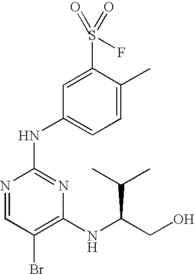 | 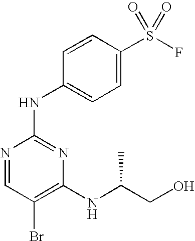 | 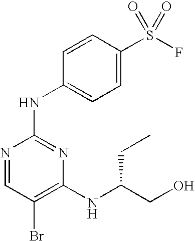 |
| Ex. No. | 448 | 449 | 450 | 451 |
| Molecular | 433.30 | 447.33 | 405.25 | 419.27 |
| Weight | | | | |
| ESI-MS | 144-149 | 219-222 | 170-173 | 226-228 |
| | 433/435 | 447/449 | 405/407 | 419/421 |
| | 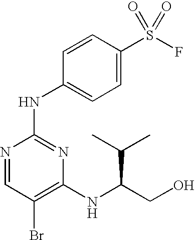 | 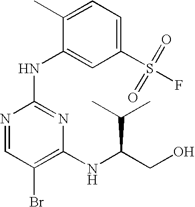 | 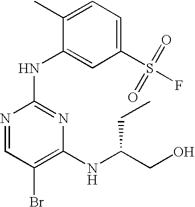 | 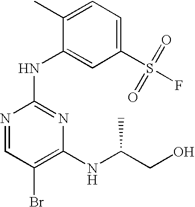 |
| Ex. No. | 452 | 453 | 454 | 455 |
| Molecular | 433.30 | 447.33 | 433.30 | 419.27 |
| Weight | | | | |
| ESI-MS | 433/435 | 447/449 | 433/435 | 419/421 |

Produced similarly to the above-described examples were also the following para-compounds:

| | 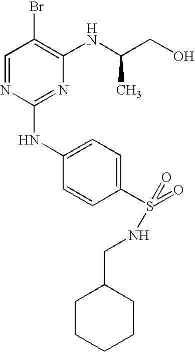 | 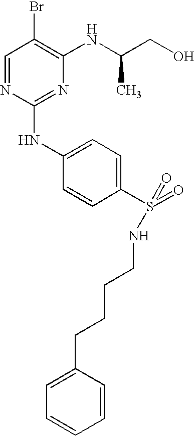 | 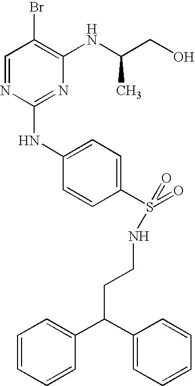 | 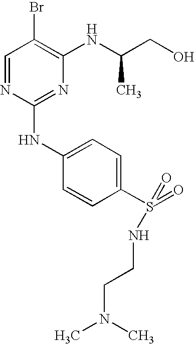 |
|---|---|---|---|---|
| Ex. No. | 456 | 457 | 458 | 459 |
| Molec- | 498.4432 | 534.4762 | 596.547 | 473.3935 |
| ular | | | | |
| Weight | | | | |
| ESI-MS | 498/500 | 534/536 | 596/598 | 473/475 |
| | 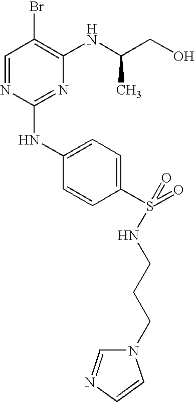 | 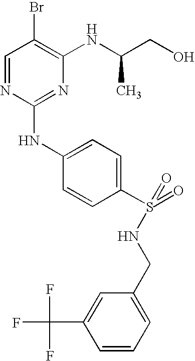 | 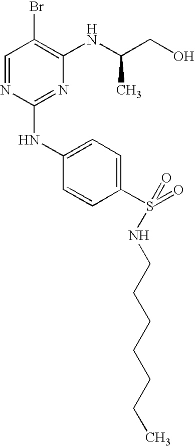 | 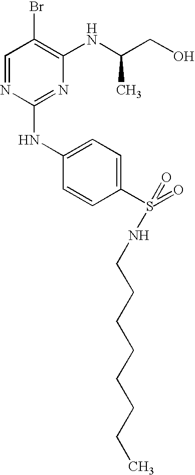 |
| Ex. No. | 460 | 461 | 462 | 463 |
| Molec- | 510.4146 | 560.3929 | 500.459 | 514.4858 |
| ular | | | | |
| Weight | | | | |
| ESI-MS | 510/512 | 560/562 | 500/502 | 514/516 |
| | 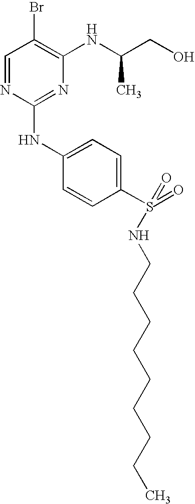 | 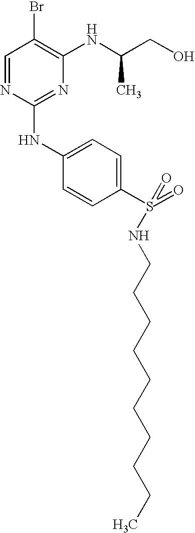 | 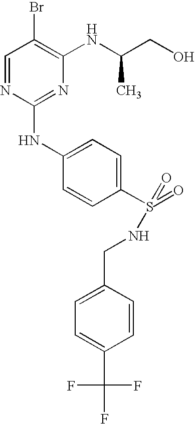 | 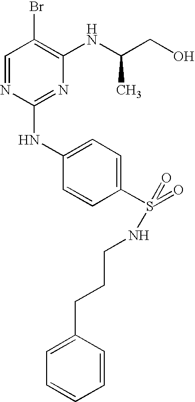 |
| Ex. No. | 464 | 465 | 466 | 467 |
| Molec- | 528.5126 | 542.5394 | 560.3929 | 520.4494 |
| ular | | | | |
| Weight | | | | |
| ESI-MS | 528/530 | 542/544 | 560/562 | 520/522 |
| | 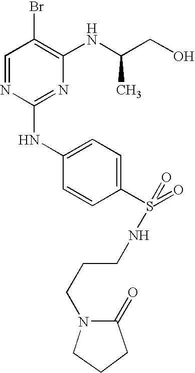 | 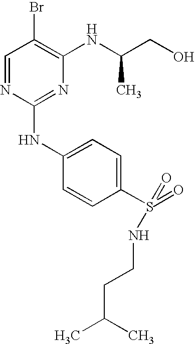 | 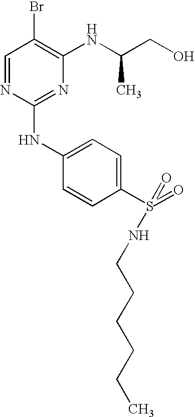 | 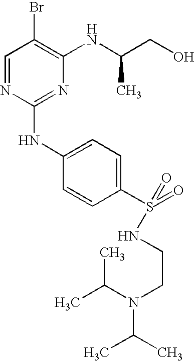 |
| Ex. No. | 468 | 469 | 470 | 471 |
| Molec- | 527.4413 | 472.4054 | 486.4322 | 529.5007 |
| ular | | | | |
| Weight | | | | |
| ESI-MS | 527/529 | 472/474 | 486/488 | 529/531 |
| | 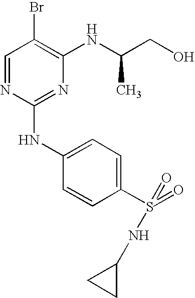 | 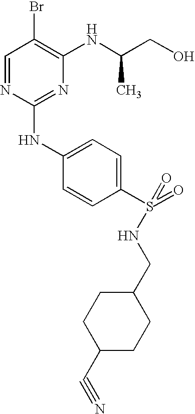 | 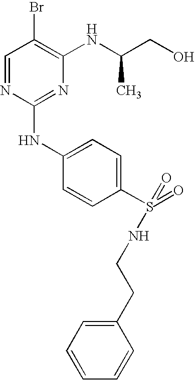 | 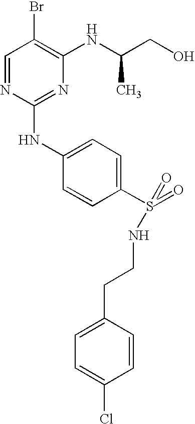 |
| Ex. No. | 472 | 473 | 474 | 475 |
| Molec- | 442.336 | 523.4532 | 506.4226 | 540.8677 |
| ular | | | | |
| Weight | | | | |
| ESI-MS | 442/444 | 523/525 | 506/508 | 540/542 |
| | 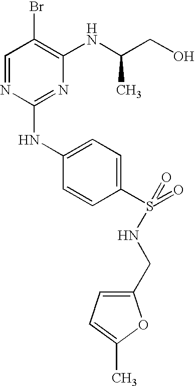 | 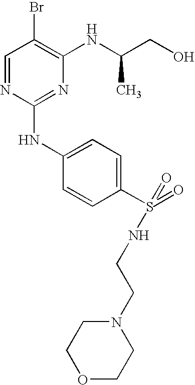 | 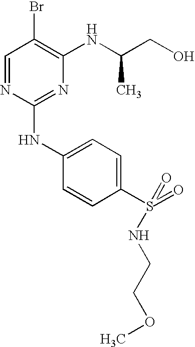 | 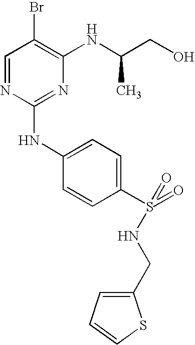 |
| Ex. No. | 476 | 477 | 478 | 479 |
| Molec- | 496.3838 | 515.4303 | 460.3508 | 498.424 |
| ular | | | | |
| Weight | | | | |
| ESI-MS | 496/498 | 515/517 | 460/462 | 498/500 |
| | 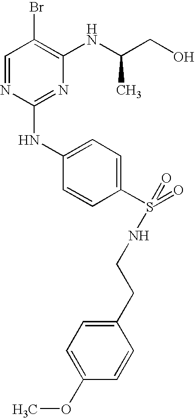 | 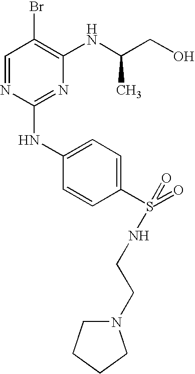 | 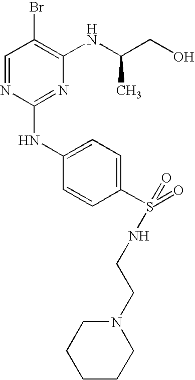 | 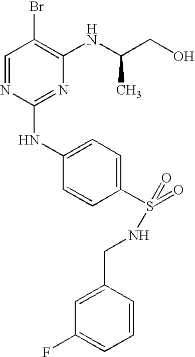 |
| Ex. No. | 480 | 481 | 482 | 483 |
| Molec- | 536.4484 | 499.4313 | 513.4581 | 510.3859 |
| ular | | | | |
| Weight | | | | |
| ESI-MS | 536/538 | 499/501 | 513/515 | 510/512 |
| | 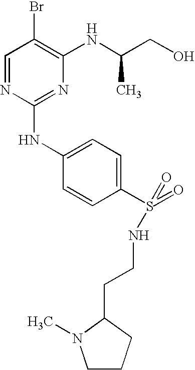 | 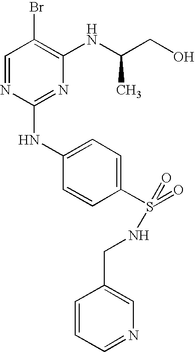 | 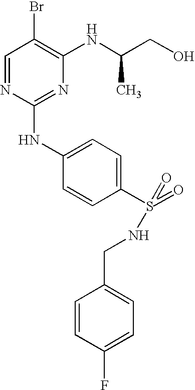 | 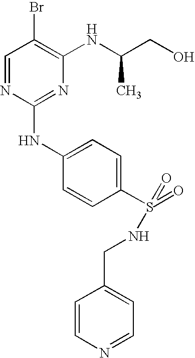 |
| Ex. No. | 484 | 485 | 486 | 487 |
| Molec- | 513.4581 | 493.3839 | 510.3859 | 493.3839 |
| ular | | | | |
| Weight | | | | |
| ESI-MS | 513/515 | 493/495 | 510/512 | 493/495 |
| | 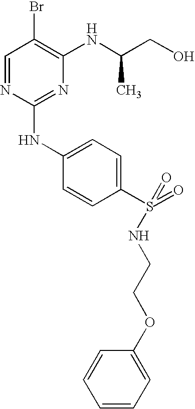 | 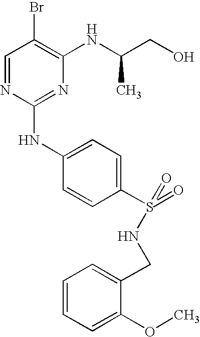 | 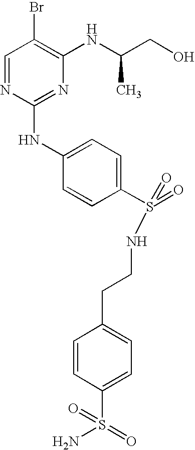 | 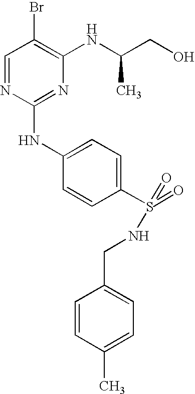 |
| Ex. No. | 488 | 489 | 490 | 491 |
| Molec- | 522.4216 | 522.4216 | 585.5015 | 506.4226 |
| ular | | | | |
| Weight | | | | |
| ESI-MS | 522/524 | 522/524 | 585/587 | 506/508 |

| | 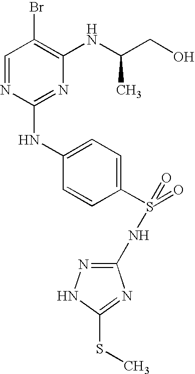 | 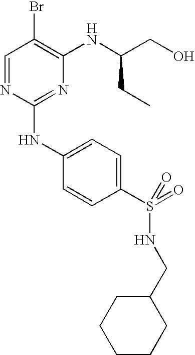 | 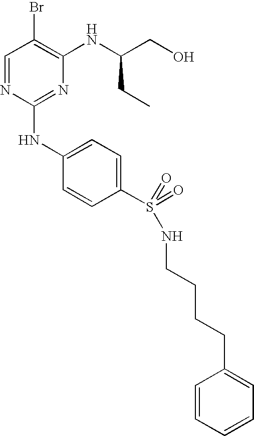 |
|---|---|---|---|
| Ex. No. | 492 | 493 | 494 |
| Molec- | 515.4151 | 512.47 | 548.503 |
| ular | | | |
| Weight | | | |
| ESI-MS | 515/517 | | |

| | 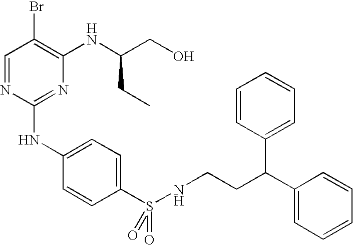 | 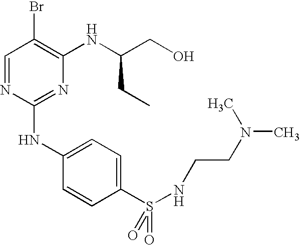 |
|---|---|---|
| Ex. No. | 495 | 496 |
| Molec- | 610.5738 | 487.4203 |
| ular | | |
| Weight | | |
| ESI-MS | | |

| | 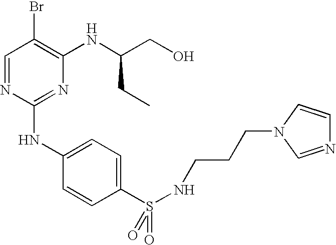 | 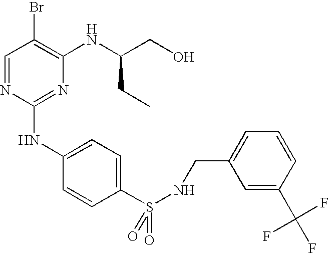 |
|---|---|---|
| Ex. No. | 497 | 498 |
| Molec- | 524.4414 | 574.4197 |
| ular | | |
| Weight | | |
| ESI-MS | | |

| | 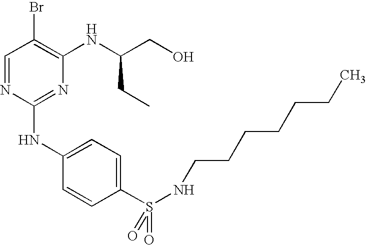 | 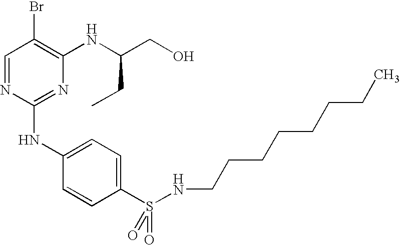 |
|---|---|---|
| Ex. No. | 499 | 500 |
| Molec- | 514.4858 | 528.5126 |
| ular | | |
| Weight | | |
| ESI-MS | | |

| | 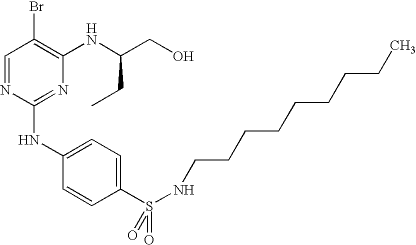 | 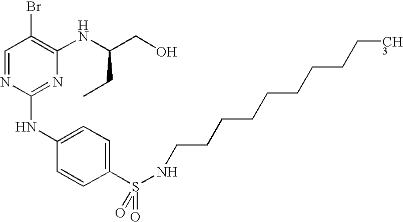 |
|---|---|---|
| Ex. No. | 501 | 502 |
| Molec- | 542.5394 | 556.5662 |
| ular | | |
| Weight | | |
| ESI-MS | | |

| | 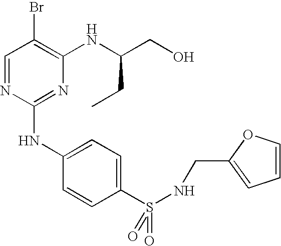 | 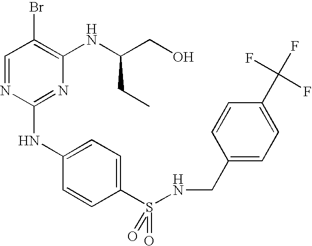 |
|---|---|---|
| Ex. No. | 503 | 504 |
| Molec- | 496.3838 | 574.4197 |
| ular | | |
| Weight | | |
| ESI-MS | | |

| | 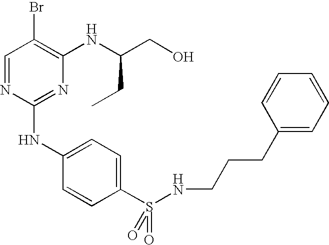 | 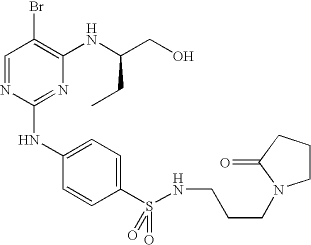 |
|---|---|---|
| Ex. No. | 505 | 506 |
| Molec- | 534.4762 | 541.4681 |
| ular | | |
| Weight | | |
| ESI-MS | | |

| | 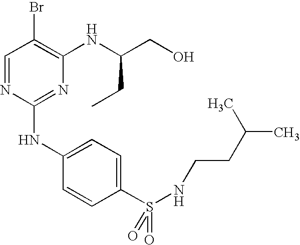 | 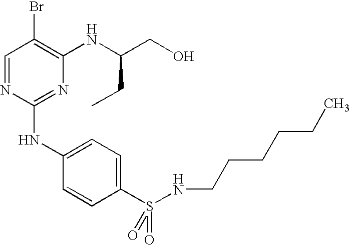 |
|---|---|---|
| Ex. No. | 507 | 508 |
| Molec- | 486.4322 | 500.459 |
| ular | | |
| Weight | | |
| ESI-MS | | |

| | 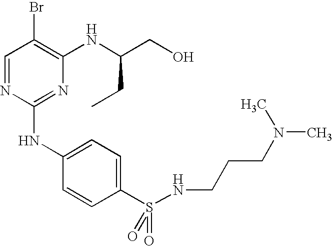 | 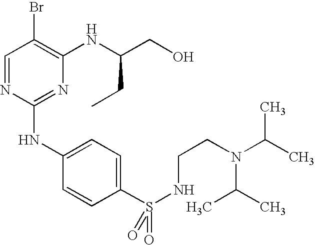 |
|---|---|---|
| Ex. No. | 509 | 510 |
| Molec- | 501.4471 | 543.5275 |
| ular | | |
| Weight | | |
| ESI-MS | | |

| | 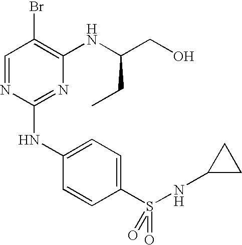 | 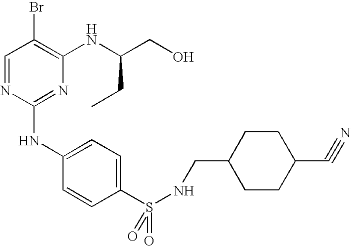 |
|---|---|---|
| Ex. No. | 511 | 512 |
| Molec- | 456.3628 | 537.4801 |
| ular | | |
| Weight | | |
| ESI-MS | | |

| | 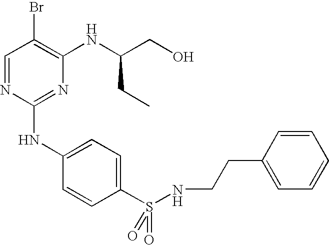 | 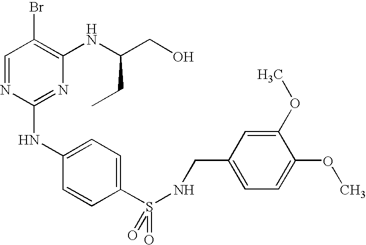 |
|---|---|---|
| Ex. No. | 513 | 514 |
| Molec- | 520.4494 | 566.4742 |
| ular | | |
| Weight | | |
| ESI-MS | | |

| | 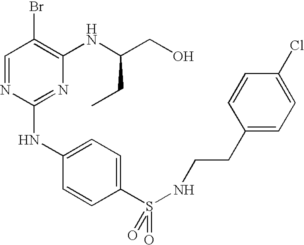 | 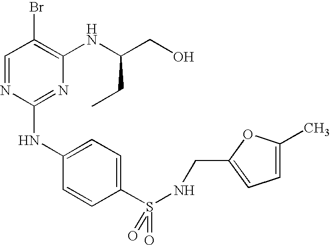 |
|---|---|---|
| Ex. No. | 515 | 516 |
| Molec- | 554.8945 | 510.4106 |
| ular | | |
| Weight | | |
| ESI-MS | | |

| | 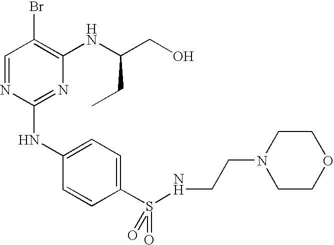 | 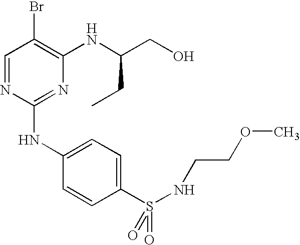 |
|---|---|---|
| Ex. No. | 517 | 518 |
| Molec- | 529.4571 | 474.3776 |
| ular | | |
| Weight | | |
| ESI-MS | | |

| | 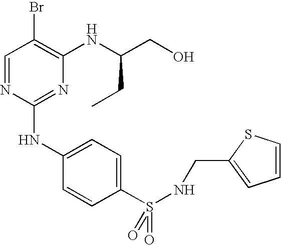 | 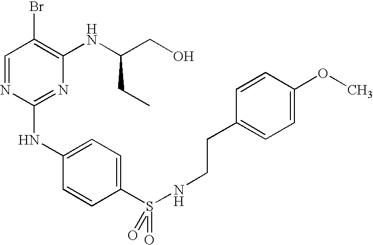 |
|---|---|---|
| Ex. No. | 519 | 520 |
| Molec- | 512.4508 | 550.4752 |
| ular | | |
| Weight | | |
| ESI-MS | | |

| | 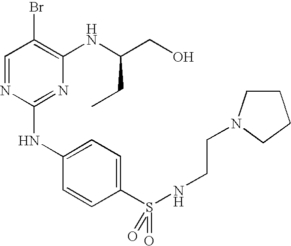 | 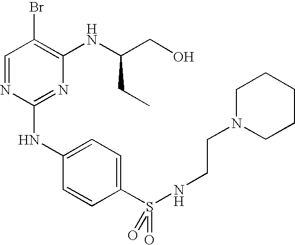 |
|---|---|---|
| Ex. No. | 521 | 522 |
| Molec- | 513.4581 | 527.4849 |
| ular | | |
| Weight | | |
| ESI-MS | | |

| | 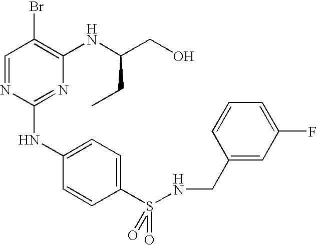 | 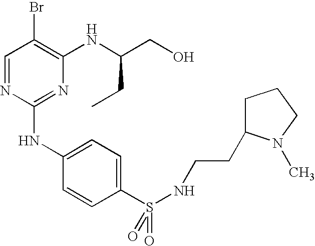 |
|---|---|---|
| Ex. No. | 523 | 524 |
| Molec- | 524.4127 | 527.4849 |
| ular | | |
| Weight | | |
| ESI-MS | | |

| | 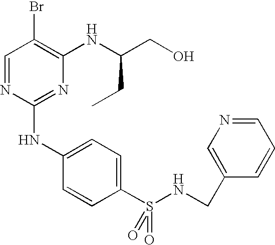 | 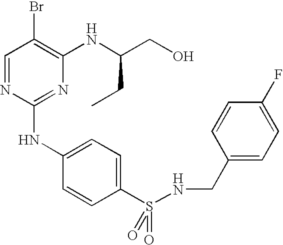 |
|---|---|---|
| Ex. No. | 525 | 526 |
| Molec- | 507.4107 | 524.4127 |
| ular | | |
| Weight | | |
| ESI-MS | | |

| | 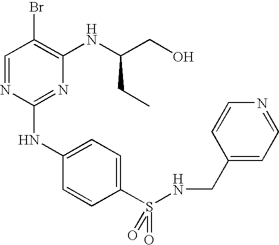 | 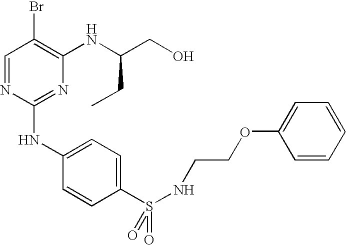 |
|---|---|---|
| Ex. No. | 527 | 528 |
| Molec- | 507.4107 | 536.4484 |
| ular | | |
| Weight | | |
| ESI-MS | | |

| | 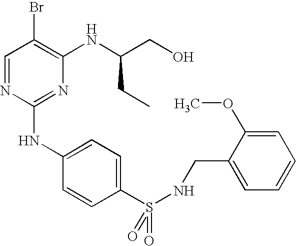 | 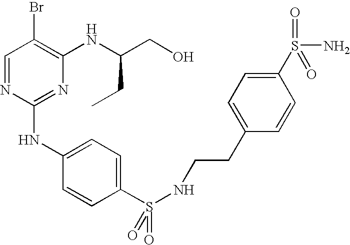 |
|---|---|---|
| Ex. No. | 529 | 530 |
| Molec- | 536.4484 | 599.5283 |
| ular | | |
| Weight | | |
| ESI-MS | | |

| | 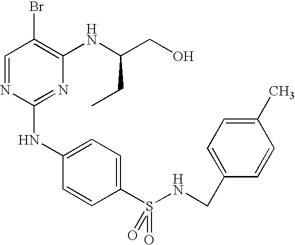 | 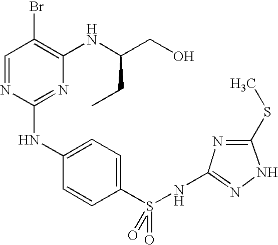 |
|---|---|---|
| Ex. No. | 531 | 532 |
| Molec- | 520.4494 | 529.4419 |
| ular | | |
| Weight | | |
| ESI-MS | | |

### Separation of Diastereomer Mixtures of the Compounds According to the Invention

Separation in the Example of the Diastereomer Mixture of Compound No. 274

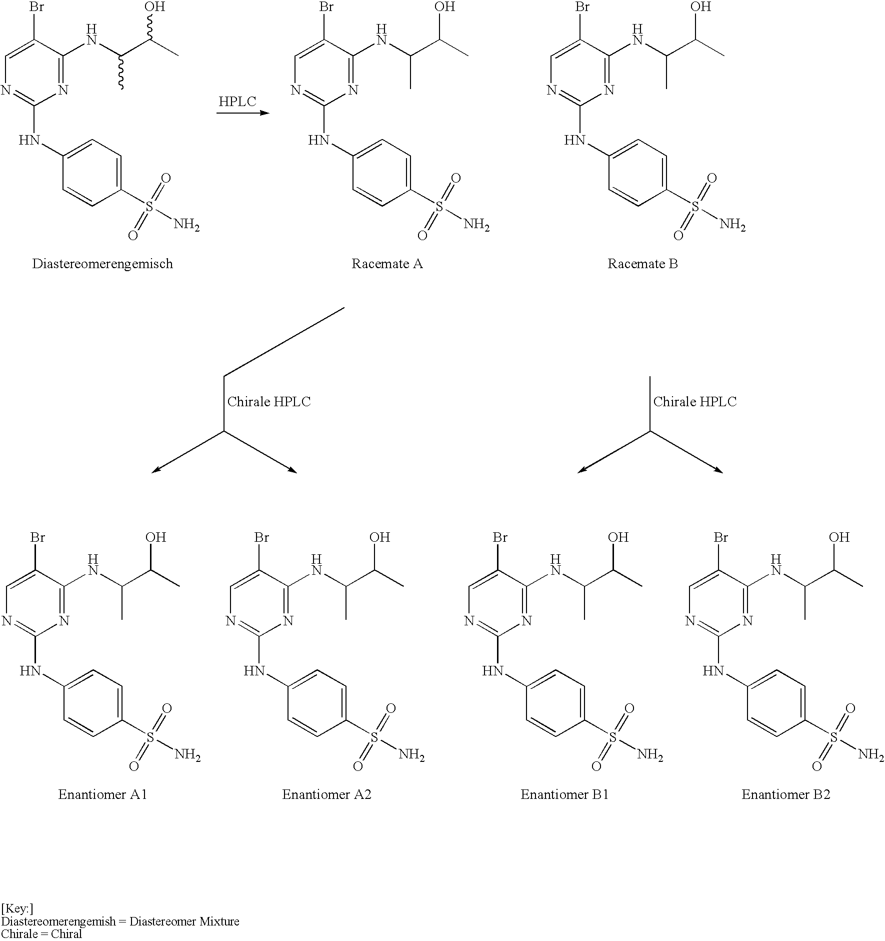

The diastereomer mixture was seperated into the two corresponding racemates (A and B) by means of HPLC.

Conditions

| | |
|---|---|
| Column: | Kromasil C18 (5 μm) 150 × 4.6 mm |
| Eluant: | 25% acetonitrile/water with 1 ml of NH3/1; |
| Flow: | 1.0 ml/min |
| Detection: | PDA 300 nm |
| Retention Times: | Racemate A - 11.6 minutes |
| | Racemate B - 12.4 minutes |

| | 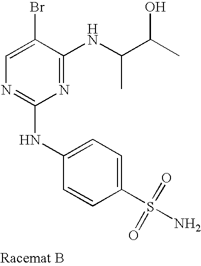 | 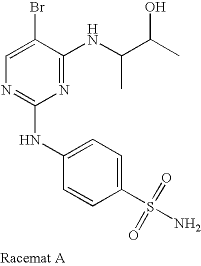 |
|---|---|---|
| NMR | DMSO-d6: | DMSO-d6: |
| | 9.68, s, 1H | 9.68, s, 1H |
| | 8.12, s, 1H | 8.11, s, 1H |
| | 7.87, d, 2H | 7.85, d, 2H |
| | 7.70, d, 2H | 7.69, d, 2H |
| | 7.14, s, 2H | 7.16, s, 2H |
| | 6.15, d, 1H | 6.35, d, 1H |
| | 5.01, d, 1H | 4.90, d, 1H |
| | 4.10, m, 1H | 4.08, m, 1H |
| | 3.80, m, 1H | 3.80, m, 1H |
| | 1.22, d, 3H | 1.18, d, 3H |
| | 1.1, d, 3H | 1.12, d, 3H |

Below, racemates A and B in each case were separated by means of chiral HPLC.

Conditions:

| | |
|---|---|
| Column: | Chiralpak AD (10 μm) 250 × 4.6 mm |
| Eluant: | Hexane/Ethanol 80:20 |
| Flow: | 1.0 ml/min |
| Detection: | PDA 300 nm |
| Retention Times: | Enantiomer A1 - 16.6 min |
| | Enantiomer A2 - 19.6 min |
| | Enantiomer B1 - 16.0 min |
| | Enantiomer B2 - 17.8 min |

The production of the intermediate stages that are preferably used for the synthesis of the compounds of general formula I according to the invention is described in WO 02/096888.

Another subject of this invention is also the use of compounds for birth control that fall under the industrial-property right DE 4029650 and whose action is in the fungicide range and that are described in the WO as CDK inhibitors. Use of this compound for birth control is not described to date.

| No. | Structure | No. | Structure | No. | Structure | | |
|---|---|---|---|---|---|---|---|
| 5 | 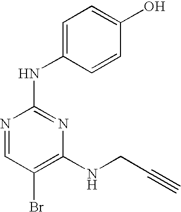 | 6 | 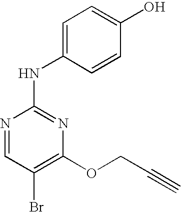 | 22 | 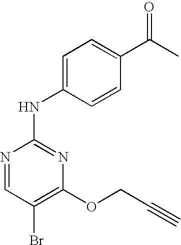 | 23 | 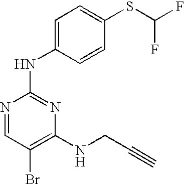 |
| 16 | 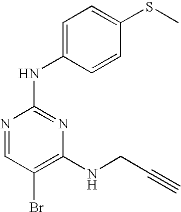 | 24 | 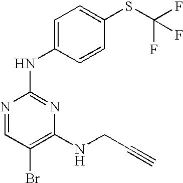 | 35 | 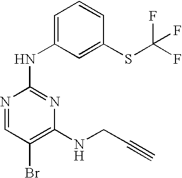 | 37 | 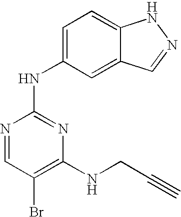 |
| 38 | 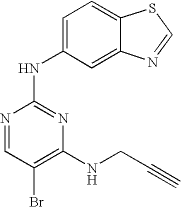 | 42 | 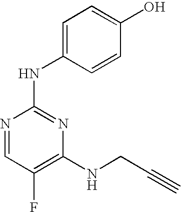 | 43 | 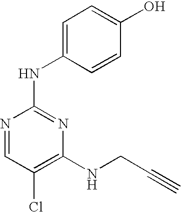 | 50 | 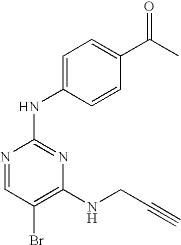 |
| 54 | 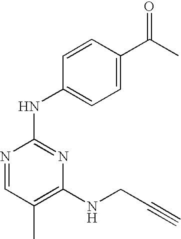 | 70 | 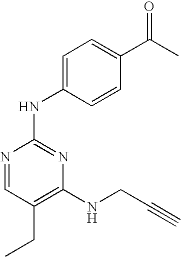 | 81 | 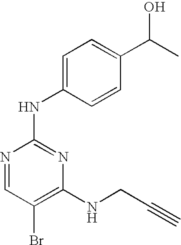 | 82 | 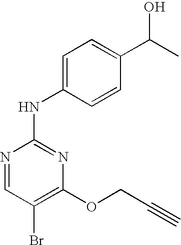 |

In addition, the invention thus relates to the use of pharmaceutical agents for birth control, comprising a compound of general formula I
in which
R¹ stands for halogen or C₁-C₃-alkyl,
X stands for oxygen or —NH,
A stands for hydrogen,
B stands for hydroxy, —CO-alkyl-R⁷, —S—CHF₂, —S—(CH₂)ₙCH(OH)CH₂N—R³R⁴, —S—CF₃, or —CH—(OH)—CH₃, or
A and B, independently of one another, can form a group

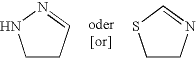

R², R³, R⁴, R⁷ and R⁸ have the meanings that are indicated in general formula I, as well as the isomers, diastereomers, enantiomers and salts.

The compounds according to the invention can be used for the production of a pharmaceutical agent for non-hormonal contraception.

In addition, a subject of this invention is the use of those compounds for birth control that fall under the industrial-property right U.S. Pat. No. 6,515,004 and also under the scope of protection of the application WO 01/44242 and whose action as inhibitors of the CDK-dependent kinases, whose use as agents for the production of a pharmaceutical agent for contraception is not disclosed to date, however, is known.

### EXAMPLE STRUCTURE

| Example | |
|---|---|
| Number | Structure |
| 533 | 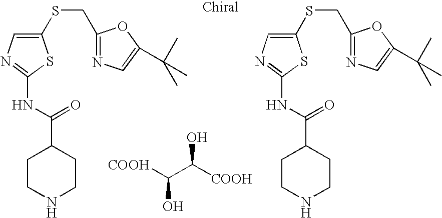 |

In addition, the invention thus relates to the use comprising a compound of general formula II 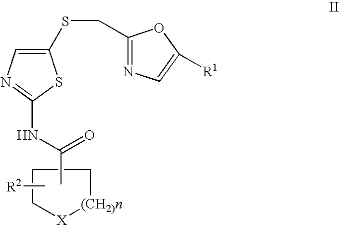
in which
R¹ stands for a C₁-C₁₀-alkyl group,
R² stands for hydrogen or alkyl,
X stands for NR₂ or CHNR₂R₃,
R¹ and R², independently of one another, stand for hydrogen, alkyl, substituted alkyl, cycloalkyl or substituted cycloalkyl, and
n=0, 1, 2, 3, as well as the isomers, enantiomers, diastereomers and salts thereof for the production of a pharmaceutical agent for non-horrnonal contraception.

The compounds have proven to be especially effective in which
R¹ stands for a C₁-C₁₀-alkyl group,
R² stands for hydrogen,
X stands for NR2 or CHNR₂R₃,
R¹ and R², independently of one another, stand for hydrogen, alkyl, substituted alkyl, cycloalkyl or substituted cycloalkyl, and
n=2, as well as the isomers, enantiomers, diastereomers and salts thereof.

The compound of formula Ia 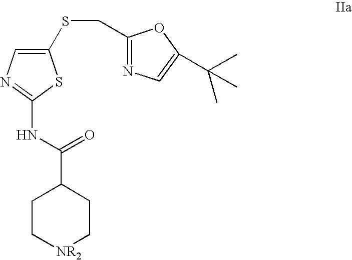
as well as the isomers, enantiomers, diastereomers and salts thereof and in which R² stands for hydrogen, alkyl, substituted alkyl or cycloalkyl, have proven to be quite especially effective.

### BIOLOGICAL EXAMPLES

1. Influence of CDK II Inhibitors on Spermatogenesis in Mice.

The tests are performed on adult male mice (mouse strain C57BL/6) with a weight of 25-30 g.

The animals are kept in Makrolon cages in spaces with controlled light (12 hours of darkness, 12 hours of light with a 30-minute twilight) and fed a standard diet (palletized SNIFF rat-mouse holder, 10 mm diameter) and supplied with as much tap water as they want.

1.1 Formulation and Administration of Test Substances:

The test substances are dissolved in dimethyl sulfoxide +0.9% common salt solution (1+1 v/v).

Altogether, 12 animals are treated with each test substance. In this case, they are divided into 3 groups that in each case obtained different amounts of test substances.

| | | |
|---|---|---|
| Group 1 | Vehicle (0.9% common salt solution 1 + 1 v/v)) | 4 Animals |
| Group 2 | 10 mg/kg/d of test substance | 4 Animals |
| Group 3 | 25 mg/kg/d of test substance | 4 Animals |

The test substance is administered subcutaneously over a period of 21 days via a mini-osmotic pump (Alza Company, Palo Alto, USA), Model 2002 (pump volume 0.5 μl/hour). The pumps must be changed after 14 days and replaced with new ones. They ensure a continuous administration of test substance.

1.2 Test Preparation:

The mini-osmotic pumps are stored for one day before the implantation with the test substances overnight in 0.9% common salt solution at 37° C. This step is important in order to ensure a uniform delivery.

The animals are anesthetized with diethyl ether. The fur on the back is disinfected with 70% ethanol and opened up with shears. With pointed forceps, a pocket is formed between the fur and back muscles in which the mini-osmotic pump is implanted. The wound is closed with Michel's sutures.

On day 21, the animals are anesthetized with diethyl ether, the abdominal cavity is opened up and exsanguinated by puncturing the body cavity veins. The blood serum that is obtained by centrifuging is used to determine LH, FSH and testosterone.

In the autopsy, thymus gland, spleen, testis, epididymis, seminal vesicles, prostate, liver and kidneys are prepared outside, and the weights are determined. The organs are worked up histologically (formalin-fixed and embedded in paraffin). Sections of the testes are stained with hematoxylin/eosin. In selected sections of the testes, in addition an apoptosis staining (protocol according to manufacturer information, Roche, Kit: In-Situ Cell Death Detection Kit AP, Cat. 1684809) is performed.

To determine sperm motility, the distal portion of an epididymis is removed (epididymis tail), cut, and transferred for 5 minutes in 500 μl of Dulbecco's PBS buffer that is 37° C. Then, the tissue portions are removed, and the motility of the sperm found in the buffer is examined by microscope.

To examine the sperm morphology, 20 μl of sperm suspension is dropped onto a slide and dried overnight. On the next day, an HE staining of the sperm smears is carried out.

To determine the sperm concentration, a 1:20 dilution (10 μl of parent suspension+190 μl of distilled water) is produced. To count the sperm in the Neugebauer counting chamber, 10 μl of suspension is used.

2. Inhibition of the Spontaneous Meiotic Maturation of Mouse Egg Cells

Egg cells are found before the so-called LH peak (luteinizing hormone), which injects the ovulation-triggering processes, in a state of meiotic arrest, which can be detected by the visibility of germinal vesicles (GV). If these egg cellsare isolated from the follicles that surround them, a spontaneous meiotic maturation sets in, since now inhibitory factors of the follicles are lacking, which otherwise maintain this meiotic arrest. Optically, the meiotic maturation is identified by the disappearance of the GV, the so-called germinal vesicle breakdown (GVB) and in the festering behavior in the ejection of the first polar body (PB).

In juvenile mice, the egg cell maturation is induced by injection of 10 IU of PMSG (Pregnant Mare Serum Gonadotrophin, i.p.). After 48 hours, the egg cells are isolated from antral follicles. In the medium, in which no specific inhibitors of meiosis are present, the spontaneous meiotic maturation to the GVB and PB stage begins. A measuring parameter is the influence of test substances on the spontaneous meiotic maturation.

3. Measurement of the Fertility of Female Rats:

Female rats have a stable four-day cycle. The cycle stages can be determined based on vaginal smears. If the animals in the estrus stage are bred with a male animal, in most cases a pregnancy results with 10-17 implantation sites.

Therefore, substances can be tested for their influence on fertility by the test substance being administered to female rats at different points in the cycle, being bred during estrus and e.g., the number of implantation sites being counted on day 16 after the breeding.

Test substances are administered once daily p.o., for example in a dose of 50 mg/kg over 4 days, beginning in the diestrus stage, whereby the animals in the estrus stage were bred. In this connection, there was a 35% reduction in the implantation sites, determined in the autopsy on day 16 after the breeding, relative to the vehicle control (8.2±6.1 versus 12.8±1.3; with n=6).

Test substances are administered twice daily, p.o., for example at a dose of 50 mg/kg over 2 days, beginning in the diestrus stage, whereby the animals in the estrus stage were bred. In this connection, there was a 56% reduction in the implantation sites, determined in the autopsy on day 16 after the breeding, relative to the vehicle control (6.0±3.5 versus 13.7±1.5; with n=6).

4. CDK 2/CycE Kinase Assay

Recombinant CDK2 and CycE-GST fusion proteins, purified from baculovirus-infected insect cells (Sf9), were obtained by Dr. Dieter Marmé, Klinik für Tumorbiologie [Clinic for Tumor Biology], Freiburg. Histone IIIS, which was used as a kinase substrate, was purchased from the Sigma Company.

CDK2/CycE (50 ng/measuring point) is incubated for 15 minutes at 22° C. in the presence of different concentrations of test substances (0 μm, as well as within the range of 0.01-100 μm) in assay buffer [50 mmol of tris/HCl, pH 8.0, 10 rnmol of MgCl2, 0.1 rnmol of Na ortho-vanadate, 1.0 mmol of dithiothreitol, 0.5 μm of adenosine trisphosphate (ATP), 10 μg/measuring point of histone IIIS, 0.2 μCi/measuring point of 33P-gamma ATP, 0.05% NP40, 1.25% dimethyl sulfoxide]. The reaction is halted by adding EDTA solution (250 mmol, pH 8.0, 14 μl/measuring point). From each reaction batch, 10 μl is applied to P30 filter strips (Wallac Company), and non-incorporated 33P-ATP was removed by subjecting the filter strips to three washing cycles for 10 minutes each in 0.5% phosphoric acid. After the filter strips are dried for 1 hour at 70° C., the filter strips are covered with scintillator strips (MeltiLex™ A, Wallac Company) and baked for 1 hour at 90° C. The amount of incorporated 33P (substrate phosphorylation) is determined by scintillation measurement in a gamma-radiation measuring device (Wallac).

### FIGURES

FIG. 1 shows a graphic visualization of the sperm concentration.

FIG. 2 shows a graphic visualization of the organ weights.

FIG. 3 shows a microscopic image of the mouse testis:
a. Histological section through a mouse testis of a vehicle-treated mouse (₂₀th lens)
b. 25 mg/kg/d of test substance (20^{th} lens); stars indicate the testicular tubuli that have a physiologically disturbed epithelium. Postmeiotic sperm stages do not seem to be present.
c. Mouse cauda epididymis (tail of the epididymis), 25 mg/kg/d of test substance (10^{th} lens). The arrows point to the spermatides that were released prematurely by testicular tubular lumen and were collected in the tail of the epididymis.

FIG. 4 shows the influence of a test substance on the spontaneous maturation of the egg cells (mouse). In this connection, the test substance was administered at a concentration of 0.1, 1 and 10 mmol; ethanol was used as a vehicle, n=8-10.

FIG. 5 shows the influence of a test substance on the fertility of female rats, 50 m/kg was administered twice daily. HP-β-CD (pH 5) was used as a vehicle.

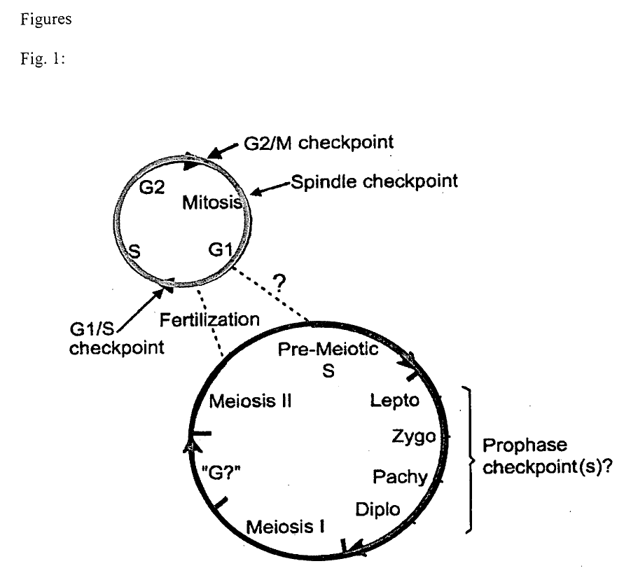

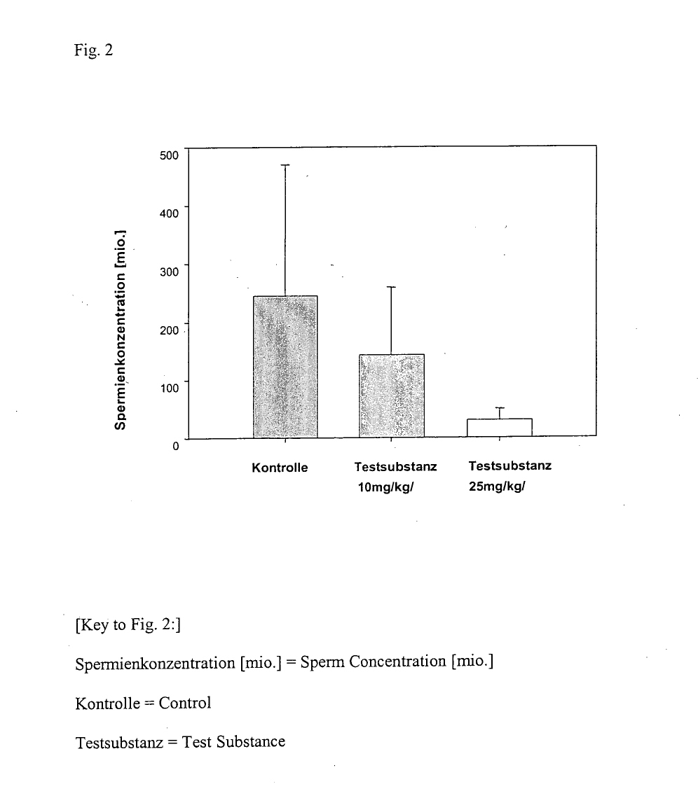

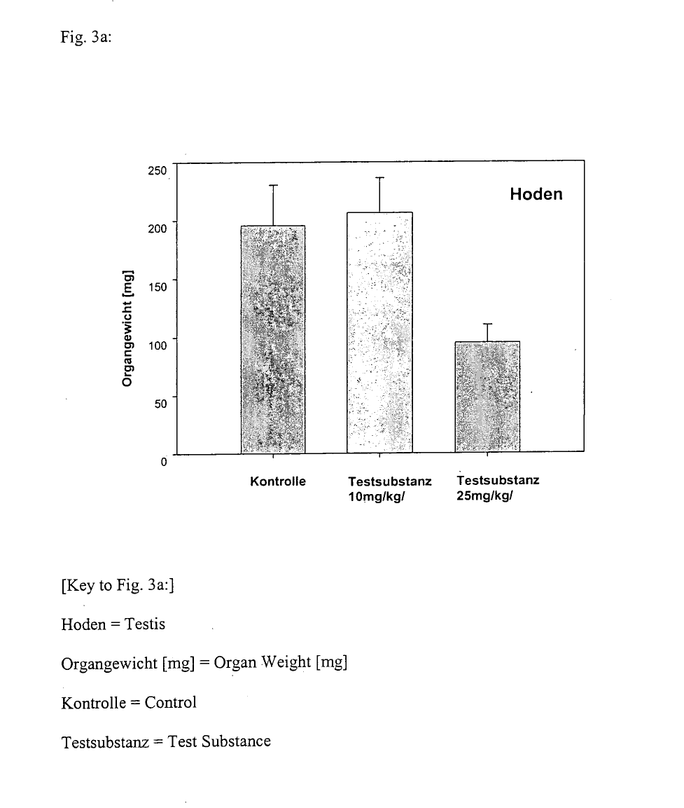

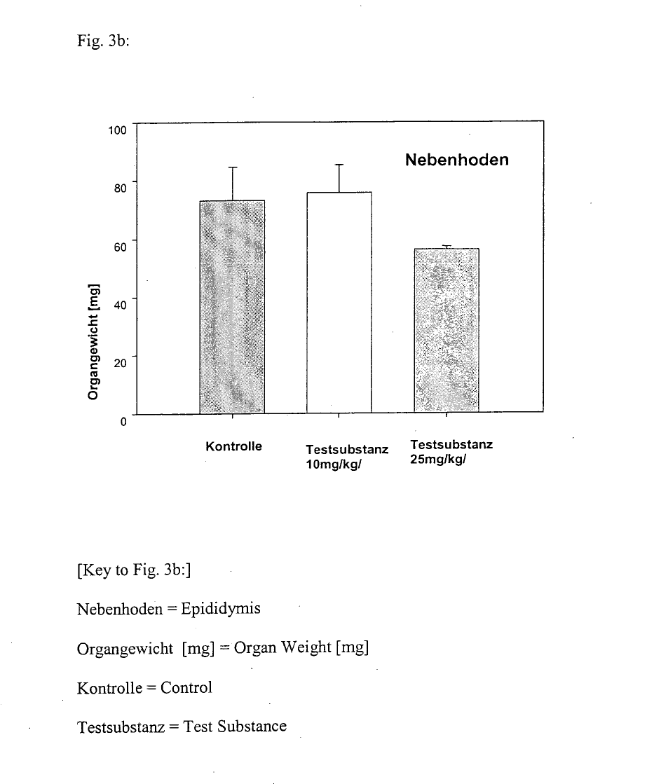

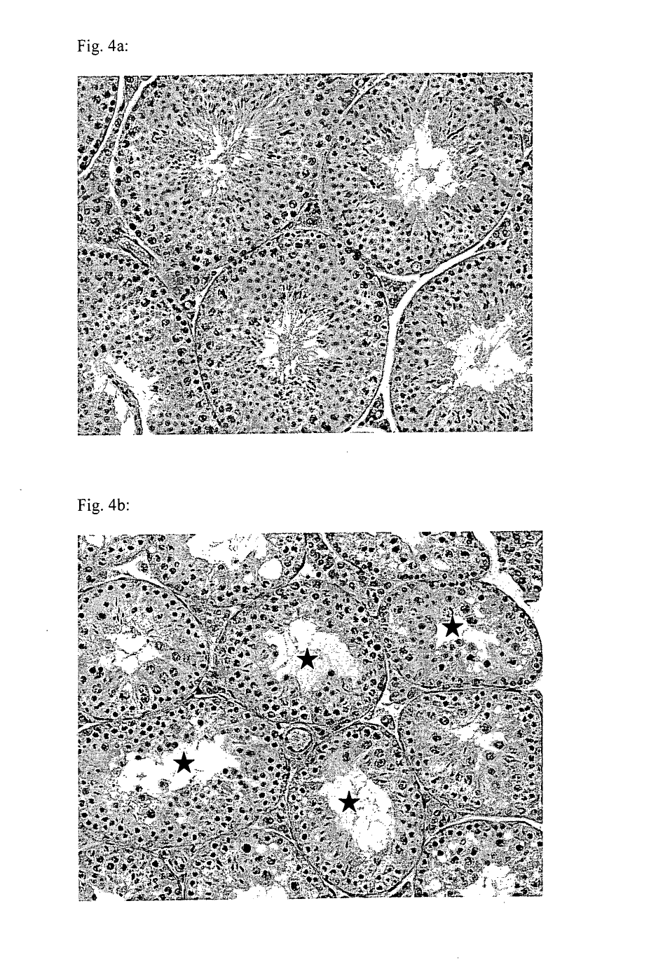

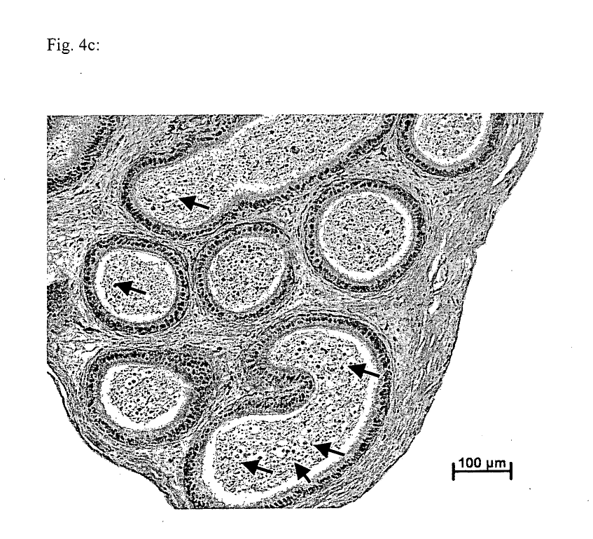

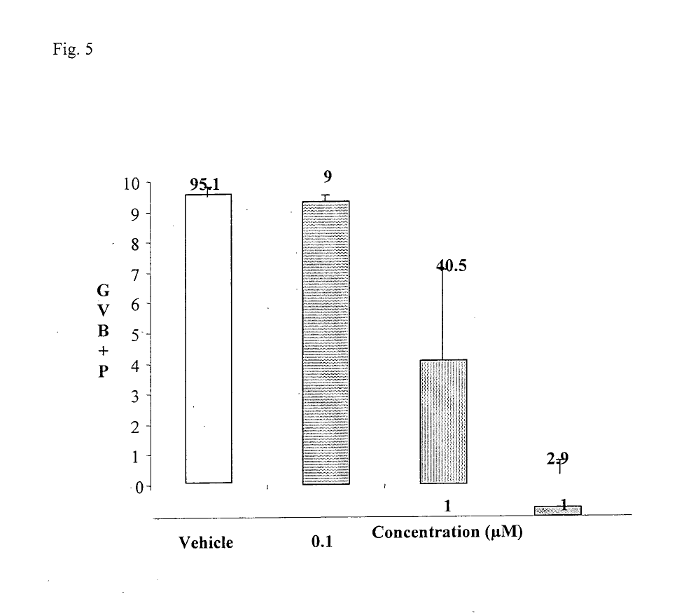

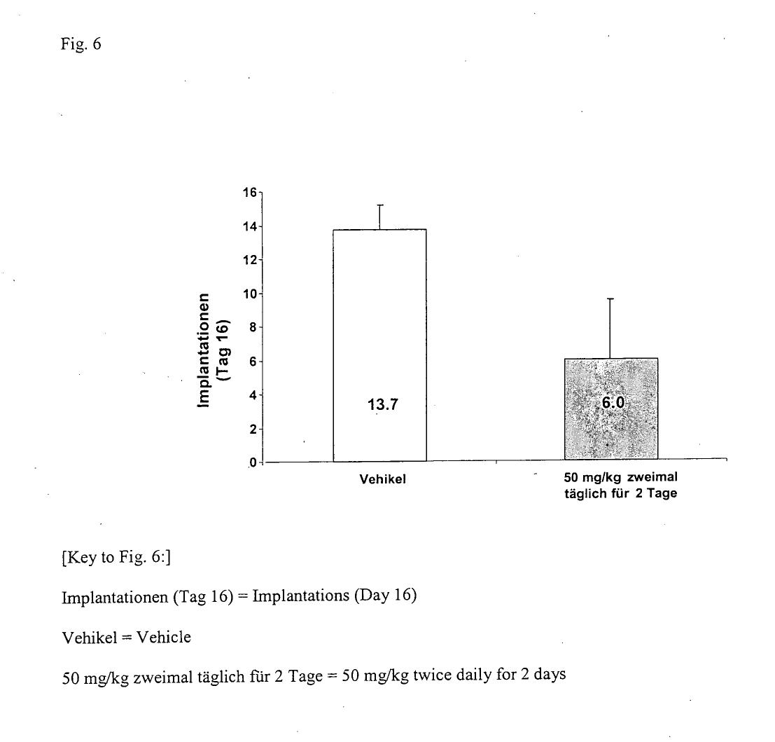

1. Method of birth control comprising the inhibition of CDK II.

2. Use of the compounds of general formula I 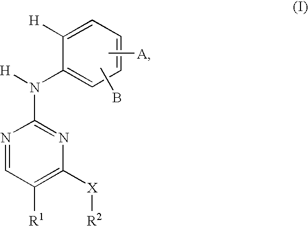
in which
R¹ stands for hydrogen, halogen, C₁-C₆-alkyl, nitro or for the group —COR⁵, —OCF₃, —(CH₂)ₙR⁵, —S—CF₃ or —SO₂CF₃,
R² stands for C₁-C₁₀-alkyl, C₂-C₁₀-alkenyl, C₂-C₁₀-alkinyl or C₃-C₁₀-cycloalkyl or for a C₁-C₁₀-alkyl, C₂-C₁₀-alkenyl, C₂-C₁₀-alkinyl or C₃-C₁₀-cycloalkyl that is substituted in one or more places, in the same way or differently, with hydroxy, halogen, C₁-C₆-alkoxy, C₁-C₆-alkylthio, armino, cyano, C₁-C₆-alkyl, —NH—(CH₂)ₙ—C₃-C₁₀-cycloalkyl, C₃-C₁₀-cycloalkyl, C₁-C₆-hydroxyalkyl, C₂-C₆-alkenyl, C₂-C₆-alkinyl, C₁-C₆-alkoxy-C₁-C₆-alkyl, C₁-C₆-alkoxy-C₁-C₆-alkoxy-C₁-C₆-alkyl, —NHC₁-C₆-alkyl, —N(C₁-C₆-alkyl)₂, —SO(C₁-C₆-alkyl), —SO₂(C₁-C₆-alkyl), C₁-C₆-alkanoyl, —CONR³R⁴, —COR⁵, C₁-C₆-alkylOAc, carboxy, aryl, heteroaryl, —(CH₂)ₙ-aryl, —(CH₂)ₙ-heteroaryl, phenyl-(CH₂)ₙ—R⁵, —(CH₂)ₙPO₃(R⁵)₂ or with the group —R⁶ or
—NR³R⁴, and the phenyl, C₃-C₁₀-cycloalkyl, aryl, heteroaryl, —(CH₂)ₙ-aryl and —(CH₂)ₙ-heteroaryl itself optionally can be substituted in one or more places, in the same way or differently, with halogen, hydroxy, C₁-C₆-alkyl, C₁-C₆-alkoxy, heteroaryl, benzoxy or with the group —CF₃ or —OCF₃, and the ring of C₃-C₁₀-cycloalkyl and the C₁-C₁₀-alkyl optionally can be interrupted by one or more nitrogen, oxygen and/or sulfur atoms, and/or can be interrupted by one or more ═C═O groups in the ring and/or optionally one or more possible double bonds can be contained in the ring, or
R² stands for the group 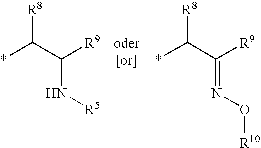
X stands for oxygen or for the group —NH—, —N(C₁-C₃-alkyl) or for —C₃-C₁₀-cycloalkyl, which can be substituted in one or more places, in the same way or differently, with a heteroaromatic compound, or
X and R² together form a C₃-C₁₀oycloalkyl ring, which optionally can contain one or more heteroatoms and optionally can be substituted in one or more places with hydroxy, C₁-C₆-alkyl, C₁-C₆-alkoxy or halogen,
A and B, in each case independently of one another, stand for hydrogen, hydroxy, C₁-C₃-alkyl, C₁-C₆-alkoxy or for the group —SR⁷, —S(O)R⁷, —SO₂R⁷, —NHSO₂R⁷, —CH(OH)R⁷, —CR⁷(OH)—R⁷, C₁-C₆-alkylP(O)OR³OR⁴, —COR⁷ or for 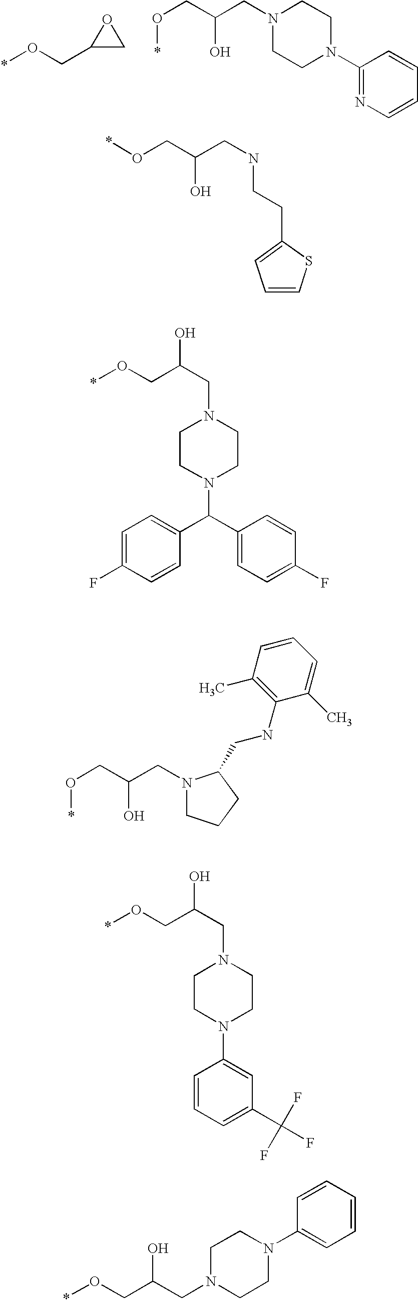 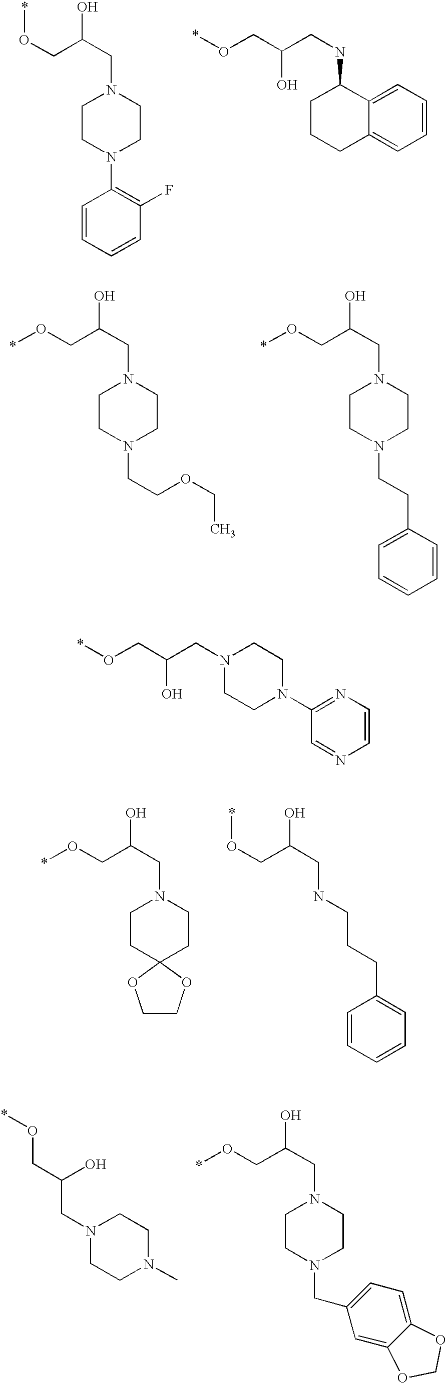 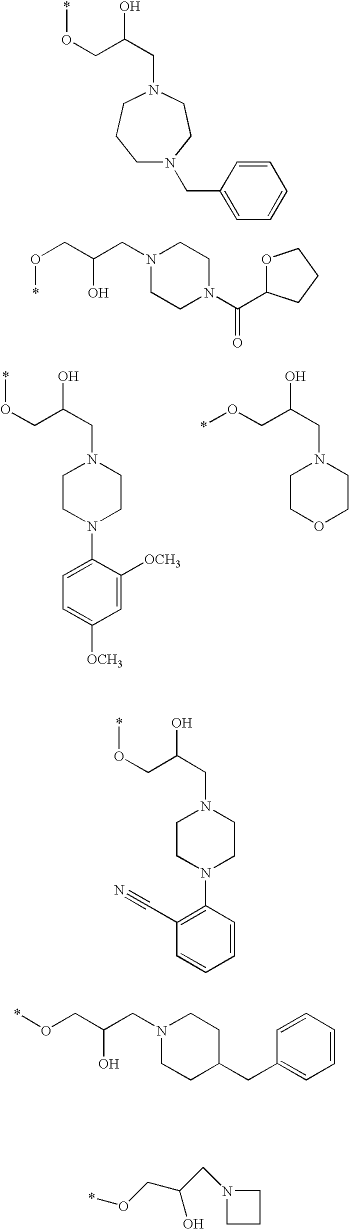
A and B together form a C₃-C₁₀-cycloalkyl ring that optionally can be interrupted by one or more nitrogen, oxygen and/or sulftir atoms and/or can be interrupted by one or more ═C═O or ═SO₂ groups in the ring, and/or optionally one or more possible double bonds can be contained in the ring, and the C₃-C₁₀-cycloalkyl ring optionally can be substituted in one or more places, in the same way or differently, with hydroxy, halogen, C₁-C₆-alkoxy, C₁-C₆-alkylthio, amino, cyano, C₁-C₆-alkyl, C₂-C₆-alkenyl, C₃-C₁₀-cycloalkyl, C₁-C₆-alkoxy-C₁-C₆-alkyl, —NHC₁-C₆-alkyl, —N(C₁-C₆-alkyl)₂, —SO(C₁-C₆-alkyl), —SO₂R⁷, C₁-C₆-alkanoyl, —CONR³R⁴, —COR⁵, C₁-C₆-alkylOAc, phenyl, or with the group R⁶, whereby the phenyl itself optionally can be substituted in one or more places, in the same way or differently, with halogen, hydroxy, C₁-C₆-alkyl, C₁-C₆-alkoxy, or with the group —CF₃ or —OCF₃,
R³ and R⁴, in each case independently of one another, stand for hydrogen, phenyl, benzyloxy, C₁-C₁₂-alkyl, C₁-C₆-alkoxy, C₂-C₄-alkenyloxy, C₃-C₆-cycloalkyl, hydroxy, hydroxy-C₁-C₆-alkyl, dihydroxy-C₁-C₆-alkyl, heteroaryl, heterocyclo-C₃-C₁₀-alkyl, heteroaryl-C₁-C₃-alkyl, C₃-C₆-cycloalkyl-C₁-C₃-alkyl that optionally is substituted with cyano, or for C₁-C₆-alkyl that optionally is substituted in one or more places, in the same way or differently, with phenyl, pyridyl, phenyloxy, C₃-C₆-cycloalkyl, C₁-C₆-alkyl or C₁-C₆-alkoxy, whereby the phenyl itself can be substituted in one or more places, in the same way or differently, with halogen, C₁-C₆-alkyl, C₁-C₆-alkoxy or with the group —SO₂NR³R⁴,
or for the group —(CH₂)ₙNR³R⁴, —CNHNH₂ or —NR³R⁴ or
R³ and R⁴ together form a C₃-C₁₀-cycloalkyl ring, which optionally can be interrupted by one or more nitrogen, oxygen and/or sulfur atoms, and/or can be interrupted by one or more ═C═O groups in the ring and/or optionally one or more possible double bonds can be contained in the ring,
R⁵ stands for hydroxy, phenyl, C₁-C₆-alkyl, C₃-C₆-cycloalkyl, benzoxy, C₁-C₆-alkylthio or C₁-C₆-alkoxy,
R⁶ stands for a heteroaryl or a C₃-C₁₀-cycloalkyl ring, whereby the ring has the above-indicated meaning,
R⁷ stands for halogen, hydroxy, phenyl, C₁-C₆-alkyl, C₂-C₆-alkenyl, C₂-C₆-alkinyl, or C₃-C₁₀-cycloalkyl with the above-indicated meaning, or for the group —NR³R⁴, or for C₁-C₁₀-alkyl, C₂-C₁₀-alkenyl, C₂-C₁₀-alkinyl or C₃-C₁₀-cycloalkyl that is substituted in one or more places, in the same way or differently, with hydroxy, C₁-C₆-alkoxy, halogen, phenyl, —NR³R⁴ or phenyl, which itself can be substituted in one or more places, in the same way or differently, with halogen, hydroxy, C₁-C₆-alkyl, C₁-C₆-alkoxy, halo-C₁-C₆-alkyl, or halo-C₁-C₆-alkoxy, or R⁷ stands for phenyl, which itself can be substituted in one or more places, in the same way or differently, with halogen, hydroxy, C₁-C₆-alkyl or C₁-C₆-alkoxy, halo-C₁-C₆-alkyl, or halo-C₁-C₆-alkoxy,
R⁸, R⁹ and
R¹⁰, in each case independently of one another, stand for hydrogen, hydroxy, C₁-C₁₀-alkyl, C₂-C₁₀-alkenyl, C₂-C₁₀-alkinyl, C₃-C₁₀-cycloalkyl, aryl, or heteroaryl, or for C₁-C₁₀-alkyl, C₂-C₁₀-alkenyl, C₂-C₁₀-alkinyl or C₃-C₁₀-cycloalkyl that is substituted in one or more places, in the same way or differently, with hydroxy, halogen, C₁-C₆-alkoxy, C₁-C₆-alkylthio, amino, cyano, C₁-C₆-alkyl, —NH—(CH₂)ₙ—C₃-C₁₀-cycloalkyl, C₃-C₁₀-cycloalkyl, C₁-C₆-hydroxyalkyl, C₂-C₆-alkenyl, C₂-C₆-alkinyl, C₁-C₆-alkoxy-C₁-C₆-alkyl, C₁-C₆-alkoxy-C₁-C₆-alkoxy-C₁-C₆-alkyl, —NHC₁-C₆-alkyl, —N(C₁-C₆-alkyl)₂, —SO(C₁-C₆-alkyl), —SO₂(C₁-C₆-alkyl), C₁-C₆-alkanoyl, —CONR³R⁴, —COR⁵, C₁-C₆-alkylOAc, carboxy, aryl, heteroaryl, —(CH₂)ₙ-aryl, —(CH₂)ₙ-heteroaryl, phenyl-(CH₂)ₙ—R⁵, —(CH₂)ₙPO₃(R⁵)₂ or with the group —R⁶ or
—NR³R⁴, and the phenyl, C₃-C₁₀-cycloalkyl, aryl, heteroaryl, —(CH₂)ₙ-aryl and —(CH₂)ₙ-heteroaryl itself optionally can be substituted in one or more places, in the same way or differently, with halogen, hydroxy, C₁-C₆-alkyl, C₁-C₆-alkoxy, or with the group —CF₃ or —OCF₃, and the ring of C₃-C₁₀-cycloalkyl and the C₁-C₁₀-alkyl optionally can be interrupted by one or more nitrogen, oxygen and/or sulfur atoms and/or can be interrupted by one or more ═C═O groups in the ring and/or optionally one or more possible double bonds can be contained in the ring, and
n stands for 0-6,
for the production of a pharmaceutical agent for contraception.

3. Use of the compounds according to claim 1
in which
R¹ stands for hydrogen, halogen, C₁-C₆-alkyl, nitro or for the group —COR⁵, —OCF₃, —(CH₂)ₙR⁵, —S—CF₃ or —SO₂CF₃,
R² stands for C₁-C₁₀-alkyl, C₂-C₁₀-alkenyl, C₂-C]₀-alkinyl or C₃-C₁₀-cycloalkyl or for C₁-C₁₀-alkyl, C₂-C₁₀-alkenyl, C₂-C₁₀-alkinyl or C₃-C₁₀-cycloalkyl that is substituted in one or more places, in the same way or differently, with hydroxy, halogen, C₁-C₆-alkoxy, C₁-C₆-alkylthio, amino, cyano, C₁-C₆-alkyl, —NH—(CH₂)ₙ—C₃-C₁₀-cycloalkyl, C₃-C₁₀-cycloalkyl, C₁-C₆-hydroxyalkyl, C₂-C₆-alkenyl, C₂-C₆-alkinyl, C₁-C₆-alkoxy-C₁-C₆-alkyl, C₁-C₆-alkoxy-C₁-C₆-alkoxy-C₁-C₆-alkyl, —NHC₁-C₆-alkyl, —N(C₁-C₆-alkyl)₂, —SO(C₁-C₆-alkyl), —SO₂(C₁-C₆-alkyl), C₁-C₆-alkanoyl, —CONR³R⁴, —COR⁵, C₁-C₆-alkylOAc, carboxy, aryl, heteroaryl, —(CH₂)ₙ-aryl, —(CH₂)ₙ-heteroaryl, phenyl-(CH₂)ₙ—R⁵, —(CH₂)ₙPO₃(R⁵)₂ or with the group —R⁶ or
—NR³R⁴, and the phenyl, C₃-C₁₀-cycloalkyl, aryl, heteroaryl, —(CH₂)ₙ-aryl and —(CH₂)ₙ-heteroaryl itself optionally can be substituted in one or more places, in the same way or differently, with halogen, hydroxy, C₁-C₆-alkyl, C₁-C₆-alkoxy, heteroaryl, benzoxy or with the group —CF₃ or —OCF₃, and the ring of C₃-C₁₀-cycloalkyl and C₁-C₁₀-alkyl optionally can be interrupted by one or more nitrogen, oxygen and/or sulfur atoms, and/or can be interrupted by one or more ═C═O groups in the ring and/or optionally one or more possible double bonds can be contained in the ring, or
R² stands for the group 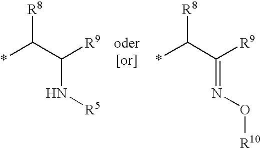
X stands for oxygen or for the group —NH—, —N(C₁-C₃-alkyl) or for —OC₃-C₁₀-cycloalkyl that can be substituted in one or more places, in the same way or differently, with a heteroaromatic compound, or
X and R² together form a C₃-C₁₀Icycloalkyl ring, which. optionally can contain one or more heteroatoms and optionally can be substituted in one or more places with hydroxy, C₁-C₆-alkyl, C₁-C₆-alkoxy or halogen,
A and B, in each case independently of one another, stand for hydrogen, hydroxy, C₁-C₃-alkyl, C₁-C₆-alkoxy or for the group —S—CH₃, —SO₂—C₂H₄—OH, —CO—CH₃, —S—CHF₂, —S—(CH₂)ₙ—CH(OH)CH₂N—R³R⁴, —CH₂P(O)OR³OR⁴, —S—CF₃, —SO—CH₃, —SO₂CF₃, —SO₂—(CH₂)ₙ—N—R³R⁴, —SO₂—NR³R⁴, —SO₂R⁷, —CH—(OH)—CH₃ or for 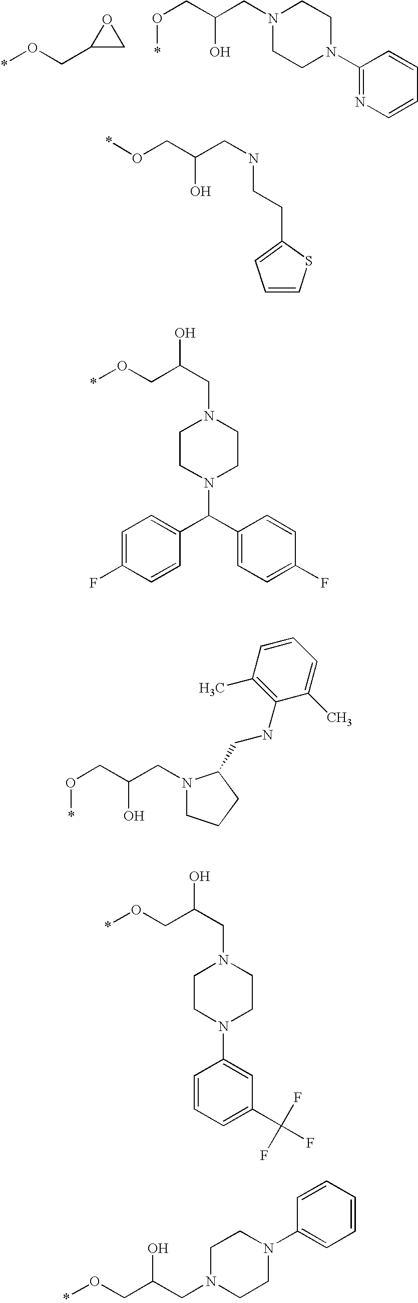 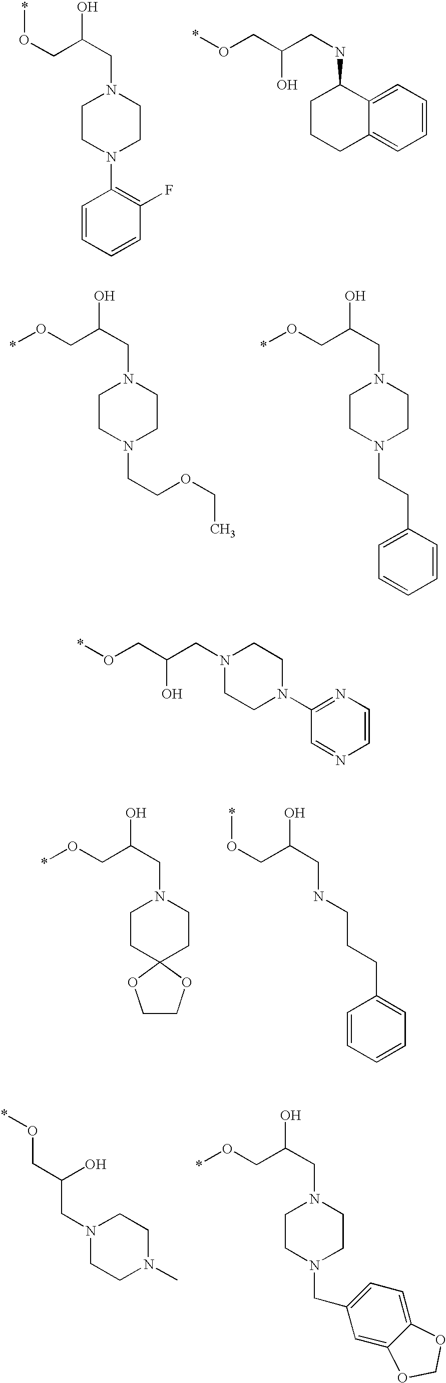 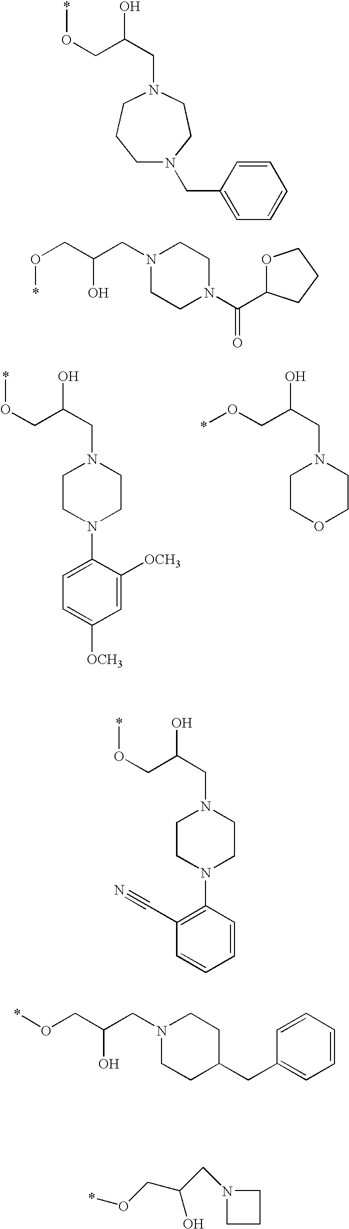
or
A and B together can form a group 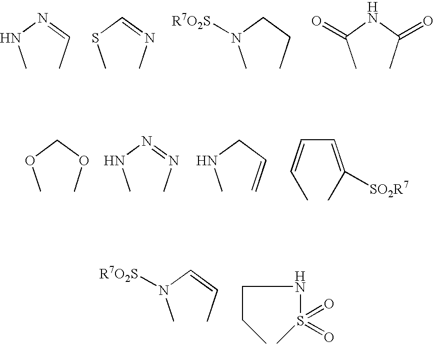
R³ and R⁴, in each case, independently of one another, stand for hydrogen, phenyl, benzyloxy, C₁-C₁₂-alkyl, C₁-C₆-alkoxy, C₂-C₄-alkenyloxy, C₃-C₆-cycloalkyl, hydroxy, hydroxy-C₁-C₆-alkyl, dihydroxy-C₁-C₆-alkyl, heteroaryl, heterocyclo-C₃-C₁₀-alkyl, heteroaryl-C₁-C₃-alkyl, C₃-C₆-cycloalkyl-C₁-C₃-alkyl that optionally is substituted with cyano, or for C₁-C₆-alkyl that optionally is substituted in one or more places, in the same way or differently, with phenyl, pyridyl, phenyloxy, C₃-C₆-cycloalkyl, C₁-C₆-alkyl or C₁-C₆-alkoxy, whereby the phenyl itself can be substituted in one or more places in the same way or differently with halogen, trifluoromethyl, C₁-C₆-alkyl, C₁-C₆-alkoxy or with the group —SO₂NR³R⁴,
or for the group —(CH₂)ₙNR³R⁴, —CNHNH₂ or —NR³R⁴ or for 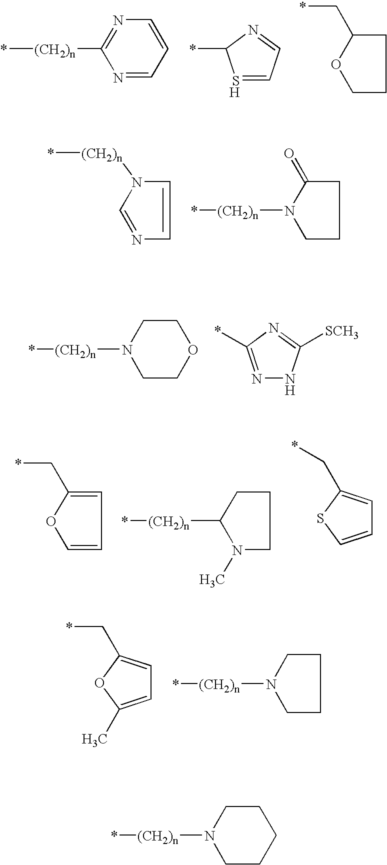
which optionally can be substituted with C₁-C₆-alkyl,
R⁵ stands for hydroxy, phenyl, C₁-C₆-alkyl, C₃-C₆-cycloalkyl, benzoxy, C₁-C₆-alkylthio or C₁-C₆-alkoxy,
R⁶ stands for the group 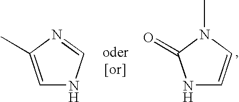
R⁷ stands for halogen, hydroxy, phenyl, C₁-C₆-alkyl, —C₂H₄OH, —NR³R⁴, or for the group 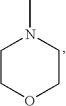
R⁸, R⁹ and
R¹⁰, in each case independently of one another, stand for hydrogen, hydroxy, C₁-C₆-alkyl, C₃-C₆-cycloalkyl or for the group 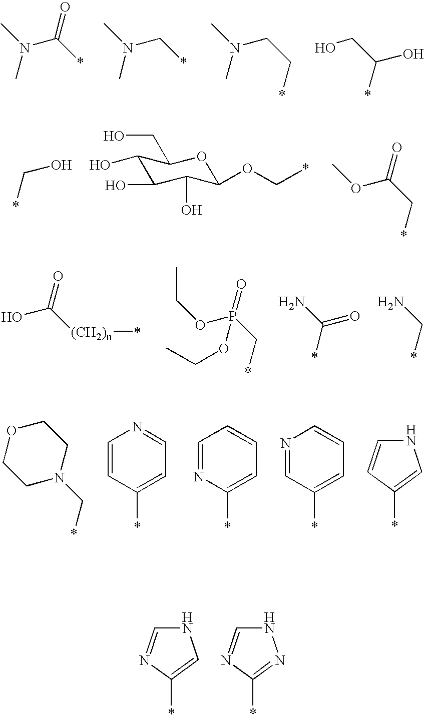
and
n stands for 0-6, as well as the isomers, enantiomers, diastereomers and salts thereof.

4. Use of the compounds according to claim 1, in which
R¹ stands for hydrogen, halogen, C₁-C₃-alkyl or for the group —(CH₂)ₙR⁵ steht,
R² stands for —CH(CH₃)—(CH₂)ₙ—R⁵, —CH—(CH₂OH)₂, —(CH₂)ₙR⁷, —CH(C₃H₇)—(CH₂)ₙ—R⁵, —CH(C₂H₅)—(CH₂)ₙ—R⁵, —CH₂—CN, —CH(CH₃)COCH₃, —CH(CH₃)—C(OH)(CH₃)₂, —CH(CH(OH)CH₃)OCH₃, —CH(C₂H₅)CO—R⁵, C₂-C₄-alkinyl, —(CH₂)ₙ—COR⁵, —(CH₂)ₙ—CO—C₁-C₆-alkyl, —(CH₂)ₙ—C(OH)(CH₃)-phenyl, —CH(CH₃)—C(CH₃)—R⁵, —CH(CH₃)—C(CH₃)(C₂H₅)—R⁵, —CH(OCH₃)—CH₂—R⁵, —CH₂—CH(OH)—R⁵, —CH(OCH₃)—CHR⁵—CH₃, —CH(CH₃)—CH(OH)—CH₂—CH═CH₂, —CH(C₂H₅)—CH(OH)—(CH₂)ₙ—CH₃, —CH(CH₃)—CH(OH)—(CH₂)ₙ—CH₃, —CH(CH₃)—CH(OH)—CH(CH₃)₂, (CH₂OAC)₂, —(CH₂)ₙ—R⁶, —(CH₂)ₙ—(CF₂)ₙ—CF₃, —CH(CH₂)ₙ—R⁵)₂, —CH(CH₃)—CO—NH₂, —CH(CH₂OH)-phenyl, —CH(CH₂OH)—CH(OH)—(CH₂)ₙR⁵, —CH(CH₂OH)—CH(OH)-phenyl, —CH(CH₂OH)—C₂H₄—R⁵, —(CH₂)ₙ—C≡C—C(CH₃)═CH—COR⁵, —CH(Ph)—(CH₂)ₙ—R⁵, —(CH₂)ₙ—COR⁵, —(CH₂)ₙPO₃(R⁵)₂, —(CH₂)ₙ—COR⁵, —CH((CH₂)ₙOR⁵)CO—R⁵, —(CH₂)ₙCONHCH((CH₂)ₙR⁵)₂, —(CH₂)ₙNH—COR⁵—CH(CH₂)ₙR⁵—(CH₂)ₙC₃-C₁₀-cycloalkyl, —(CH₂)ₙ—C₃-C₁₀-cycloalkyl, C₃-C₁₀-cycloalkyl; C₁-C₆-alkyl that optionally is substituted in one or more places, in the same way or differently, with hydroxy, C₁-C₆-alkyl or the group —COONH(CH₂)ₙCH₃ or —NR³R⁴; C₃-C₁₀-cycloalkyl, —(CH₂)ₙ—O—(CH₂)ₙ—R⁵, —(CH₂)ₙ—NR³R⁴, —CH(C₃H₇)—(CH₂)ₙ—OC(O)—(CH₂)—ₙ—CH₃, —(CH₂)—R⁵, —C(CH₃)₂—(CH₂)ₙ—R⁵—C(CH₂)ₙ(CH₃)—(CH₂)ₙR⁵, —C(CH₂)ₙ—(CH₂)ₙR⁵, —CH(t-butyl)-(CH₂)ₙ—R⁵, —CCH₃(C₃H₇)—(CH₂)ₙR⁵, —CH(C₃H₇)—(CH₂)ₙ—R⁵, —CH(C₃H₇)—COR⁵, —CH(C₃H₇)—(CH₂)ₙ—OC(O)—NH—Ph, —CH((CH₂)ₙ(C₃H₇))—(CH₂)ₙR⁵, —CH(C₃H₇)—(CH₂)ₙ—OC(O)—NH—Ph(OR⁵)₃, —NR³R⁴, —NH—(CH₂)ₙ—NR³R⁴, R⁵—(CH₂)ₙ—C*H—CH(R⁵)—(CH₂)ₙ—R⁵, —(CH₂)ₙ—CO—NH—(CH₂)ₙ—CO—R⁵, —OC(O)NH—C₁-C₆-alkyl or —(CH₂)ₙ—CO—NH—(CH₂)ₙ—CH—((CH₂)ₙR⁵)₂,
or for C₃-C₁₀-cycloalkyl, which is substituted with the group 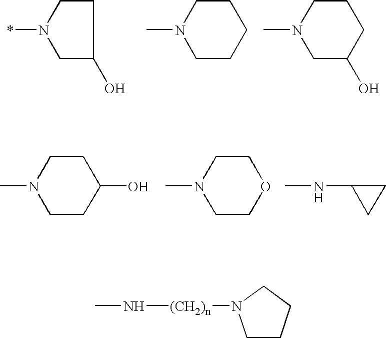
or for the group 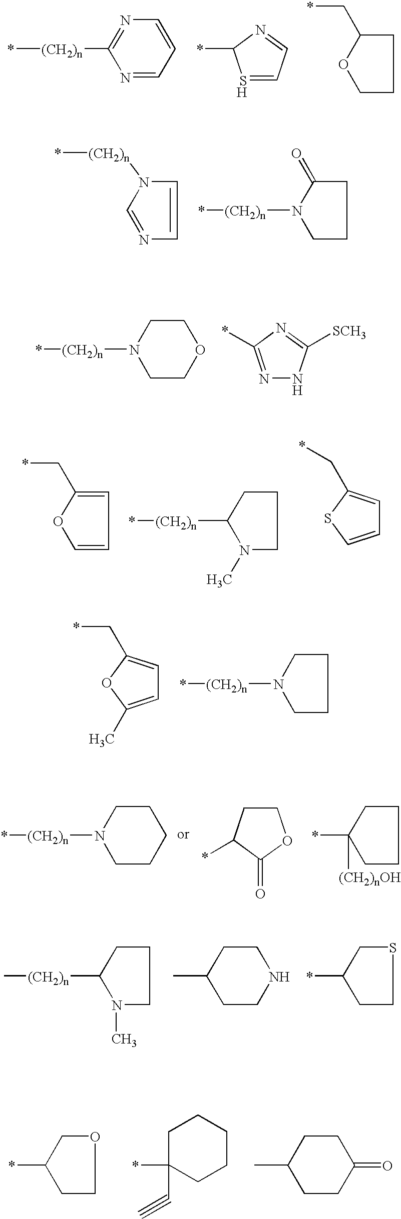
X stands for oxygen or for the group —NH, —N(C₁-C₃-alkyl) or 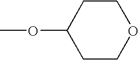
or
R² stands for the group 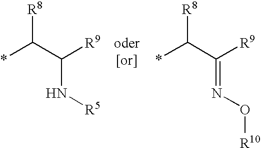
or
X and R² together form a group 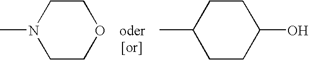
A and B, in each case independently of one another, stand for hydrogen, hydroxy, C₁-C₃-alkyl, C₁-C₆-alkoxy or for the group —S—CH₃, —SO₂—C₂H₄—OH, —CO—CH₃, —S—CHF₂, —S—(CH₂)ₙCH(OH)CH₂N—R³R⁴, —CH₂PO(OC₂H₅)₂, —S—CF₃, —SO—CH₃, —SO₂CF₃, —SO₂—(CH₂)ₙ—N—R³R⁴, —SO₂—NR³R⁴, —SO₂R⁷, —CH(OH)—CH₃, —COOH, —CH((CH₂)ₙR⁵)₂, —(CH₂)ₙR⁵, —COO—C₁C₆-alkyl, —CONR³R⁴ or for 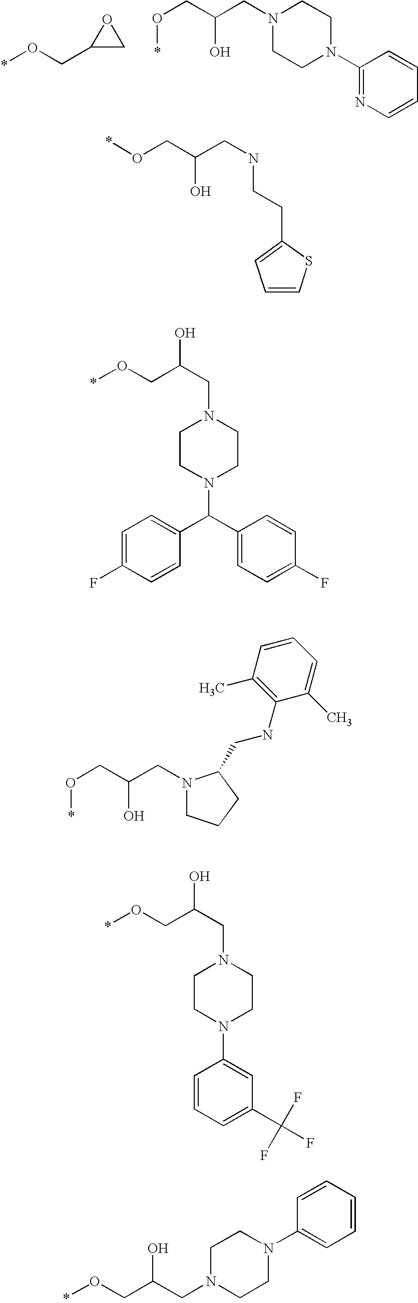 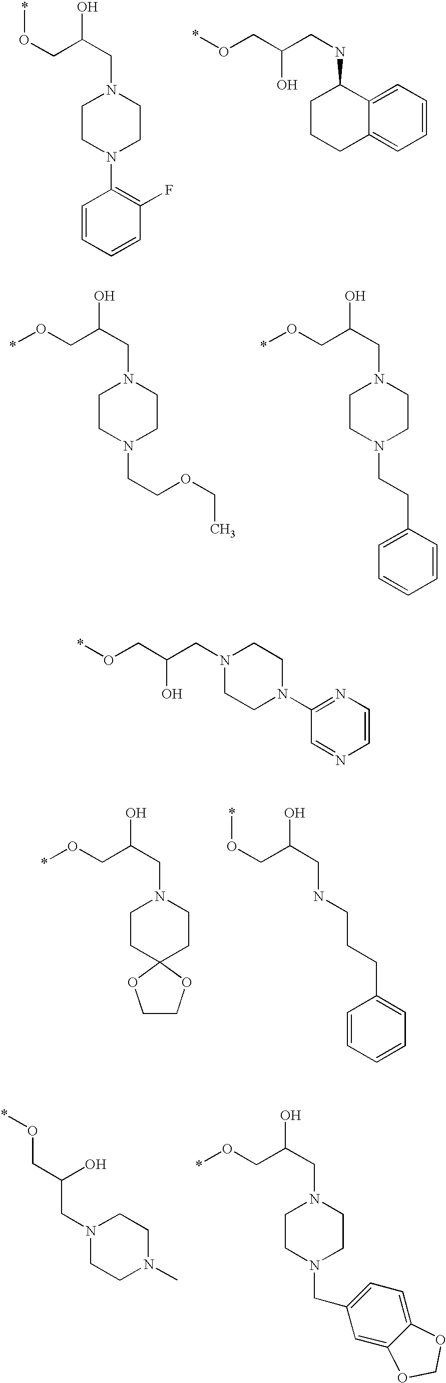 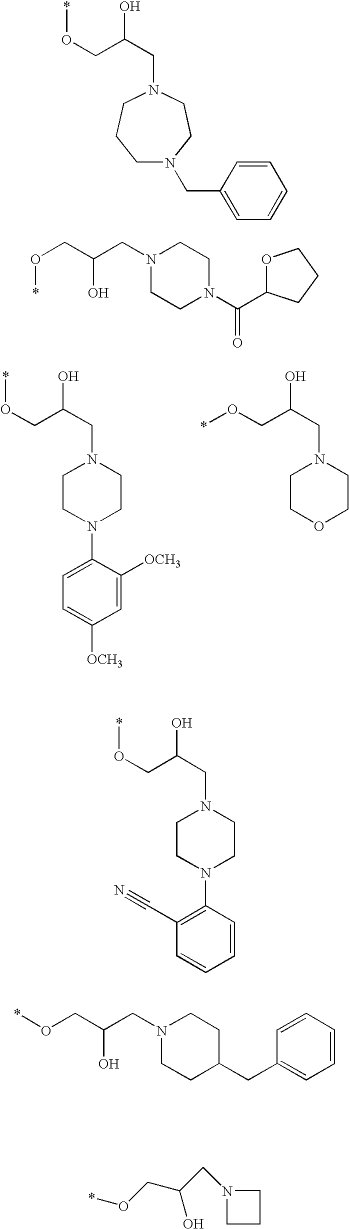
A and B together can form a group 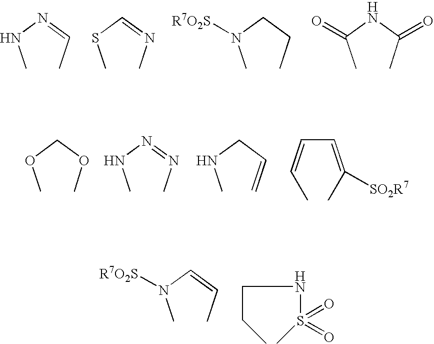
R³ and R⁴, in each case independently of one another, stand for hydrogen, phenyl, benzyloxy, C₁-C₁₂-alkyl, C₁-C₆-alkoxy, C₂-C₄-alkenyloxy, C₃-C₆-cycloalkyl, hydroxy, hydroxy-C₁-C₆-alkyl, dihydroxy-C₁-C₆-alkl, heteroaryl, beterocyclo-C₃-C₁₀-alkyl, heteroaryl-C₁-C₃-alkyl, C₃-C₆-cycloalkyl-C₁-C₃-alkyl that optionally is substituted with cyano, or for C₁-C₆-alkyl that optionally is substituted in one or more places, in the same way or differently, with phenyl, pyridyl, phenyloxy, C₃-C₆-cycloalkyl, C₁-C₆-alkyl or C₁-C₆-alkoxy, whereby the phenyl itself can be substituted in one or more places, in the same way or differently, with halogen, trifluoromethyl, C₁-C₆-alkyl, C₁-C₆-alkoxy or with the group —SO₂NR³R⁴, or for the group —(CH₂)ₙNR³R⁴, —CNHNH₂ or —NR³R⁴ or for 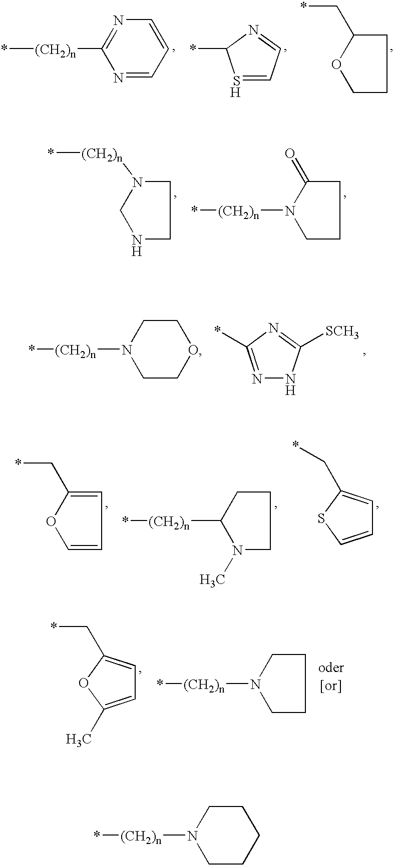
which optionally can be substituted with C₁-C₆-alkyl,
R⁵ stands for hydroxy, phenyl, C₁-C₆-alkyl, C₃-C₆-cycloalkyl, benzoxy, C₁-C₆-alkylthio or C₁-C₆-alkoxy,
R⁶ stands for the group 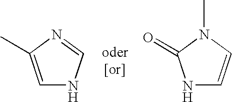
R⁷ stands for halogen, hydroxy, phenyl, C₁-C₆-alkyl, —(CH₂)ₙOH, —NR³R⁴ or the group 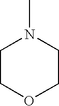
R⁸, R⁹ and
R¹⁰ stand for hydrogen, hydroxy, C₁-C₆-alkyl or for the group —(CH₂)ₙ—COOH, and
n stands for 0-6,
as well as the isomers, diastereomers, enantiomers and salts thereof.

5. Use of the compounds comprising general formula I, in which
R¹ stands for halogen or C₁-C₃-alkyl,
X stands for oxygen or —NH,
A stands for hydrogen,
B stands for hydroxy, —CO-alkyl-R⁷, —S—CHF₂, —S—(CH₂)ₙCH(OH)CH₂N—R³R⁴, —S—CF₃, or —CH—(OH)—CH₃, or
A and B, independently of one another, can form a group 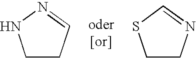
R², R³, R⁴, R⁷ and R⁸ have the meanings that are indicated in general formula I, as well as the isomers, diastereomers, enantiomers and salts for the production of a pharmaceutical agent for contraception.

6. Use of the compounds comprising general formula II 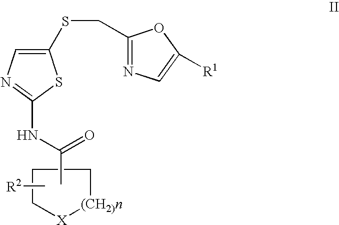
in which
R¹ stands for a C₁-C₁₀-alkyl group,
R² stands for hydrogen or alkyl,
X stands for NR₂ or CHNR₂R₃,
R¹ and R², independently of one another, stand for hydrogen, alkyl, substituted alkyl, cycloalkyl or substituted cycloalkyl, and
n=0, 1, 2, 3, as well as the isomers, enantiomers, diastereomers and salts thereof for the production of a pharmaceutical agent for contraception.

7. Use of the compounds according to claim 5
in which
R¹ stands for a C₁-C₁₀-alkyl group,
R² stands for hydrogen,
X stands for NR₂ or CHNR₂R₃,
R¹ and R², independently of one another, stand for hydrogen, alkyl, substituted alkyl, cycloalkyl or substituted cycloalkyl, and
n=2, as well as the isomers, enantiomers, diastereomers and salts thereof.

8. Use of compounds according to claim 6, comprising general formula IIa, 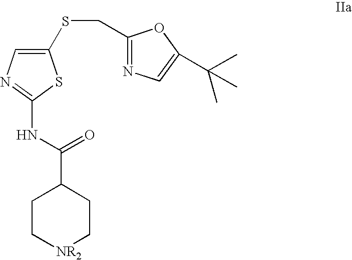
as well as isomers, enantiomers, diastereomers and salts thereof, and in which
R² stands for hydrogen, alkyl, substituted alkyl or cycloalkyl, for the production of a pharmaceutical agent for contraception.

9. Use of the substances according to claim 1 for the production of a pharmaceutical agent for the selective modulation of germ cell maturation.

10. Use of the substances according to claim 8, wherein the germ cells are female germ cells.

11. Use of the substances according to claim 8, whereby the germ cells are male germ cells.

12. Use of the substances according to claim 1 for the production of a pharmaceutical agent for reduction of sperm maturation.

13. Use of the substances according to claim 1 for the production of a pharmaceutical agent for influencing the implantation.

14. Use of the substances according to claim 1 for the production of a pharmaceutical agent for influencing egg cell maturation in vivo.
